(19) United States
(12) Patent Application Publication
(10) Pub. No.: US 2004/0242871 A1
(43) Pub. Date: Dec. 2, 2004

(54) Novel arylheteroalkylamine derivatives

(76) Inventors: Tim Birkinshaw, Leicestershire (GB); Stephen Connolly, Leicestershire (GB); Timothy Luker, Leicestershire (GB); Antonio Mete, Leicestershire (GB); Ian Millichip, Leicestershire (GB)

Correspondence Address: FISH & RICHARDSON PC, 225 FRANKLIN ST, BOSTON, MA, 02110, US

(21) Appl. No.: 10/476,958

(22) PCT Filed: May 6, 2002
(86) PCT No.: PCT/SE02/00876

(30) Foreign Application Priority Data
May 8, 2001 (SE) 0101617-9
Sep. 28, 2001 (SE) 0103271-3

(51) Int. Cl. C07D 253/08; C07D 237/02; C07D 213/72; C07D 241/02

(52) U.S. Cl. 544/183; 544/239; 544/319; 544/406; 546/297

(57) ABSTRACT

There are provided novel compounds of formula (I) wherein R1, R2, R3, R4, R5, R6, T, U, X, Y, V and W are as defined in the specification, and pharmaceutically acceptable salts thereof, and enantiomers and racemates thereof; together with processes for their preparation, compositions containing them and their use in therapy. The compounds are inhibitors of nitric oxide synthase and are thereby particularly useful in the treatment or prophylaxis of inflammatory disease and pain.

### FIELD OF THE INVENTION

The present invention relates to novel arylheteroalkylamine derivatives, processes for their preparation, compositions containing them and their use in therapy.

### BACKGROUND OF THE INVENTION

Nitric oxide is produced in mammalian cells from L-arginine by the action of specific nitric oxide synthases (NOSs). These enzymes fall into two distinct classes—constitutive NOS (cNOS) and inducible NOS (iNOS). At the present time, two constitutive NOSs and one inducible NOS have been identified. Of the constitutive NOSs, an endothelial enzyme (ecNOS) is involved with smooth muscle relaxation and the regulation of blood pressure is and blood flow, whereas the neuronal enzyme (ncNOS) serves as a neurotransmitter and appears to be involved in the regulation of various biological functions such as cerebral ischaemia. Inducible NOS has been particularly implicated in the pathogenesis of inflammatory diseases. Regulation of these enzymes should therefore offer considerable potential in the treatment of a wide variety of disease states (J. E. Macdonald, Ann. Rep. Med. Chem., 1996, 31, 221-230).

Considerable effort has been expended in efforts to identify compounds that act as specific inhibitors of one or more isoforms of the enzyme nitric oxide synthase. The use of such compounds in therapy has also been widely claimed.

### DISCLOSURE OF THE INVENTION

According to the present invention, there is provided a compound of formula (I)

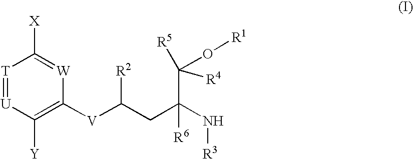

wherein:

X represents H, C1 to 4 alkyl, C1 to 4 alkoxy, halogen, CN, C≡CH, NH₂, NHCH₃, N(CH₃)₂, NO₂, CH₂OH, CHO, COCH₃ or NHCHO; said alkyl or alkoxy group being optionally further substituted by one or more fluorine atoms;

Y represents C1 to 4 alkyl, C1 to 4 alkoxy, halogen, CN, C≡CH, NO₂, CH₂OH, CHO, COCH₃ or NHCHO; said alkyl or alkoxy group being optionally further substituted by one or more fluorine atoms;

T, U and W independently represent CR⁷ or N; and each R⁷ group independently represents H, F or CH₃; and when T represents CR⁷, the group R⁷ may additionally represent OH, Cl, Br, CN, CH₂OH, NO₂, NHR¹³, OR¹⁴ or OSO₂CH₃;

V represents O or S(O)ₙ;

n represents an integer 0, 1 or 2;

R¹ represents H or Me.

R² represents C1 to 4 alkyl, C2 to 4 alkenyl, C2 to 4 alkynyl, C3 to 6 cycloalkyl or a 4 to 8 membered saturated heterocyclic ring incorporating one heteroatom selected from O, S and N; any of said groups being optionally further substituted by C1 to 4 alkyl, C1 to 4 alkoxy, C1 to 4 alkylthio, C3 to 6 cycloalkyl, halogen or phenyl; said phenyl group being optionally further substituted by one or more substituents selected independently from halogen, C1 to 4 alkyl, C1 to 4 alkoxy, CF₃, OCF₃, CN or NO₂;

or R represents phenyl or a five or six membered aromatic heterocyclic ring containing 1 to 3 heteroatoms independently selected from O, S and N; said phenyl or aromatic heterocyclic ring being optionally substituted by one or more substituents selected independently from halogen, C1 to 4 alkyl, C1 to 4 alkoxy, OH, CN, NO₂ or NR⁹R¹⁰; said alkyl or alkoxy group being optionally further substituted by one or more fluorine atoms;

R³ represents H, C1 to 4 alkyl or C3 to 6 cycloalkyl; said alkyl group being optionally substituted by C1 to 4 alkoxy, halogen, hydroxy, NR¹¹R¹², phenyl or a five or six membered aromatic or saturated heterocyclic ring containing 1 to 3 heteroatoms independently selected from O, S and N; said phenyl or aromatic heterocyclic ring being optionally further substituted by halogen, C1 to 4 alkyl, C1 to 4 alkoxy, CF₃, OCF₃, CN or NO₂.

R⁴, R⁵, R⁶, R⁹, R¹⁰, R¹¹, R¹², R¹³ and R¹⁴ independently represent H or C1 to 4 alkyl;

or a pharmaceutically acceptable salt, enantiomer or racemate thereof.

It will be recognised that compounds of formula (I) wherein U represents N and T represents CR⁷ and R⁷ represents OH may exist in the alternative tautomeric form (Ia).

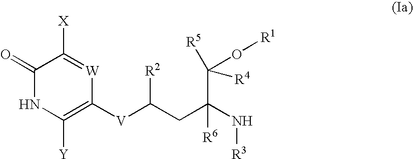

It is to be understood that all such possible tautomeric forms and mixtures thereof are included within the scope of the invention.

In one embodiment, X and Y independently represent C1 to 4 alkyl, C1 to 4 alkoxy, halogen, CN, C≡CH, NO₂, CHO, COCH₃ or NHCHO; said alkyl or alkoxy group being optionally further substituted by one or more fluorine atoms; and T, U and W independently represent CR⁷ or N; and each R⁷ group independently represents H, F or CH₃.

The compounds of formula (I) and their pharmaceutically acceptable salts, enantiomers and racemates have the advantage that they are inhibitors of the enzyme nitric oxide synthase (NOS). In particular, the compounds of formula (I) and their pharmaceutically acceptable salts, enantiomers and racemates have the advantage that they are inhibitors of the inducible isoform of the enzyme nitric oxide synthase (iNOS).

The invention further provides a process for the preparation of compounds of formula (I) or a pharmaceutically acceptable salt, enantiomer or racemate thereof.

According to the invention there is also provided a compound of formula (I), or a pharmaceutically acceptable salt, enantiomer or racemate thereof, for use as a medicament.

Another aspect of the invention provides the use of a compound of formula (I) or a pharmaceutically acceptable salt, enantiomer or racemate thereof, in the manufacture of a medicament, for the treatment or prophylaxis of diseases or conditions in which inhibition of nitric oxide synthase activity is beneficial.

A more particular aspect of the invention provides the use of a compound of formula (I) or a pharmaceutically acceptable salt, enantiomer or racemate thereof, in the manufacture of a medicament, for the treatment or prophylaxis of inflammatory disease.

According to the invention, there is also provided a method of treating, or reducing the risk of, diseases or conditions in which inhibition of nitric oxide synthase activity is beneficial which comprises administering to a person suffering from or at risk of, said disease or condition, a therapeutically effective amount of a compound of formula (I) or a pharmaceutically acceptable salt, enantiomer or racemate thereof.

More particularly, there is also provided a method of treating, or reducing the risk of, inflammatory disease in a person suffering from or at risk of, said disease, wherein the method comprises administering to the person a therapeutically effective amount of a compound of formula (I) or a pharmaceutically acceptable salt, enantiomer or racemate thereof.

The compounds of the present invention may also be used advantageously in combination with a second pharmaceutically active substance; particularly in combination with a cyclooxygenase inhibitor; more particularly in combination with a selective inhibitor of the inducible isoform of cyclooxygenase (COX-2). Thus, in a further aspect of the invention there is provided the use of a compound of formula (I) or a pharmaceutically acceptable salt, enantiomer or racemate thereof, in combination with a COX-2 inhibitor for the treatment of inflammation, inflammatory disease and inflammatory related disorders. And there is also provided a method of treating, or reducing the risk of, inflammation, inflammatory disease and inflammatory related disorders in a person suffering from or at risk of, said disease or condition, wherein the method comprises administering to the person a therapeutically effective amount of a compound of formula (I) or a pharmaceutically acceptable salt, enantiomer or racemate thereof in combination with a COX-2 inhibitor.

In one embodiment, V represents S(O)ₙ and n represents 0.

In another embodiment, V represents O.

In another embodiment, X and Y independently represent Br, Cl, CH₃, CH₂F, CHF₂, CF₃, OCH₃ or CN. In yet another embodiment Y represents CN.

In one embodiment, R¹ represents H.

In another embodiment, R² represents phenyl or a five or six membered aromatic heterocyclic ring containing 1 to 3 heteroatoms independently selected from O, S and N. In a further embodiment, R² represents phenyl, pyridyl, isoxazolyl, isothiazolyl or thiazolyl.

In a yet further embodiment, R² represents phenyl.

In one embodiment, R³ represents H.

In another embodiment R⁴, R⁵ and R⁶ each represent H.

In another embodiment, T, U and W independently represent N, CH or CF. In another embodiment U represents N or CH. In yet another embodiment W represents N or CH.

In one embodiment, each of T, U and W represents CR⁷.

In one embodiment, one of T, U and W represents N and the other two represent CR⁷.

In a particular embodiment, the compounds of formula (I) have the (1R, 3S) absolute stereochemistry.

In one particular aspect the invention relates to compounds of formula (I) wherein V represents O or S; X and Y independently represent Br, Cl, CH₃, CH₂F, CHF₂, CF₃, OCH₃ or CN; R¹, R³, R⁴, R⁵ and R⁶ each represent H; R² represents phenyl, pyridyl, isoxazolyl, isothiazolyl or thiazolyl; T represents N, CH or CF; U represents N or CH; W represents N or CH; and the compounds have the (1R, 3S) absolute configuration; and pharmaceutically acceptable salts thereof.

In another particular aspect the invention relates to compounds of formula (I) wherein V represents O or S; X and Y independently represent Br, Cl, CH₃, CH₂F, CHF₂, CF₃, OCH₃ or CN; R¹, R³, R⁴, R⁵ and R⁶ each represent H; R² represents phenyl, pyridyl, isoxazolyl, isothiazolyl or thiazolyl; one of T, U and W represents N and the other two represent CR⁷; and the compounds have the (1R, 3S) absolute configuration; and pharmaceutically acceptable salts thereof.

Particular compounds of the invention include:

2-[[(1R,3S)-3-amino-4-hydroxy-1-phenylbutyl]thio]-6-methyl-3-pyridinecarbonitrile;

2-[[(3S)-3-amino-4-hydroxy-1-(3-isoxazolyl)butyl]thio]-6-methyl-3-pyridinecarbonitrile;

4-[[(1R,3S)-3-amino-4-hydroxy-1-phenylbutyl]thio]-6-methyl-3-pyridinecarbonitrile;

3-[[(1R,3S)-3-amino-4-hydroxy-1-phenylbutyl]thio]-5-(trifluoromethyl)-2-pyridinecarbonitrile;

2-[[(1R,3S)-3-amino-4-hydroxy-1-phenylbutyl]thio]-6-(difluoromethyl)-3-pyridinecarbonitrile;

2-[[(1R,3S)-3-amino-4-hydroxy-1-phenylbutyl]thio]-6-(fluoromethyl)-3-pyridinecarbonitrile;

2-[[(1R,3S)-3-amino-4-hydroxy-1-(3-pyridinyl)butyl]oxy]-4-chloro-5-fluorobenzonitrile;

2-[[(1R,3S)-3-amino-4-hydroxy-1-(2-thiazolyl)butyl]oxy]-4-chloro-5-fluorobenzonitrile;

2-[[(1R,3S)-3-amino-4-hydroxy-1-(5-isothiazolyl)butyl]oxy]-4-chloro-5-fluorobenzonitrile;

4-[[(1R,3S)-3-amino-4-hydroxy-1-phenylbutyl]thio]-6-methoxy-3-pyridinecarbonitrile;

4-[[(1R,3R)-3-amino-4-hydroxy-1-phenylbutyl]thio]-6-methoxy-3-pyridinecarbonitrile;

4-[[(1S,3R)-3-amino-4-hydroxy-1-phenylbutyl]thio]-6-methoxy-3-pyridinecarbonitrile;

4-[[(1S,3S)-3-amino-4-hydroxy-1-phenylbutyl]thio]-6-methoxy-3-pyridinecarbonitrile;

4-[[(1R,3S)-3-amino-4-hydroxy-1-phenylbutyl]thio]-6-(difluoromethoxy)-3-pyridinecarbonitrile;

2-[[(1R,3R)-3-amino-4-hydroxy-1-phenylbutyl]thio]-6-methyl-3-pyridinecarbonitrile;

4-[[(1R,3S)-3-amino-4-hydroxy-1-phenylbutyl]thio]-6-(²H₃)methoxy-3-pyridinecarbonitrile;

2-[[(1R,3S)-3-amino-4-hydroxy-1-phenylbutyl]thio]-6-ethyl-3-pyridinecarbonitrile;

2-[[(1R,3S)-3-amino-4-hydroxy-1-phenylbutyl]thio]-6-(1-methylethyl)-3-pyridinecarbonitrile;

2-[[(1R,3S)-3-amino-4-hydroxy-1-phenylbutyl]thio]-6-methyl-3-pyridinemethanol;

6-acetyl-2-[[(1R,3S)-3-amino-4-hydroxy-1-phenylbutyl]thio]-3-pyridinecarbonitrile;

2-[[(1R,3S)-3-amino-4-hydroxy-1-phenylbutyl]thio]-6-(hydroxymethyl)-3-pyridinecarbonitrile;

2-[[(1R,3S)-3-amino-4-hydroxy-1-phenylbutyl]thio]-3-pyridinecarbonitrile;

(β¹S,δ¹R)-β-amino-δ-[(2,5-dichloro-4-pyridinyl)thiobenzenebutanol];

2-[[(1R,3S)-3-amino-4-hydroxy-1-phenylbutyl]thio]-5-fluoro-6-methoxy-3-pyridinecarbonitrile;

4-[[(1R,3S)-3-amino-4-hydroxy-1-phenylbutyl]thio]-6-(dimethylamino)-3-pyridinecarbonitrile;

4-[[(1R,3S)-3-amino-4-hydroxy-1-phenylbutyl]thio]-6-(methylamino)-3-pyridinecarbonitrile;

(β¹S,δ¹R)-β-amino-δ-[(5-bromo-2-methoxy-4-pyridinyl)thio]-benzenebutanol;

(β¹S,δ¹R)-β-amino-δ-[(5-chloro-2-methoxy-4-pyridinyl)thio]-benzenebutanol;

4-[[(1R,3S)-3-amino-4-hydroxy-1-phenylbutyl]thio]-6-ethoxy-3-pyridinecarbonitrile;

3-[[(1R,3S)-3-amino-4-hydroxy-1-phenylbutyl]thio]-5-(trifluoromethyl)-2-pyridinecarbonitrile;

3-[[(1R,3S)-3-amino-4-hydroxy-1-phenylbutyl]thio]-1,6-dihydro-5-methyl-6-oxo-2-pyridinecarbonitrile;

3-[[(1R,3S)-3-amino-4-hydroxy-1-phenylbutyl]thio]-5-chloro-2-pyridinecarbonitrile;

6-amino-4-[[(1R,3S)-3-amino-4-hydroxy-1-phenylbutyl]thio]-3-pyridinecarbonitrile;

3-[[(1R,3S)-3-amino-4-hydroxy-1-phenylbutyl]thio]-5-methyl-2-pyridinecarbonitrile;

4-[[(1R,3S)-3-amino-1-(2-fluorophenyl)-4-hydroxybutyl]thio]-6-methoxy-3-pyridinecarbonitrile;

2-[[(1R,3S)-3-amino-1-(4-fluorophenyl)-4-hydroxybutyl]oxy]-6-trifluoromethyl-3-pyridinecarbonitrile;

2(2S)-amino-4(4R)-(3-fluorophenyl)-4-[(4-methoxy-2-nitrophenyl)thio]butan-1-ol;

2(2S)-amino-4(4R)-(3-fluorophenyl)-4-[(4-chloro-2-nitrophenyl)thio]butan-1-ol;

2(2S)-amino-4(4R)-(3-fluorophenyl)-4-[(5-amino-4-chloro-2-nitrophenyl)thio]butan-1-ol;

2(2S)-amino-4(4R)-(3-fluorophenyl)-4-[(4-hydroxymethyl)-2-nitrophenyl)thio]butan-1-ol;

2(2S)-amino-4(4R)-(3-fluorophenyl)-4-[(4-fluoro-2-nitrophenyl)thio]butan-1-ol;

2(2S)-amino-4(4R)-(3-fluorophenyl)-4-[(3,5-dichloro-2-pyridyl)thio]butan-1-ol;

4-[[(1R,3S)-3-amino-4-hydroxy-1-phenylbutyl]thio]-3-chlorobenzonitrile;

4-chloro-2-[[(1R,3S)-3-(ethylamino)-4-hydroxy-1-(2-thiazolyl)butyl]oxy]-5-fluorobenzonitrile;

2-[[(1R,3S)-3-amino-4-hydroxy-1-(5-thiazolyl)butyl]oxy]-5-fluoro-benzonitrile;

2-[[(1R,3S)-3-amino-4-methoxy-1-phenylbutyl]thio]-6-methyl-3-pyridinecarbonitrile;

2-[[(1R,3S)-3-amino-4-hydroxy-4-methyl-1-phenylpentyl]oxy]4-chloro-5-fluorobenzonitrile;

2-[[(1S,3S)-3-amino-4-hydroxy-1-propylbutyl]oxy]-4-chloro-5-fluorobenzonitrile;

2-[[(1S)-1-[(2S)-2-amino-3-hydroxypropyl]pentyl]thio]-6-methyl-3-pyridinecarbonitrile;

2-[[(1S,3S)-3-amino-4-hydroxy-1-(2-methylpropyl)butyl]thio]-6-methyl-3-pyridinecarbonitrile;

2-[[(3S)-3-amino-4-hydroxy-1-(5-isoxazolyl)butyl]thio]-6-methyl-3-pyridinecarbonitrile;

2-[[(3S)-3-amino-4-hydroxy-1-(5-isoxazolyl)butyl]oxy]-6-(trifluoromethyl)-3-pyridinecarbonitrile;

2-[[3-(3S)-amino-4-hydroxy-1-(1R)-(2-thienyl)butyl]oxy]-4-chloro-5-fluorobenzonitrile;

2-[[3-(3S)-amino-4-hydroxy-1(1R)-(3-thienyl)butyl]oxy]-4-chloro-5-fluorobenzonitrile;

2-[[(1R,3S)-3-amino-4-hydroxy-1-(3-pyridinyl)butyl]thio]-4-(trifluoromethyl)benzonitrile;

2-[[(1R,3S)-3-amino-4-hydroxy-1-(5-pyrimidyl)butyl]thio]-4-chlorobenzonitrile;

2-[[(1R,3S)-3-amino-4-hydroxy-1-(3-pyridinyl)butyl]thio]-4-chloro-5-fluorobenzonitrile;

2-[[(1R,3S)-3-amino-4-hydroxy-1-(3-pyridyl)butyl]thio]-4-bromobenzonitrile;

2-[[(1R,3S)-3-amino-4-hydroxy-1-(2-thiazolyl)butyl]oxy]-5-fluoro-6-methyl-3-pyridinecarbonitrile;

4-[[(1R,3S)-3-amino-1-(3-fluoro-2-thienyl)-4-hydroxybutyl]thio]-6-methoxy-3-pyridinecarbonitrile;

2-[[(1R,3S)-3-amino-1-(4-chloro-5-thiazolyl)-4-hydroxybutyl]oxy]-4-chloro-5-fluorobenzonitrile;

2-[[(1R,3S)-3-amino-4-hydroxy-1-phenylbutyl]thio]-5-nitrobenzonitrile;

2-[[(1R,3S)-3-amino-4-hydroxy-1-phenylbutyl]thio]-5-chloro-3-pyridinecarbonitrile;

β-amino-δ-[(4-amino-2-nitrophenyl)thio]-(β¹S,δ¹R)-benzenebutanol;

2-[[(1R,3S)-3-amino-4-hydroxy-1-phenylbutyl]thio]-5-bromo-benzonitrile;

and pharmaceutically acceptable salts, enantiomers or racemates thereof.

Unless otherwise indicated, the term “C1 to 4 alkyl” referred to herein denotes a straight or branched chain alkyl group having from 1 to 4 carbon atoms. Examples of such groups include methyl, ethyl, n-propyl, i-propyl, n-butyl, i-butyl and t-butyl.

Unless otherwise indicated, the term “C3 to 6 cycloalkyl” referred to herein denotes a cycloalkyl group having from 3 to 6 carbon atoms. Examples of such groups include cyclopropyl, cyclopentyl and cyclohexyl.

Unless otherwise indicated, the term “C2 to 4 alkenyl” referred to herein denotes a straight or branched chain alkyl group having from 2 to 4 carbon atoms incorporating at least one carbon-carbon double bond. Examples of such groups include ethenyl, propenyl and butenyl.

Unless otherwise indicated, the term “C2 to 4 alkynyl” referred to herein denotes a straight or branched chain alkyl group having from 2 to 4 carbon atoms incorporating at least one carbon-carbon triple bond. Examples of such groups include ethynyl, propynyl, and butynyl.

Unless otherwise indicated, the term “C1 to 4 alkoxy” referred to herein denotes a straight or branched chain alkoxy group having from 1 to 4 carbon atoms. Examples of such groups include methoxy, ethoxy, n-propoxy, i-propoxy and t-butoxy.

The term “C1 to 4 alkylthio” is to be interpreted analogously.

Unless otherwise indicated, the term “halogen” referred to herein denotes fluoro, chloro, bromo and iodo.

Examples of a 4 to 8 membered saturated azacyclic ring optionally incorporating one further heteroatom selected from O, S or N include pyrrolidine, piperidine, piperazine, morpholine and perhydroazepine.

Examples of a 4 to 8 membered saturated heterocyclic ring incorporating one heteroatom selected from O, S or N include pyrrolidine, piperidine, tetrahydrofuran and perhydroazepine.

Examples of a five or six membered aromatic heterocyclic ring containing 1 to 3 heteroatoms independently selected from O, S and N include furan, thiophene, pyridine, thiazole, imidazole, oxazole, triazole, oxadiazole, thiadiazole and pyrimidine.

Examples of a five or six membered saturated heterocyclic ring containing 1 to 3 heteroatoms independently selected from O, S and N include pyrrolidine, tetrahydrofuran, piperidine and piperazine.

Examples of a “C1 to 4 alkyl or C1 to 4 alkoxy optionally further substituted by one or more fluorine atoms” include CH₂F, CHF₂, CF₃, CF₃CF₂, CF₃CH₂, CH₂FCH₂, CH₃CF₂, CF₃CH₂CH₂, OCF₃ and OCH₂CF₃.

According to the invention, we further provide a process for the preparation of compounds of formula (I), or a pharmaceutically acceptable salt, enantiomer or racemate thereof which comprises:

(a) reaction of a compound of formula (II)

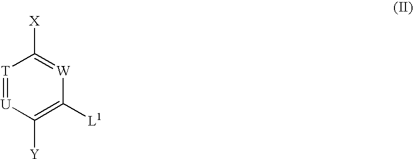

wherein T, U, X, Y and W are as defined in formula (I) and L¹ represents a leaving group,

with a compound of formula (III)

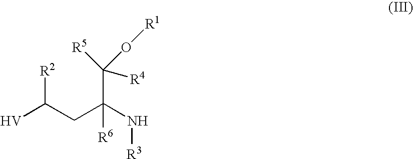

wherein R¹, R², R³, R⁴, R⁵, R⁶ and V are as defined in formula (I); or

(b) reaction of a compound of formula (IV)

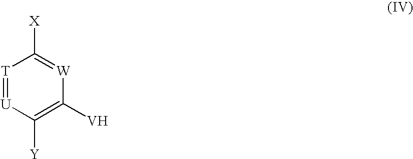

wherein T, U, W, X, Y and V are as defined in formula (I),

with a compound of formula (V)

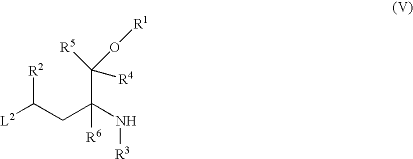

wherein R¹, R², R³, R⁴, R⁵ and R⁶ are as defined in formula (I) and L² is a leaving group;

and where desired or necessary converting the resultant compound of formula (I), or another salt thereof, into a pharmaceutically acceptable salt thereof; or converting one compound of formula (I) into another compound of formula (I); and where desired converting the resultant compound of formula (I) into an optical isomer thereof.

In process (a), the reaction is performed by treating a nucleophile of formula (III) with an electrophile of formula (II) in an inert solvent. Suitable leaving groups L¹ include sulphonates and halides, particularly fluoride or chloride. The reaction is generally performed in the presence of a non-nucleophilic base such as sodium hydride or caesium carbonate. Suitable organic solvents are those such as N,N-dimethylformamide, N-methyl-2-pyrrolidinone, tetrahydrofuran, acetonitrile and dimethylsulfoxide. The reaction is generally conducted at a temperature between 0° C. and the boiling point of the solvent.

In process (b), the reactants (IV) and (V) are coupled together in a suitable inert solvent such as tetrahydrofuran using, for example, Mitsunobu conditions. Thus, for example, the reactants are treated with a phosphine derivative and an azo derivative at a suitable temperature, generally between 0° C. and the boiling point of the solvent. Suitable phosphine derivatives include triphenylphosphine and tributylphosphine. Suitable azo derivatives include diethyl azodicarboxylate, diisopropyl azodicarboxylate and 1,1′-(azodicarbonyl)dipiperidine. Suitable leaving groups L² include hydroxy.

Alternatively in process (b), the reaction is performed by treating a nucleophile of formula (IV) with an electrophile of formula (V) in an inert solvent. Suitable leaving groups L² include sulphonates and halides, particularly chloride or bromide. The reaction is generally performed in the presence of a non-nucleophilic base such as sodium hydride or caesium carbonate. Suitable organic solvents are those such as N,N-dimethylformamide, N-methyl-2-pyrrolidinone, tetrahydrofuran and dimethylsulfoxide. The reaction is generally conducted at a temperature between 0° C. and the boiling point of the solvent.

It will be apparent to a person skilled in the art that in the above processes it may be desirable or necessary to protect an amine or hydroxyl or other potentially reactive group. Suitable protecting groups and details of processes for adding and removing such groups may be found by reference to the standard text “Protective Groups in Organic Synthesis”, 3rd Edition (1999) by Greene and Wuts.

In one preferred embodiment, amine groups are protected as carbamate derivatives, for example, as t-butyloxycarbamates.

In another particularly preferred embodiment, the amine and hydroxyl groups of compounds wherein R¹ represents hydrogen are protected simultaneously as a cyclic carbamate, such as in formula (VI), or as a cyclic hemi-aminal as in formula (VII).

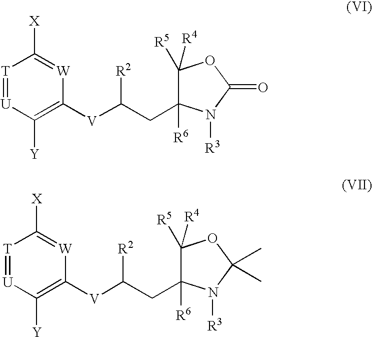

Specific examples of the use of protecting groups are given in the Examples section.

The present invention includes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tartaric, lactic, pyruvic, acetic, succinic, fumaric, maleic, methanesulphonic and benzenesulphonic acids.

Salts of compounds of formula (I) may be formed by reacting the free base, or a salt, enantiomer or racemate thereof, with one or more equivalents of the appropriate acid. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin.

Certain novel intermediates of formulae (III), (V), (VI) and (VII) form another aspect of the invention.

Compounds of formula (III) may be prepared by reaction of a compound of formula (VIII)

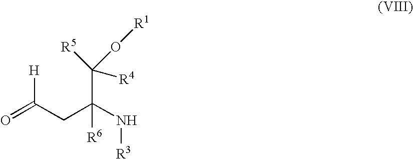

wherein R¹, R², R³, R⁴, R⁵ and R⁶ are as defined in formula (I),

with an organometallic derivative, R²-M, wherein R² is as defined in formula (I) and M represents a metallic residue such as lithium or magnesium-halide. The resulting compound of formula (III) wherein V represents oxygen may then be subsequently converted into compounds of formula (III) wherein V represents sulphur.

Alternatively, compounds of formula (III) may be prepared by reaction of an amide of formula (IX)

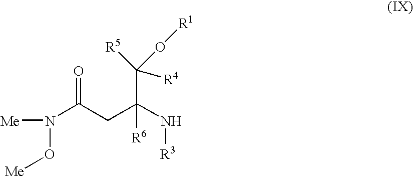

wherein R¹, R³, R⁴, R⁵ and R⁶ are as defined in formula (I),

with an organometallic derivative, R²-M, wherein R² is as defined in formula (I) and M represents a metallic residue such as lithium or magnesium-halide, followed by reduction of the resulting ketone to the corresponding alcohol (III).

Compounds of formulae (II), (IV), (VIII) and (IX) are either known or may be prepared by conventional methods that will be readily apparent to the man skilled in the art.

Intermediate compounds may be used as such or in protected form. Protecting groups and details of processes for their removal may be found by reference to the standard text “Protective Groups in Organic Synthesis”, 3rd Edition (1999) by Greene and Wuts.

The compounds of the invention and intermediates thereto may be isolated from their reaction mixtures and, if necessary further purified, by using standard techniques.

The compounds of formula I may exist in enantiomeric forms. Therefore, all enantiomers, diastereomers, racemates and mixtures thereof are included within the scope of the invention. The various optical isomers may be isolated by separation of a racemic mixture of the compounds using conventional techniques, for example, fractional crystallisation, or HPLC.

Intermediate compounds may also exist in enantiomeric forms and may be used as purified enantiomers, diastereomers, racemates or mixtures.

The compounds of formula (I), and their pharmaceutically acceptable salts, enantiomers and racemates, are useful because they possess pharmacological activity in animals. In particular, the compounds are active as inhibitors of the enzyme nitric oxide synthase. More particularly, they are inhibitors of the inducible isoform of the enzyme nitric oxide synthase and as such are predicted to be useful in therapy, for example, as anti-inflammatory agents. They may also have utility as inhibitors of the neuronal isoform of the enzyme nitric oxide synthase.

The compounds and their pharmaceutically acceptable salts, enantiomers and racemates are indicated for use in the treatment or prophylaxis of diseases or conditions in which synthesis or oversynthesis of nitric oxide synthase forms a contributory part. In particular, the compounds are indicated for use in the treatment of inflammatory conditions in mammals including man.

Conditions that may be specifically mentioned are:

osteoarthritis, rheumatoid arthritis, rheumatoid spondylitis, gouty arthritis and other arthritic conditions, inflamed joints;

eczema, psoriasis, dermatitis or other inflammatory skin conditions such as sunburn; inflammatory eye conditions including uveitis, glaucoma and conjunctivitis; lung disorders in which inflammation is involved, for example, asthma, bronchitis, chronic obstructive pulmonary disease, pigeon fancier's disease, farmer's lung, acute respiratory distress syndrome;

bacteraemia, endotoxaemia (septic shock), aphthous ulcers, gingivitis, pyresis, pain, meningitis and pancreatitis;

conditions of the gastrointestinal tract including inflammatory bowel disease, Crohn's disease, atrophic gastritis, gastritis varialoforme, ulcerative colitis, coeliac disease, regional ileitis, peptic ulceration, irritable bowel syndrome, reflux oesophagitis, damage to the gastrointestinal tract resulting from infections by, for example, Helicobacter pylori, or from treatments with non-steroidal anti-inflammatory drugs;

and other conditions associated with inflammation.

The compounds will also be useful in the treatment and alleviation of acute pain or persistent inflammatory pain or neuropathic pain or pain of a central origin.

We are particularly interested in the conditions inflammatory bowel disease, rheumatoid arthritis, osteoarritis, chronic obstructive pulmonary disease and pain.

The compounds of formula (I) and their pharmaceutically acceptable salts, enantiomers and racemates may also be useful in the treatment or prophylaxis of diseases or conditions in addition to those mentioned above. For example, the compounds may be useful in the treatment of atherosclerosis, cystic fibrosis, hypotension associated with septic and/or toxic shock, in the treatment of dysfunction of the immune system, as an adjuvant to short-term immunosuppression in organ transplant therapy, in the control of onset of diabetes, in the maintenance of pancreatic function in diabetes, in the treatment of vascular complications associated with diabetes and in co-therapy with cytokines, for example TNF or interleukins.

The compounds of formula (I) may also be useful in the treatment of hypoxia, for example in cases of cardiac arrest and stroke, neurodegenerative disorders including nerve degeneration and/or nerve necrosis in disorders such as ischaemia, hypoxia, hypoglycaemia, epilepsy, and in external wounds (such as spinal cord and head injury), hyperbaric oxygen convulsions and toxicity, dementia, for example pre-senile dementia, Alzheimer's disease and AIDS-related dementia, Sydenham's chorea, Parkinson's disease, Tourette's syndrome, Huntington's disease, amyotrophic lateral sclerosis, multiple sclerosis, muscular dystrophy, Korsakoff's disease, imbecility relating to a cerebral vessel disorder, sleeping disorders, schizophrenia, depression, pain, autism, seasonal affective disorder, jet-lag, depression or other symptoms associated with premenstrual syndrome (PMS), anxiety and septic shock. Compounds of formula (I) may also be expected to show activity in the prevention and reversal of drug addiction or tolerance such as tolerance to opiates and diazepines, treatment of drug addiction, treatment of migraine and other vascular headaches, neurogenic inflammation, in the treatment of gastrointestinal motility disorders, cancer and in the induction of labour.

We are particularly interested in the conditions stroke, Alzheimer's disease, Parkinson's disease, multiple sclerosis, schizophrenia, migraine, cancer, septic shock and pain.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dosage administered will, of course, vary with the compound employed, the mode of administration and the treatment desired. However, in general, satisfactory results are obtained when the compounds are administered at a dosage of the solid form of between 1 mg and 2000 mg per day.

The compounds of formula (I), and pharmaceutically acceptable derivatives thereof, may be used on their own, or in the form of appropriate pharmaceutical compositions in which the compound or derivative is in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988. The pharmaceutical composition preferably comprises less than 80% and more preferably less than 50% of a compound of formula (I), or a pharmaceutically acceptable salt, enantiomer or racemate thereof.

According to the invention, we further provide a pharmaceutical composition comprising a compound of formula (I), or a pharmaceutically acceptable salt, enantiomer or racemate thereof, in admixture with a pharmaceutically acceptable adjuvant, diluent or carrier.

There is also provided a process for the preparation of such a pharmaceutical composition which comprises mixing the ingredients.

The compounds of formula (I), and pharmaceutically acceptable derivatives thereof, may also be advantageously used in combination with a COX inhibitor, more particularly in combination with a COX-2 inhibitor. Particularly preferred COX-2 inhibitors are Celecoxib and MK-966. The NOS inhibitor and the COX-2 inhibitor may either be formulated together within the same pharmaceutical composition for administration in a single dosage unit, or each component may be individually formulated such that separate dosages may be administered either simultaneously or sequentially.

The invention is illustrated, but in no way limited, by the following examples:

The following abbreviations are used:- DMSO (dimethylsulfoxide), DMF (N,N-dimethylformamide), THF (tetrahydrofuran), NMP (N-methylpyrrolidinone).

### EXAMPLE 1

### 2-[[(1R,3S)-3-Amino-4-hydroxy-1-phenylbutyl]thio]-6-methyl-3-pyridinecarbonitrile ethanedioate

a) 1,1-Dimethylethyl (4S)-4-[(2S)-2-hydroxy-2-phenylethyl]-2,2-dimethyl-3-oxazolidinecarboxylate and 1,1-Dimethylethyl (4S)-4-[(2R)-2-hydroxy-2-phenylethyl]-2,2-dimethyl-3-oxazolidinecarboxylate

To a stirred solution of 1,1-dimethylethyl (4S)-2,2-dimethyl-4-(2-oxoethyl)-3-oxazolidinecarboxylate (6.9 g) in dry THF (100 ml) at 0° C. and under nitrogen, was added phenyl magnesium bromide (34 ml, 1M in THF). During the addition an exotherm to 20° C. occurred, and the mixture was kept at this temperature for 3 h. The reaction mixture was quenched with 5% aqueous citric acid (100 ml) and the products extracted into ethyl acetate (150 ml). The organic extract was dried (MgSO₄) and concentrated to an oil. The crude mixture of diastereomers was purified by chromatography (silica, 10% diethyl ether/isohexane as eluent) to give the (4S, 2S) sub-title compound (3.5 g, 38%) as a colourless solid.

¹H NMR 400 MHz (CDCl₃) 7.4-7.2 (5H, m), 4.88 (1H, d), 4.65 (1H, m), 4.35 (1H, m), 4.0 (1H, m), 3.65 (1H, d), 2.1-2 (1H, m), 1.85-1.95 (1H, m), 1.6 (3H, s), 1.49 (12H, s).

Further elution gave the (4S, 2R) sub-title compound (2.5 g, 27%) as a colourless solid.

¹H NMR 400 MHz (CDCl₃) 7.4-7.3 (5H, brs), 4.77-4.73 (1H, m), 4.3-3.7 (3H, m), 2.2-2 (2H, m), 1.6-1.4 (15H, m).

b) 1,1-Dimethylethyl (4S) 4-[(2R)-2-(benzoylthio)-2-phenylethyl]-2,2-dimethyl-3-oxazolidinecarboxylate

To a solution of the (4S, 2S) product from step (a) (3 g) and tris(4-chlorophenyl)phosphine in dry THF at 0° C. was added diisopropylazodicarboxylate (1.84 ml) dropwise over 5 minutes. After the addition was complete the mixture was stirred for 20 minutes and then thiobenzoic acid (1.1 ml) added. The cooling bath was removed and stirring continued overnight. The mixture was concentrated and the residue was purified by chromatography (silica, 10% diethyl ether/isohexane as eluent) to give the sub-title compound (1.2 g, 29%) as a yellow coloured solid.

MS APCI +ve ^{m}/z 342 ([M+H-Boc]⁺).

c) 1,1-Dimethylethyl (4S) 4-[(2R)-2-[(3-cyano-6-methyl-2-pyridinyl)thio-]-2-phenyletyl]-2,2-dimethyl-3-oxazolidinecarboxylate

A mixture of the product from step (b) (440 mg), 2-chloro-6-methyl-3-pyridinecarbonitrile (229 mg), sodium carbonate (159 mg) and water (1 ml) in methanol (10 ml) was stirred at room temperature for 17 h. The mixture was diluted with water (50 ml) and extracted with diethyl ether (2×50 ml). The combined extracts were dried (MgSO₄) and concentrated to an oil and purified by chromatography (silica, 10% diethyl ether/isohexane as eluent) to afford the protected amino alcohol.

MS APCI+ve ^{m}/z 454 [M+H]⁺.

d) 2-[[(1R,3S)-3-Amino-4-hydroxy-1-phenylbutyl]thio]-6-methyl-3-pyridinecarbonitrile ethanedioate

The total product from step (c) was dissolved in ethylene glycol (2 ml), a crystal of pyridinium tosylate added and the solution heated at 190° C. for 10 minutes. The mixture was cooled to ambient temperature, diluted with methanol (50 ml), and the solution stirred with SCX resin. The resin was collected by filtration and treated with methanolic ammonia. The ammonia solution was concentrated to dryness and the residue purified by chromatography (silica, 10% 7M methanolic ammonia in dichloromethane as eluent) to afford the free base (70 mg, 22%). The amine was converted into the ethanedioate salt using one equivalent of oxalic acid in ethanol to afford the title compound.

MS APCI+ve ^{m}/z 314 [M+H]⁺.

¹H NMR 400 MHz (d₆-DMSO) 8.1-7.2 (7H, m), 5.33 (1H, t), 3.6-3.4 (2H, m), 2.93 (1H, br m), 2.59 (3H, s), 2.35-2.2 (2H, m).

### EXAMPLE 2

### 2-[[(3S)-3-Amino-4-hydroxy-1-(3-isoxazolyl)butyl]thio]-6-methyl-3-pyridinecarbonitrile ethanedioate

a) (4S)-4-[2-(3-Isoxazolyl)-2-oxoethyl]-2-oxazolidinone

Dibromoethane (0.4 g) was added to a suspension of zinc dust (1.3 g) in dry THF (4 ml) under nitrogen, and the mixture heated to ca. 60° C. After immediately cooling to room temperature, further dibromoethane (0.4 g) was added and the heat/cool cycle was repeated. THF (5 ml) and chlorotrimethylsilane (0.2 ml) were added and the mixture stirred for 2 minutes. A solution of (4R)-4-(iodomethyl)-2-oxazolidinone (2.26 g) in THF (4 ml) was added dropwise (a slight exotherm was observed) and the reaction heated at 30° C. for 1 h. After cooling to room temperature, THF (6 ml) was added and the suspension was left to stand for 1 h. The supernatant was transferred by cannula over 5 minutes into a solution of lithium chloride (0.84 g) and copper (I) cyanide (0.88 g) in THF (8 ml) at −78° C. under nitrogen (the salts had previously been stirred together for 10 minutes at room temperature). The mixture was warmed to 0° C., re-cooled to −78° C. and a solution of 3-isoxazolecarbonyl chloride (0.78 g) in THF (1 ml) added. After 1 h, the mixture was warmed to −10° C. and left to slowly warm to room temperature over 16 h. The reaction mixture was poured into a mixture of ethyl acetate and saturated ammonium chloride solution and the mixture filtered through celite. The organic layer was then separated and washed with water and brine and dried (MgSO₄). The solvent was evaporated and the residue purified by chromatography (silica, 25 to 100% ethyl acetate/isohexane as eluent) to give the sub-title compound (0.82 g, 70%) as an oily solid.

¹H NMR 400 MHz (d₆-DMSO) 9.15 (1H, s), 7.71 (1H, s), 6.95 (1H, s), 4.50 (1H, t), 4.26 (1H, quintet), 4.03 (1H, dd), 3.44 (1H, dd), 3.30 (1H, m).

b) (4S)-4-[2-Hydroxy-2-(3-isoxazolyl)ethyl]-2-oxazolidinone

Borane (4.16 ml of a 1M solution in THF) was added to a solution of (R)-2-methyl-CBS-oxazaborolidine (0.42 ml, 1M solution in toluene) in THF (4 ml) at 0° C. After 10 minutes, is a solution of (4S)-4-[2-(3-isoxazolyl)-2-oxoethyl]-2-oxazolidinone (0.82 g) in THF (3 ml) was added over 5 minutes and the resultant solution was stirred at 0° C. for 1 h and at 20° C. for 18 h. Methanol (25 ml) was added and the mixture was stirred for 15 minutes. The mixture was evaporated, re-dissolved in methanol and re-concentrated in vacuo 2 more times. The residue was purified by chromatography (silica, ethyl acetate as eluent) to give the sub-title compound (0.55 g) as a colourless oil; 1.5:1 mixture of diastereomers by NMR.

¹H NMR 400 MHz (d₆-DMSO) (major diastereomer) 8.27 (1H, s), 7.83 (1H, s), 6.56 (1H, s), 5.70 (1H, d), 4.83 (1H, m), 4.45-4.37 (1H, m), 4.00 (2H, m), 2.01-1.82 (2H, m).

c) (4S)-4-[2-(Benzoylthio)-2-(3-isoxazolyl)ethyl]-2-oxazolidinone

To a solution of triphenylphosphine (1.45 g) in THF (30 ml) at 0° C. under nitrogen was added diisopropylazodicarboxylate (1.15 ml) dropwise. After 45 minutes, a solution of thiobenzoic acid (0.77 g) and the product from step (a) (0.547 g) in THF (10 ml) was added dropwise. The reaction was warmed to room temperature and stirred for 16 h. The solvent was evaporated, and the residue purified by chromatography (silica, 2 to 75% ethyl acetate/isohexane as eluent) to give the sub-title compound (1.2 g) (1.5:1 diastereomeric mixture) as an oily solid.

¹H NMR 400 MHz (d₆-DMSO) 8.91 (1H, s), 8.02-7.53 (6H, m), 6.71 (1H, s), 5.08 (1H, dd), 4.33 (1H, t), 4.01 (1H, dd), 3.76 (1H, quintet), 2.34 (1H, m), 2.17 (1H, m).

d) 2-[[1-(3-Isoxazolyl)-2-[(4S)-2-oxooxazolidinyl]ethyl]thio]-6-methyl-3-pyridinecarbonitrile

The product from step (c) (0.6 g) was dissolved in 7M ammonia in methanol (8 ml), stirred at room temperature under nitrogen for 2 h and then the solvent was evaporated. The residue was dissolved in DMF (5 ml) and a mixture of caesium carbonate (0.85 g) and 2-chloro-6-methyl-3-pyridinecarbonitrile (0.2 g) added. After stirring for 3 h, ethyl acetate and water were added, and the organic layer separated. The aqueous layer was further extracted with ethyl acetate. The combined organic extracts were washed with 1M aqueous sodium hydroxide solution and brine, then dried (Na₂SO₄). The solvent was evaporated and the residue purified by chromatography (silica, 40 to 80% ethyl acetate/isohexane as eluent) to give the sub-title compound (0.15 g) (3:1 diastereomeric mixture) as an oily solid.

¹H NMR 400 MHz (d₆-DMSO) 8.92 (1H, d), 8.13 (1H, d), 8.01 (1H, bs), 7.25 (1H, d), 6.74 (1H, d), 5.45 (1H, dd), 4.30 (1H, t), 4.00 (1H, dd), 3.74 (1H, m), 2.58 (3H, s), 2.40-2.20 (2H, m).

e) 1,1-Dimethylethyl (4S)-4-[2-[(3-cyano-6-methyl-2-pyridinyl)thio]-2-(3-isoxazolyl)ethyl]-2-oxo-3-oxazolidinecarboxylate

To a solution of the product from step (d) (0.15 g) in THF (2 ml) were added sequentially triethylamine (0.10 ml), carbonic acid, (1,1-dimethylethoxy)carbonyl 1,1-dimethylethyl ester (0.15 g) and dimethylaminopyridine (13 mg), and the solution was then stirred for 16 h. Diethyl ether and water were added and the organic layer separated. The organic extract was washed with aqueous potassium hydrogensulfate solution and brine, then dried over (Na₂SO₄). The solvent was evaporated and the residue purified by chromatography (silica, 40 to 50% ethyl acetate in isohexane as eluent) to give the sub-title compound (70 mg) as a white solid.

¹H NMR 400 MHz (d₆-DMSO) (major diastereomer) 8.91 (1H, d), 8.15 (1H, d), 7.27 (1H, d), 6.74 (1H, d), 5.47 (1H, dd), 4.50-4.30 (3H, m), 2.57 (3H, s), 2.60-2.40 (2H, m), 1.44 (9H, s).

f) 1,1-Dimethylethyl [(1S)-3-[(3-cyano-6-methyl-2-pyridinyl)thio]-1-(hydroxymethyl)-3-(3-isoxazolyl)propyl]carbamate

To a solution of the product from step (e) (70 mg) in methanol (2.4 ml) was added caesium carbonate (0.01 g) and the solution stirred for 3 h. Ethyl acetate and water were added, and the organic layer was separated. The organic extract was washed with brine, dried (Na₂SO₄), evaporated and the residue purified by chromatography (silica, 50 to 60% ethyl acetate/isohexane as eluent) to give the sub-title compound (54 mg) as a white solid.

¹H NMR 400 MHz (CDCl₃) (major diastereomer) 8.38 (1H, d), 7.72 (1H, d), 7.00 (1H, d), 6.43 (1H, d), 5.42 (1H, d), 5.22 (1H, s), 3.80-3.67 (2H, m), 3.61 (1H, dt), 2.66 (3H, s), 2.54 (2H, m), 2.20 (1H, m), 1.45 (9H, s).

g) 2-[[(3S)-3-Amino-4-hydroxy-1-(3-isoxazolyl)butyl]thio]-6-methyl-3-pyridinecarbonitrile ethanedioate

The product from step (f) (60 mg) was dissolved in 4M HCl in dioxane (5 ml). After 2 h, the volatiles were removed, the residue taken up in methanol and passed through a SCX ion exchange resin eluting with methanol followed by 7M ammonia in methanol. The solvents were removed to afford the free base of the title product (50 mg). This material was taken up in acetonitrile (3 ml) and methanol (1 ml) and a solution of oxalic acid (14 mg) in diethyl ether added. The solvents were removed, ethyl acetate added, and the crystals filtered off and dried to give the title compound (30 mg) as a cream solid as an 80:20 (1R):(1S) diastereomeric mixture.

MS APCI+ve ^{m}/z 305 [M+H]⁺.

¹H NMR 400 MHz (d₆-DMSO) 8.92 (1H, d), 8.16 (1H, d), 7.99 (ca. 2H, vbs), 7.29 (1H, d), 6.68 (1H, d), 5.55 (1H, t), 3.63 (1H, dd), 3.52 (1H, dd), 3.20 (1H, bs), 2.58 (3H, s), 2.40-2.20 (2H, m).

### EXAMPLE 3

### 4-[[(1R,3S)-3-Amino-4-hydroxy-1-phenylbutyl]thio]-6-methyl-3-pyridinecarbonitrile ethanedioate

a) 1,1-Dimethylethyl (4S)-4-[(2R)-2-[(5-cyano-2-methyl-4-pyridinyl)thio]-2-phenylethyl]-2,2-dimethyl-3-oxazolidinecarboxylate

The product from Example 1 step (b) (406 mg) was treated with 7M ammonia in methanol (30 ml) and stirred at room temperature for 6 h.The solvent was evaporated, the residue dissolved in dry DMF (25 ml) and treated with 4-chloro-6-methyl-3-pyridinecarbonitrile (154 mg) followed by caesium carbonate (600 mg) under nitrogen. The reaction mixture was stirred for 24 h, poured into brine and ethyl acetate and the organic layer separated, washed with water (5 times) and then brine and dried (MgSO₄).The solvent was evaporated and the residue purified by chromatography (silica, 5% ethyl acetate/dichloromethane as eluent) to give the sub-title compound (177 mg, 42%) as a viscous oil.

MS APCI+ve ^{m}/z 454 [M+H]⁺.

b) 4-[[(1R,3S)-3-Amino-4-hydroxy-1-phenylbutyl]thio]-6-methyl-3-pyridinecarbonitrile ethanedioate

The product from Example 3 step (a) (177 mg) was stirred in 4M HCl in dioxane (5 ml) and methanol (5 ml) for 1 h. The reaction mixture was evaporated, azeotroped with ether (3 times), then treated with 1 equivalent of oxalic acid in ethanol (10 ml). The clear solution was treated with ether until complete precipitation and the solid collected by filtration, washed with ether and dried in vacuo at 40° C. for 2 h to give the title compound (76 mg, 48%) as a light brown solid.

MS APCI+ve ^{m}/z 314 [M+H]⁺.

¹H NMR 300 MHz (d₆-DMSO) 8.7 (1H, s), 7.54 (3H, m), 7.40 (2H, m), 7.31 (1H, m), 5.15 (1H, t), 3.48 (1H, dd), 3.38 (1H, m), 2.90 (1H, br m), 2.50 (3H, s), 2.30 (1H, m), 2.14 (1H, m).

### EXAMPLE 4

### 3-[[(1R,3S)-3-Amino-4-hydroxy-1-phenylbutyl]thio]-5-(trifluoromethyl)-2-pyridinecarbonitrile ethanedioate

a) 1,1-Dimethylethyl (4S)-4-[(2R)-2-[[2-cyano-5-(trifluoromethyl)-3-pyridinyl]thio]-2-phenylethyl]-2,2-dimethyl-3-oxazolidinecarboxylate

The product from Example 1 step (b) (411 mg) was stirred in 7M ammonia in methanol (30 ml) for 6 h. The solvent was evaporated, the residue dissolved in dry DMF (25 ml) and treated under nitrogen with stirring with 3-chloro-5-(trifluoromethyl)-2-pyridinecarbonitrile (210 mg) followed by caesium carbonate (610 mg). The reaction mixture was stirred under nitrogen overnight at room temperature, poured into brine and ethyl acetate, and the organic layer separated, washed with water (5 times) then brine and dried (MgSO₄). The solvent was evaporated and the residue purified by chromatography (silica, 5% ethyl acetate/isohexane as eluent) to give the sub-title compound (190 mg, 40%) as a viscous oil.

MS APCI+ve ^{m}/z 408 [M-Boc +1]⁺.

b) 3-[[(1R,3S)-3-Amino-4-hydroxy-1-phenylbutyl]thio]-5-(trifluoromethyl)-2-pyridinecarbonitrile ethanedioate

The product from Example 4 step a) (190 mg) was stirred in 4 M hydrogen chloride in dioxane (5 ml) and methanol (5 ml) for 1 h. The reaction mixture was evaporated, the residue treated with aqueous sodium bicarbonate and ethyl acetate, and the organic layer separated and dried (MgSO₄). The solvent was evaporated and the residue treated with an equivalent of oxalic acid in ethanol. The solution was evaporated and the residue treated with acetonitrile and a few drops of ether to precipitate a colourless solid which was collected by filtration, washed with ether and dried to give the title compound (133 mg, 78%).

MS APCI+ve ^{m}/z 368 [M+H]⁺.

¹H NMR 300 MHz (d₆-DMSO) 8.98 (1H, s), 8.33 (1H, s), 7.34 (5H, m), 5.04 (1H, t), 3.58 (1H, dd), 3.48 (1H, m), 3.05 (1H, m), 2.33 (1H, m), 2.18 (1H, m).

### EXAMPLE 5

### 2-[[(1R,3S)-3-Amino-4-hydroxy-1-phenylbutyl]thio]-6-(difluoromethyl)-3-pyridinecarbonitrile (E)-butenedioate

a) 6-(Difluoromethyl)-2-(methylthio)-3-pyridinecarbonitrile

To a solution of 6-formyl-2-(methylthio)-3-pyridinecarbonitrile (1 g) in dichloromethane under nitrogen was added [bis(methoxyethyl)amino]sulfur trifluoride (2 ml) followed by ethanol (0.05 ml). After 16 h, the reaction mixture was cautiously poured into saturated aqueous sodium bicarbonate solution. The organic layer was separated and the aqueous layer extracted with further dichloromethane. The combined organic layers were dried ((sodium sulphate)) and the solvent removed. The residue was taken up in methanol and passed through a SCX ion exchange resin eluting with methanol. The solvents were removed to afford the title product (1.2 g) as a yellow solid.

¹H NMR 400 MHz (CDCl₃) 7.93 (1H, d), 7.38 (1H, d), 6.59 (1H, t), 2.65 (3H, s).

b) 6-(Difluoromethyl)-2-(methylsulfonyl)-3-pyridinecarbonitrile

To a solution of the product from Example 5 step (a) (1.2 g) in dichloromethane (12 ml) at 0° C. was added 3-chloroperoxybenzoic acid (6.8 g of minimum 57% purity). The reaction was warmed to room temperature and stirred for 2 h. The reaction was washed with aqueous sodium bicarbonate solution and dried over (Na₂SO₄). The solvent was evaporated and the residue taken up in diethyl ether. The organic solution was washed with aqueous sodium metabisulfite solution, ice cold aqueous 0.5M sodium hydroxide solution, brine, and then dried (Na₂SO₄). The solvent was removed to give the sub-title compound (0.58 g) as a pale yellow oil.

¹H NMR 400 MHz (CDCl₃) 8.44 (1H, d), 8.03 (1H, d), 6.72 (1H, t), 3.42 (3H, s).

c) 1,1-Dimethylethyl (4S)-4-[[(2R)-2-[[3-cyano-6-(difluoromethyl)-2-pyridinyl]thio]-2-phenylethyl]-2,2-dimethyl-3-oxazolidinecarboxylate

The title compound was prepared by the method of Example 4 step (a) using the product of Example 1 step (b) and 6-(difluoromethyl)-2-(methylsulphonyl)-3-pyridinecarbonitrile to give, after purification by chromatography (silica, 5% ethyl acetate in isohexane as eluent) the sub-title compound (252 mg, 74%) as a viscous oil.

MS APCI+ve ^{m}/z 390 [M-Boc+1]⁺.

d) 2-[[(1R,3S)-3-Amino-4-hydroxy-1-phenylbutyl]thio]-6-(difluoromethyl)-3-pyridinecarbonitrile (E)-butenedioate

The product from step (c) was deprotected as in Example 4 step (b) and then converted into the (E)-butenedioate salt by addition of one equivalent of fumaric acid to give the title compound (121 mg, 51%) as a colourless foam.

MS APCI+ve ^{m}/z 350 [M+H]⁺.

¹H NMR 300 MHz (d₆-DMSO) 8.40 (1H, d), 7.59 (1H, d), 7.52 (2H, d), 7.31 (3H, m), 7.10 (1H, t), 6.45 (2H, s), 5.35 (1H, q), 3.38 (2H, m), 2.75 (1H, br m), 2.31 (1H, m), 2.18 (1H, m).

### EXAMPLE 6

### 2-[[(1R,3S)-3-Amino-4-hydroxy-1-phenylbutyl]thio]-6-(fluoromethyl)-3-pyridinecarbonitrile (E)-butenedioate

a) 6-(Fluoromethyl)-2-(methylthio)-3-pyridinecarbonitrile

To a solution of 6-formyl-2-(methylthio)-3-pyridinecarbonitrile (1 g) in ethanol (12 ml) was added sodium borohydride (0.212 g). After 2 h, the volatiles were removed and ethyl acetate and water added. The organic layer was separated and the aqueous layer extracted with further ethyl acetate. The combined organic layers were dried (Na₂SO₄) and the solvent removed to afford 6-(hydroxymethyl)-2-(methylthio)-3-pyridinecarbonitrile (1 g) as yellow solid. This material was taken up in dichloromethane (10 ml) under nitrogen and [bis(methoxyethyl)amino]sulfur trifluoride (1 ml) in dichloromethane (3 ml) was added. After 16 h the reaction mixture was cautiously poured into saturated aqueous sodium bicarbonate solution. The organic layer was separated, dried (Na₂SO₄) and the solvent removed. The residue was taken up in methanol and passed through a SCX ion exchange resin eluting with methanol. The solvents were removed to afford the sub-title product (0.88 g) as a yellow solid.

¹H NMR 400 MHz (CDCl₃) 7.85 (1H, d), 7.23 (1H, d), 5.48 (2H, d), 2.60 (3H, s).

b) 6-(Fluoromethyl)-2-(methylsulfonyl)-3-pyridinecarbonitrile

The title compound was prepared by the method of Example 5 step (b) using the product of Example 6 step (a) and 3-chloroperoxybenzoic acid. The product was obtained as a pale green oil which solidified upon standing.

¹H NMR 400 MHz (CDCl₃) 8.33 (1H, d), 7.87 (1H, d), 5.60 (2H, d), 3.37 (3H, s).

c) 1,1-Dimethylethyl (4S)-4-[[(2R)-2-[[3-cyano-6-(fluoromethyl)-2-pyridinyl]thio]-2-phenylethyl]-2,2-dimethyl-3-oxazolidinecarboxylate

The title compound was prepared by the method of Example 4 step (a) using the product of Example 1 step (b) and 6-(fluoromethyl)-2-(methylsulphonyl)-3-pyridinecarbonitrile to give, after chromatography (silica, 10 to 30% diethyl ether in isohexane as eluent) the sub-title compound (318 mg) as an off white foam.

¹H NMR 400 MHz (d₆-DMSO) 8.26 (1H, d), 7.46 (2H, d), 7.35 (3H, m), 7.25 (1H, t), 5.76-5.44 (2H, m), 5.14 (1H, dd), 4.00-3.53 (3H, br m), 2.50-2.00 (2H, m), 1.46-1.36 (15H, m).

d) 2-[[(1R,3S)-3-Amino-4-hydroxy-1p-henylbutyl]thio]-6-(fluoromethyl)-3-pyridinecarbonitrile (E)-butenedioate

The product from step (c) was deprotected as in Example 4 step (b) and then converted into the (E)-butenedioate salt by addition of one equivalent of fumaric acid to give the title compound (224 mg) as an off white foam.

MS APCI+ve ^{m}/z 332 [M+H]⁺.

¹H NMR 400 MHz (CD₃OD) 8.07 (1H, d), 7.49 (2H, m), 7.38-7.27 (4H, m), 6.68 (2H, s), 5.62 (1H, q), 5.49 (1H, t), 3.69 (1H, dd), 3.55 (1H, dd), 3.26 (1H, m), 2.43 (1H, ddd), 2.34 (1H, ddd).

### EXAMPLE 7

### 2-[[(1R,3S)-3-Amino-4-hydroxy-1-(3-pydridinyl)butyl]oxy]-4-chloro-5-fluorobenzonitrile Dihydrochloride

a) 1,1-Dimethylethyl (4S)-4-[(2R)-2-hydroxy-2-(3-pyridinyl)ethyl]-2,2-dimethyl-3-oxazolidinecarboxylate

The title compound was prepared by the method of Example 1 step (a) to give the more polar diastereomer as a colourless oil.

MS APCI+ve ^{m}/z 222 [M+H-Boc]⁺.

b) 1,1-Dimethylethyl (4S)-4-[(2R)-2-(5-chloro-2-cyano-4-fluorophenoxy)-2-(3-pyridinyl)ethyl]-2,2-dimethyl-3-oxazolidinecarboxylate

Sodium hydride (60% in mineral oil) (24 mg) was added cautiously to a stirred solution of 4-chloro-2,5-difluorobenzonitrile (90 mg) and the product from step (a) (165 mg) in dry DMF (5 ml) and stirring was continued for 2 h. The reaction mixture was quenched with water, extracted twice with ethyl acetate, the extracts dried (Na₂SO₄) and evaporated. The residue was purified by chromatography (silica, 10% ethyl acetate/hexanes as eluent) to give the sub-title compound (220 mg) as a colourless foam.

MS APCI+ve ^{m}/z 376 [M+H-Boc]⁺.

c) 2-[[(1R,3S)-3-Amino-4-hydroxy-1-(3-pyridinyl)butyl]oxy]-4-chloro-5-fluorobenzonitrile dihydrochioride

The product from step (b) (220 mg) was stirred with methanol (1 ml) and 4 M hydrogen chloride in dioxane (2 ml) for 2 h. The reaction mixture was evaporated and triturated with diethyl ether to give the title compound (130 mg) as a white solid.

MS APCI+ve ^{m}/z 336 [M+H]⁺.

¹H NMR 400 MHz (d₆-DMSO) 8.95 (1H, s), 8.75 (1H, d), 8.27-8.21 (4H, m), 8.06 (1H, d), 7.81-7.78 (1H, t), 7.62 (1H, d), 6.23-6.20 (1H, m), 3.72-3.65 (1H, dd), 3.61-3.58 (1H, m), 3.3-3.2 (1H, br.s), 2.40-2.31 (1H, m), 2.27-2.20 (1H, m).

### EXAMPLE 8

### 2-[[(1R,3S)-3-Amino-4-hydroxy-1-(2-thiazolyl)butyl]oxy]-4-chloro-5-fluorobenzonitrile ethanedioate

a) 1,1-Dimethylethyl (4S)-4-[(2S)-2-hydroxy-2-(2-thiazolyl)ethyl]-2,2-dimethyl-3-oxazolidinecarboxylate and 1,1-Dimethylethyl (4S)-4-[(2R)-2-hydroxy-2-(2-thiazolyl)ethyl]-2,2-dimethyl-3-oxazolidinecarboxylate

To a stirred solution of (4S)-2,2-dimethyl-4-(2-oxoethyl)-3-oxazolidinecarboxylic acid 1,1-dimethylethyl ester (10.75 g) in dry dichloromethane (225 ml) at room temperature and under nitrogen, was added 2-(trimethylsilyl)thiazole (10.6 ml). The mixture was then stirred at room temperature for 18 h. The reaction mixture was evaporated to dryness and the residues dissolved in THF (27 ml) and tetrabutylammonium fluoride (1.0M in THF, 6 ml) added. The mixture was then stirred at room temperature for 2 h. The resultant mixture was evaporated to dryness, water (80 ml) added, and the mixture was extracted with dichloromethane four times. The combined organic extracts were washed with brine, dried (MgSO₄) and concentrated to an oil. The crude mixture of diastereomers was purified by chromatography (silica, 20 to 60% ethyl acetate/isohexane as eluent) to give the (4S, 2S) isomer (7.6 g) as a pale yellow oil.

MS APCI+ve ^{m}/z 329 [M+H]⁺.

¹H NMR 400 MHz (CDCl₃) 7.71 (1H, d), 7.28 (1H, d), 5.14 (1H, m), 5.07 (1H, m), 4.20 (1H, m), 4.05 (1H, m), 3.85 (1H, m), 2.20-2.50 (2H, m), 1.48 (15H, m).

Further elution gave the (4S, 2R) isomer (6.4 g) as a colourless solid.

MS APCI+ve ^{m}/z 329 [M+H]⁺.

¹H NMR 400 MHz (CDCl₃) 7.72 (1H, d), 7.28 (1H, d), 5.68 (1H, d), 4.94 (1H, m), 4.35 (1H, m), 4.04 (1H, m), 3.71 (1H, d), 2.42 (1H, m), 1.90 (1H, m), 1.62 (3H, s), 1.53 (3H, s), 1.51 (9H, s).

b) 1,1-Dimethylethyl (4S)-4-[(2R)-2-(5-chloro-2-cyano-4-fluorophenoxy)-2-(2-thiazolyl)ethyl]-2,2-dimethyl-3-oxazolidinecarboxylate

To a solution of the (4S, 2R) isomer from step(a) (3 g) and 4-chloro-2,5-difluorobenzonitrile (1.59 g) in dry THF (100 ml) containing dry DMF (10 ml) at room temperature was added sodium hydride (60% in oil, 385 mg). After the addition was complete the mixture was stirred for 18 h and then poured into water (60 ml) and extracted with diethyl ether (3 times). The combined organic extracts were washed with brine and dried (MgSO₄). The mixture was evaporated to dryness to give an oil which was purified on silica gel eluting with 20 to 25% ethyl acetate in isohexane. The title compound was isolated as a yellow coloured oil (4.0 g, 91%).

MS APCI+ve ^{m}/z 482/4 [M+H]⁺.

c) 2-[[(1R,3S)-3-Amino-4-hydroxy-1-(2-thiazolyl)butyl]oxy]-4-chloro-5-fluorobenzonitrile ethanedioate

To a solution of the product from step (b) (4.0 g) in methanol (100 ml) was added a solution of 4M HCl in dioxane. The mixture was stirred at 20° C. for 1.5 h, then evaporated to dryness. The residue was dissolved in aqueous sodium bicarbonate solution and extracted with ethyl acetate (four times). The combined extracts were washed with brine, dried (MgSO₄) and purified by chromatography (silica, ethyl acetate, then 10% (7M ammonia in methanol) in dichloromethane as eluents) to give a mixture which was concentrated and dissolved in a mixture of ethanol and acetonitrile. A solution of oxalic acid (730 mg) in diethyl ether was added and the resultant mixture was evaporated to dryness then recrystallised from a mixture of ethanol, acetonitrile and diethyl ether to give the title compound (2.14 g) as a white solid.

MS APCI+ve ^{m}/z 342/4 [M+H]⁺.

¹H NMR 400 MHz (d₆-DMSO) 8.07 (1H, d), 7.89 (1H, d), 7.84 (1H, d), 7.70 (1H, d), 6.24 (1H, m), 3.67 (1H, m), 3.55 (1H, m), 3.29 (1H, m), 2.30-2.44 (2H, m).

### EXAMPLE 9

### 2-[[(1R,3S)-3-Amino-4-hydroxy-1-(5-isothiazolyl)butyl]oxy]-4-chloro-5-fluorobenzonitrile Hydrochloride

a) 1,1-Dimethylethyl (4S)-4-[(2S)-2-hydroxy-2-(5-isothiazolyl)ethyl]-2,2-dimethyl-3-oxazolidinecarboxylate and 1,1-Dimethylethyl (4S)-4-[(2R)-2-hydroxy-2-(5-isothiazolyl)ethyl]-2,2-dimethyl-3-oxazolidinecarboxylate

A solution of isothiazole (1.42 g) in dry THF (50 ml) under a nitrogen atmosphere was cooled to −78° C. and butyl lithium (1.6M in hexanes, 10.3 ml) added dropwise keeping the temperature below −70° C. The resulting red solution was stirred at −78° C. for 1 h, then a solution of 1,1-dimethylethyl (4S)-2,2-dimethyl-4-(2-oxoethyl)-3-oxazolidinecarboxylate (4 g) in dry THF (20 ml) was added over 5 minutes. After the addition was complete the cooling was removed and the mixture stirred for 30 minutes. The reaction mixture was poured into water (150 ml) and the products extracted into diethyl ether (2×150 ml). The combined extracts were dried (MgSO₄) and concentrated to an oil. Purification by chromatography (silica, 50% isohexane in diethyl ether as eluent) gave the (4S, 2S) sub-title compound (600 mg) as a colourless oil.

MS APCI+ve ^{m}/z 329 [M+H]⁺.

Further elution gave the (4S, 2R) sub-title compound (500 mg) as a colourless oil.

MS APCI+ve ^{m}/z 329 [M+H]⁺.

b) 2-[[(1R,3S)-3-Amino-4-hydroxy-1-(5-isothiazolyl)butyl]oxy]-4-chloro-5-fluorobenzonitrile Hydrochloride

A solution of the (4S, 2R) isomer from step (a) (500 mg) in a mixture of dry THF (20 ml) and dry DMF (2 ml) was treated with 4-chloro-2,5-difluorobenzonitrile (416 mg). To this mixture under nitrogen was added sodium hydride (60% dispersion in mineral oil, 91 mg). The mixture was then stirred for 3 h at 20° C. The reaction mixture was poured into water (100 ml), and the products extracted into diethyl ether (2×100 ml). The combined extracts were dried over (MgSO₄) and concentrated to an oil. The major product was isolated by column chromatography on silica gel (25% diethyl ether/isohexane as eluent) and dissolved in methanol (5 ml). The solution was treated with 4M HCl in dioxane (2 ml) and stirred for 2 h. Concentration of the solution to dryness and trituation with acetonitrile afforded the title compound (190 mg) as a colourless solid.

MS APCI+ve ^{m}/z 342 [M+H]⁺.

¹H NMR 400 MHz (d₆-DMSO) 8.57 (1H, s), 8.07 (1H, d), 8.1 (3H, br s), 7.67 (1H, d), 7.54 (1H, s), 6.5 (1H, dd), 5.41 (1H, t), 3.7-3.5 (2H, m), 3.25 (1H, br m), 2.4-2.2 (2H, m).

### EXAMPLE 10

### 4-[[(1R,3S)-3-Amino-4-hydroxy-1-phenylbutyl-]thio]-6-methoxy-3-pyridinecarbonitrile (E)-butenedioate

a) Phenylmethyl (3S)-3-[[(1,1-dimethylethoxy)carbonyl]amino]-4-hydroxy-butanoate

A solution of 4-(phenylmethyl) N-[(1,1-dimethylethoxy)carbonyl]-1-(2,5-dioxo-1-pyrrolidinyl)) L-aspartate (75.0 g) in THF (200 ml) was added over 1 h to a suspension of sodium borohydride (6.84 g) in THF (60 ml) and water (90 ml) at −5° C. (internal temperature kept below 15° C.). Further sodium borohydride (6.8 g in two batches) was added and stirred for 45 min. The mixture was poured into cold, stirred, half-saturated ammonium chloride solution (600 ml) and extracted with ethyl acetate (twice). The organic layers were dried (MgSO₄) and evaporated to give the sub-title compound as a waxy solid (56.24 g).

MS APCI+ve ^{m}/z 210 [M+H-BOC]⁺.

¹H NMR 300 MHz (CDCl₃) 7.41-7.27 (5H, m), 5.24-5.10 (3H, m), 4.15-3.96 (1H, m), 3.71 (2H, d), 2.69 (2H, d), 1.44 (9H, s).

b) Phenylmethyl (4S)-3-[(1,1-dimethylethoxy)carbonyl]-2,2-dimethyl-4-oxazolidineacetate

2-Methoxypropene (46 ml) was added over 20 min to a solution of the product from step a) (74.88 g) 2,2-dimethoxypropane (30 ml) and p-toluenesulfonic acid (1.21 g) in dichloromethane (300 ml) at 0° C. and stirred at 0° C. for 1 h and at 20° C. for 1 h. 1M NaHCO₃ was added and the mixture was extracted with dichloromethane (3×200 ml). The organic layers were dried (MgSO₄) and evaporated to give a colourless oil which was dissolved in toluene (300 ml), 2,2-dimethoxypropane (45 ml) and p-toluenesulfonic acid (1.2 g) added and the mixture was heated at 80° C. for 2 h. On cooling, K₂CO₃ was added and the mixture was extracted with ethyl acetate (twice). The organic layers were dried (MgSO₄) and evaporated to give the sub-title compound (83.8 g) as a pale yellow oil.

¹H NMR 300 MHz (CDCl₃) 7.36-7.28 (5H, m), 5.12 (2H, d), 4.38-3.97 (2H, m), 3.84 (1H, d), 3.05-2.48 (2H, m), 1.62-1.50 (6H, m), 1.46 (9H, s).

c) (4S)-3-[(1,1-Dimethylethoxy)carbonyl]-2,2-dimethyl-4-oxazolidineacetic acid

A suspension of palladium on carbon (10%, 3.8 g) and the product from step b) (83.8 g) in ethanol (250 ml) was stirred under hydrogen (4 atmospheres pressure) for 3.5 h (5.3 l hydrogen uptake). The mixture was filtered through celite and evaporated. Ethyl acetate (100 ml) and 1M K₂CO₃ (200 ml) were added and the organic layer was separated and further extracted with 1M K₂CO₃ (40 ml) and 1M NaHCO₃ (40 ml). The aqueous layers were washed with ethyl acetate, combined and acidified at 0° C. by dropwise addition of 4M HCl (130 ml). The aqueous was extracted with ethyl acetate (3×200 ml) and the organic layers were dried (MgSO₄) and evaporated to give the sub-title compound as a pale orange gum (56.24 g), which slowly crystallised.

¹H NMR 300 MHz (CDCl₃) 4.33-4.12 (1H, m), 4.09-4.00 (1H, m), 3.86 (1H, d), 3.02-2.77 (1H, m), 2.62-2.50 (1H, m), 1.62-1.54 (6H, m), 1.53 (9H, s).

d) 1,1-Dimethylethyl (4S)-4-[2-(methoxymethylamino)-2-oxoethyl]-2,2-dimethyl-3-oxazolidinecarboxylate

N,O,-Dimethylhydroxylamine hydrochloride (21.4 g), EDCI (41.94 g), N-methylmorpholine (24 ml) and DMAP (26.4 g) were added to a solution of the product from step c) (59.2 g) in CH₂Cl₂ (400 ml) at 0° C. and then stirred at 20° C. for 18 h. 2M HCl (200 ml) was added, the organic layer was separated and the aqueous was further extracted twice. The organic layers were washed with 2M HCl (50 ml) and NaHCO₃ (2×100 ml), combined, dried (MgSO₄) and evaporated to give the sub-title compound (60.2 g).

MS APCI+ve ^{m}/z 303 [M+H]⁺.

¹H NMR 300 MHz (CDCl₃) 4.38-4.19 (1H, m), 4.08 (1H, dd), 3.87 (1H, t), 3.70 (3H, s), 3.17 (3H, s), 3.07-2.45 (2H, m), 1.63-1.42 (15H, m).

e) 1,1-Dimethylethyl (4S) 2,2-dimethyl-4-(2-oxo-2-phenylethyl)-3-oxazolidinecarboxylate

Phenyl magnesium bromide (231 ml, 1M in THF) was added over 15 min to a solution of the product from step d) (60.1 g) in THF (360 ml) at −10 to −5° C. and stirred for 2 h. Further phenyl magnesium bromide (7 ml, 3M in ether) was added and stirred at 0° C. for 1 h then quenched by the addition of saturated NH₄Cl (250 ml) and 2M HCl (150 ml). The mixture was extracted with ethyl acetate (thrice) and the organic layers were washed with brine, combined, dried (MgSO₄) and evaporated to give the sub-title compound (64.8 g) as an off-white solid.

¹H NMR 300 MHz (CDCl₃) 7.98 (2H, d), 7.64-7.40 (3H, m), 4.50-4.35 (1H, m), 4.15-4.05 (1H, m), 3.88-3.65 (2H, m), 3.49-3.36 and 3.25-3.01 (1H, m), 1.70-1.35 (15H, m).

f) 1,1-Dimethylethyl (4S)-4-[(2S)-2-hydroxy-2-phenylethyl]-2,2-dimethyl-3-oxazolidinecarboxylate

Borane (176 ml, 1M in THF) was added to a solution of (R) methyl-CBS-oxazaborolidine (16 ml, 1M in toluene) in THF (20 ml) and cooled to −20° C. A solution of the product from step e) (64.6 g) in THF (200 ml) was added over 1.5 h, keeping the internal temperature below −15° C., and then stirring at this temperature for 22 h. Methanol (40 ml) was added slowly and the solution was evaporated and then azeotroped with methanol to give a pale yellow oil (69 g). Ether and 1M KHSO₄ (20 ml) were added and the mixture was filtered and evaporated. Purification by chromatography (silica, 40-60 petrol/ether as eluent) gave the sub-title compound (37.4 g) as a white solid, identical with the major product from Example 1 step a).

Further elution gave the (4S, 2R) isomer as a white solid (20.0 g) identical with the minor product from Example 1 step a).

g) 1,1-Dimethylethyl (4S) 4-[(2R)-2-(benzoylthio)-2-phenylethyl]-2,2-dimethyl-3-oxazolidinecarboxylate

Diisopropyl azodicarboxylate (21.5 ml) in THF (20 ml) was added dropwise to a solution of triphenylphosphine (28.73 g) in THF (230 ml) at −10° C. and the white suspension was stirred for 30 min. A solution of the product from step f) (17.58 g) and thiobenzoic acid (12.8 ml) in THF (100 ml) was added over 45 min at −10° C. and then stirred at −10° C. to 4° C. for 18 h. The solvent was removed in vacuo, ether added and stirred until a precipitate formed. The mixture was filtered and the solids washed with isohexane/ether (1:1). The solution was washed with aqueous NaHCO₃ and the aqueous layer extracted with ether. The combined organic layers were dried (MgSO₄), evaporated and purified by chromatography (silica, 40-60 petrol/dichloromethane (1:1 then 0:1) as eluent) to give a solid. This was crystallised from isohexane at −78° C. to give the sub-title compound (14.76 g) as a white solid, identical with the major product from Example 1 step b).

¹H NMR 300 MHz (CDCl₃) 7.93 (2H, d), 7.61-7.21 (8H, m), 4.79 (1H, dt), 4.18-3.83 (3H, m), 2.64-2.35 (1H, m), 2.23-2.09 (1H, m), 1.62-1.41 (15H, m).

h) 2-Chloro-5-(4,5-dihydro-4,4-dimethyl-2-oxazolyl)-pyridine

A suspension of 2-chloropyridine-4-carboxylic acid (100 g) in thionyl chloride (370 ml) was heated at 80° C. for 4 h. The reaction mixture was evaporated in vacuo, the residue azeotroped with toluene and then taken up into dichloromethane (300 ml) The solution was added dropwise over 1 h at 0° C. to a solution of 2-amino-2-methylpropanol (118.8 g) in dichloromethane (300 ml) and then stirred at 20° C. for 16 h. Water (500 ml) was added and the mixture was extracted with dichloromethane (5×500 ml). A solid suspension, which formed during extraction, is the required intermediate amide and needs extensive extraction. The organic layer was dried (MgSO₄) and evaporated to leave the intermediate amide (125.5 g).

This was suspended in dichloromethane (200 ml) at 0° C. and thionyl chloride (100 ml) was added dropwise and stirred for 1 h. A thick precipitate formed and more dichloromethane (300 ml) is added and reaction stirred for a further hour. The solvent was removed in vacuo to give the product as the hydrochloride salt (120 g).

¹H NMR 300 MHz (CD₃OD) 9.03 (1H, t), 8.42 (1H, dd), 7.80(1H, dd), 4.96 (2H, s), 1.68 (6H, s).

The solid was suspended in water (800 ml) and treated with solid NaHCO₃ (ca. 70 g portion-wise) until gas evolution ceased. The mixture was extracted with dichloromethane (2×500 ml), dried (MgSO₄) and evaporated to give the sub-title compound (99.5 g).

¹H NMR (CDCl₃) 8.90 (1H, dd), 8.17 (1H, dd), 7.37 (1H, dd), 4.14 (2H, s), 1.39 (6H, s).

i) 5-(4,5-Dihydro-4,4-dimethyl-2-oxazolyl)-2-methoxy-pyridine

The product from step h) (99.5 g) in methanol (500 ml) was treated with sodium methoxide (0.61 mol of a 25 wt % solution in methanol) and heated at reflux for 12 hrs. The solvent was removed under reduced pressure and the residue taken up in water (200 ml) and extracted with dichloromethane (2×300 ml). The extract was dried (MgSO₄) evaporated to dryness to give the sub-title compound as an orange oil (85 g).

¹H NMR 300 MHz (CDCl₃) 8.68 (1H, dd), 8.10 (1H, dd), 6.75 (1H, dd), 4.09 (2H, s), 3.98 (3H, s), 1.37 (6H, s).

j) 5-(4,5-Dihydro-4,4-dimethyl-2-oxazolyl)-2-methoxy-4-(methylthio)-pyridine

To 2,2,6,6-tetramethylpiperidine (51.22 g) in THF, under nitrogen, at 0° C., was added n-BuLi (227 ml of 1.6M solution in hexanes) dropwise and stirred for 15 min. The reaction mixture was cooled to −78° C. and the product from step i) (43.97 g) in THF (50 ml) was added dropwise. The cooling bath was removed and the reaction temperature was allowed to warm up to −20° C. and kept at this temperature for 30 min. It was then cooled to −78° C. and dimethyldisulphide (80 ml) was dripped in. The reaction mixture temperature rose to −30° C. during this addition. The cooling bath was then removed and the reaction was stirred to room temperature for 12 h. The resulting red solution was quenched with water and concentrated to ca. 600 ml on a rotary evaporator. Water was added (500 ml) and the mixture was extracted with ethyl acetate (2×600 ml). The combined organics were washed with citric acid (500 ml of 1M aqueous solution), dried (MgSO₄) and evaporated to give the sub-title compound as a pale yellow solid, (58.5 g).

¹H NMR 300 MHz (CDCl₃) 8.50 (1H, s), 6.52 (1H, s), 4.04 (2H, s), 3.97 (3H, s), 2.40 (3H, s), 1.40 (6H, s).

k) 6-Methoxy-4-(methylthio)-3-pyridinecarbonitrile

A stirred solution of the oxazoline from step j) (45 g) in pyridine (350 ml) was treated with phosphorus oxychloride (68 ml) and the mixture stirred under reflux for 4.5 h. The dark brown solution was cooled to room temperature and cautiously poured onto ice (1 kg). The resulting suspension was filtered and the solid washed with water (300 ml), 2M HCl (100 ml) and again with water (300 ml). The damp product was dissolved in dichloromethane (600 ml) and the solution dried (MgSO₄). Activated charcoal was added (15 g) and the suspension filtered. Concentration of the filtrate and trituration of the solid with 40-60 petrol gave the sub-title compound as a very pale pink solid (26 g).

¹H NMR 300 MHz (CDCl₃) 8.31 (1H, s), 6.51 (1H, s), 3.98 (3H, s), 2.52 (3H, s).

1) 6-Methoxy-4-(methylsulfonyl)-3-pyridinecarbonitrile

A solution of the product from step k) (13 g) in dichloromethane (150 ml) was cooled to 0° C. and treated with portion-wise addition of MCPBA (21.74 g of ˜57% purity) over 10 min. The mixture was allowed to slowly warm up to 20° C. After 8 hrs LC/MS indicated a mixture of sulphoxide/sulphone products (72:28). Additional MCPBA was added (7.2 g) and after a further 4 hrs LC/MS indicated a 50:50 mixture of products. More MCPBA was added (12 g) and stirring continued for a further 2 h before reaction was complete. The reaction was cooled to 0° C. and treated with excess aqueous sodium metabisulphite solution. The organic layer was washed with sat. NaHCO₃ (3×200 ml), dried (MgSO₄) and evaporated to give the sub-title compound as a white solid (11.2 g).

¹H NMR³⁰⁰ MHz (CDCl₃) 8.69 (1H, s), 7.47 (1H, s), 4.09 (3H, s), 3.28 (3H, s).

m) 1,1-Dimethylethyl (4S)-4-[(2R)-2-[(5-cyano-2-methoxy-4-pyridinyl)thio]-2-phenylethyl]-2,2-dimethyl-3-oxazolidinecarboxylate

A solution of the product from step g) (10.0 g) in methanol (75 ml) at 20° C. was treated with 7 M ammonia in methanol (50 ml) every hour for eight hours. The methanol was evaporated and the residue was dissolved in dry DMF (100 ml). The product from step 1) (4.8 g) was added and allowed to dissolve, followed by caesium carbonate (7.38 g) and the mixture was stirred at 20° C. for 18 h. Ethyl acetate (200 ml) and water (200 ml) were added and the aqueous layer was separated and washed with ethyl acetate (2×100 ml). The combined ethyl acetate layers were washed with water (3×200 ml) and brine, dried (MgSO₄), filtered and concentrated in vacuo to leave a crude yellow gum. Purification by chromatography (silica, 30% ethyl acetate in isohexane as eluent) gave the sub-title compound as a translucent foam (7.4 g).

MS APCI+ve ^{m}/z 470 ([M(+H)]⁺).

¹H 300 MHz (CDCl₃) 8.51 (1H, s), 7.56 (2H, d), 7.37 (2H, t), 7.27 (1H, t), 6.87-6.83 (1H, m), 4.98-4.84 (1H, m), 4.14-3.60 (3H, m), 3.85 (3H, s), 2.30-1.85 (2H, m), 1.49-1.38 (15H, s).

n) 4-[[(1R,3S)-3-Amino-4-hydroxy-1-phenylbutyl]thio]-6-methoxy-3-pyridinecarbonitrile (E)-butenedioate

To a solution of the product from step m) (7.1 g) in methanol (100 ml) at 0° C., was added 4M HCl in dioxane (100 ml). The mixture was stirred at 20° C. for 2 h and the solvent was removed in vacuo. The residue was partitioned between aqueous sodium bicarbonate (200 ml) and dichloromethane (200 ml). The aqueous phase was extracted with dichloromethane (2×100 ml) and the combined extracts were dried (MgSO₄), filtered and concentrated in vacuo to give the title compound free base as a pale yellow oil (4.8 g).

MS (APCI+ve) ^{m}/z 330 [M(+H)]⁺.

¹H 300 MHz (CDCl₃) 8.27 (1H, s), 7.43 (2H, d), 7.34 (2H, t), 7.27 (1H, t), 6.65 (1H, s), 4.75 (1H, dd), 3.90 (3H, s), 3.51-3.27 (2H, m), 2.71-2.63 (1H, m), 2.12-1.88 (2H, m).

A solution of this compound in methanol (50 ml) was added to a solution of fumaric acid (1.6 g) in methanol (50 ml) and stirred at 20° C. The solvent was removed in vacuo and the residue was triturated with acetonitrile. The precipitate was collected and washed with acetonitrile, and dried to give the title compound as a white solid (5.1 g), m.p. 172-173° C.

MS (APCI+ve) ^{m}/z 330 [M(+H)]⁺.

¹H 500 MHz (DMSO-d₆) 8.53 (1H, s), 7.55 (2H, d), 7.39 (2H, t), 7.30 (1H, t), 7.00 (1H, s), 6.45 (2H, s), 5.15 (1H, dd), 3.89 (3H, s), 3.38 (2H, ddd), 2.73-2.65 (1H, m), 2.25-2.01 (2H, m).

### EXAMPLE 11

### 4-[[(1R,3R)-3-Amino-4-hydroxy-1-phenylbutyl]thio]-6-methoxy-3-pyridinecarbonitrile (E)-butenedioate

a) 1,1-Dimethylethyl (4R)-4-(2-oxo-2-phenylethyl)-2,2-dimethyl-3-oxazolidinecarboxylate

The sub-title compound was prepared from 4-(phenylmethyl) N-[(1,1-dimethylethoxy)carbonyl]-D-aspartate, the enantiomer of the starting material in Example 10 step a), by the methods of Example 10 steps a) to e).

MS APCI+ve ^{m}/z 320 [M+H]⁺.

¹H NMR 300 MHz (d₆-DMSO) 7.98 (2H, d), 7.61-6.83 (3H, m), 4.69 (1H, bs), 4.10 (1H, t), 3.83-3.68 (2H, bm), 3.15 (1H, m), 1.66-1.42 (15H, m).

b) 1,1-Dimethylethyl (4R)-4-[(2R)-2-(benzoylthio)-2-phenylethyl]-2,2-dimethyl-3-oxazolidinecarboxylate

The sub-title compound was prepared by the methods of Example 10 steps f) and g) from the product from step a). The chiral reduction (step f) was carried out using (R) methyl-CBS-oxazaborolidine.

MS APCI+ve ^{m}/z 342 [M+H]⁺.

¹H NMR 400 MHz (d₆-DMSO) 7.86 (2H, d), 7.85-7.24 (8H, m), 4.77 (1H, m), 4.01-3.87 (2H, m), 3.62 (1H, bs), 2.16 (2H, m), 1.47-1.36 (15H, m).

c) 4-[[(1R,3R)-3-Amino-4-hydroxy-1-phenylbutyl-]thio]-6-methoxy-3-pyridinecarbonitrile, (E)-butenedioate.

The title product was prepared by the methods of Example 10 steps m) to n). M.p. 221-223° C.

MS APCI+ve ^{m}/z 330 [M+H]⁺.

¹H NMR 400 MHz (d₆-DMSO) (90° C.) 8.54 (1H, s), 7.54 (2H, d), 7.39 (2H, t), 7.30 (1H, t), 6.87 (1H, m), 6.45 (2H, m), 5.09 (1H, m), 3.88 (3H, s), 3.61-3.55 (2H, m), 2.88 (1H, m), 2.33-2.09 (2H, m).

### EXAMPLE 12

### 4-[[(1S,3R)-3-Amino-4-hydroxy-1-phenylbutyl]thio]-6-methoxy-3-pyridinecarbonitrile, (E)-butenedioate

a) 1,1-Dimethylethyl (4R)-4-[(2S)-2-(benzoylthio)-2-phenylethyl]-2,2-dimethyl-3-oxazolidinecarboxylate.

The sub-title compound was prepared by using (S) methyl-CBS-oxazaborolidine catalyst in the chiral reduction of the product from Example 11 step a) following the procedure of Example 10 steps f) to g).

MS APCI+ve ^{m}/z 342 [M+H]⁺.

¹H NMR 400 MHz (d₆-DMSO) 7.85 (2H, d), 7.63 (1H, t), 7.50 (2H, t), 7.42 (2H, d), 7.34 (2H, t), 7.27 (1H, t), 4.80 (1H, m), 3.95 (1H, m), 3.85-3.13 (2H, m), 2.14 (2H, m), 1.45-1.36 (15H, m).

b) 4-[[(1S,3R)-3-Amino-4-hydroxy-1-phenylbutyl]thio]-6-methoxy-3-pyridinecarbonitrile, (E)-butenedioate

The title compound was prepared by the methods of Example 10 steps m) to n) from the product of step a). M.p. 162.5-163° C.

MS APCI+ve ^{m}/z 330 [M+H]⁺.

¹H NMR 400 MHz (d₆-DMSO) 8.53 (1H, s), 7.55 (2H, d), 7.38 (2H, t), 7.30 (1H, t), 7.00 (1H, s), 6.44 (1H, s), 5.12 (1H, m), 3.89 (3H, s), 3.36-3.26 (2H, m), 2.62 (1H, m), 2.22-2.08 (1H, m), 2.01-1.95 (1H, m).

### EXAMPLE 13

### 4-[[(1S,3S)-3-Amino-4-hydroxy-1-phenylbutyl]thio]-6-methoxy-3-pyridinecarbonitrile (E)-butenedioate

a) 4-[(2S)-2-(Benzoylthio)-2-phenylethyl]-2,2-dimethyl-1,1-dimethylethyl (4S)-3-oxazolidinecarboxylate

The sub-title compound was prepared from the minor isomer of Example 1 step a), following the method of Example 10 step g).

MS APCI+ve ^{m}/z 342 [M+H]⁺.

¹H NMR 300 MHz (d₆-DMSO) 7.91 (2H, d), 7.57-7.23 (8H, m), 4.76 (1H, m), 4.17-3.61 (3H, m), 2.50-2.18 (2H, m), 1.66-1.41 (15H, m).

b) 4-[[(1S,3S)-3-Amino-4-hydroxy-1-phenylbutyl]thio]-6-methoxy-3-pyridinecarbonitrile, (E)-butenedioate

The title compound was prepared by the methods of Example 10 steps m) to n) from the product of step a). M.p. 213-228° C.

MS APCI+ve ^{m}/z 330 [M+H]⁺.

¹H NMR 400 MHz (d₆-DMSO) 8.53 (1H, s), 7.53 (2H, d), 7.39 (2H, t), 7.30 (1H, t), 7.96 (1H, s), 6.43 (2H, s), 5.08 (1H, t), 3.88 (3H, s), 3.58 (2H, m), 2.86 (1H, bs), 2.25-2.28 (1H, m), 2.08 (1H, m).

### EXAMPLE 14

### 4-[[(1R,3S)-3-Amino-4-hydroxy-1-phenylbutyl]thio]-6-(difluoromethoxy)-3-pyridinecarbonitrile (E)-butenedioate

a) 5-(4,5-Dihydro-4,4-dimethyl-2-oxazolyl)-3-(methylthio)-2-pyridinnol

To 2,2,6,6-tetramethylpiperidine (5.7 g) in THF, under nitrogen, at 0° C., was added n-BuLi (16.4 ml of 2.5M solution in hexanes) dropwise and stirred for 15 min. The reaction mixture was cooled to −78° C. and 5-(4,5-dihydro-4,4-dimethyl-2-oxazolyl) 2-pyridinol (2.6 g) in THF (75 ml) was added dropwise. The cooling bath was removed and the reaction temperature was allowed to warm to −20° C. and kept at this temperature for 2 h. It was then cooled to −78° C. and dimethyldisulphide (4.9 ml) was added dropwise. There was an exotherm to −30° C. during this addition. The cooling bath was then removed and the reaction was stirred at 20° C. for 12 h. Water (50 ml) was added and the resulting mixture was extracted with dichloromethane (2×60 ml). The combined organics were washed with citric acid (50 ml of 1M aqueous solution), dried (Na₂SO₄) and evaporated to give the sub-title compound as a pale yellow solid, (3.75 g) which was a 50:50 mixture of starting reagent and product by NMR.

MS APCI+ve ^{m}/z 239 [M+H]⁺.

¹H NMR 300 MHz (d₆-DMSO): (product); 7.92 (1H, s), 6.28 (1H, s), 4.06 (3H, s), 2.50 (2H, s), 1.35 (6H, s).

b) 2-(Difluoromethoxy)-5-(4,5-dihydro-4,4-dimethyl-2-oxazolyl)-4-(methylthio)-pyridine

The product from step a) (2.6 g) in NMP (5 ml) was treated with sodium hydride (1.7 g of a 60% dispersion in mineral oil) and heated at 50° C. for 3 h. Chlorodifluoromethane was then bubbled through the reaction mixture for 1 h. Water (50 ml) was added and the resulting mixture was extracted with ethyl acetate (3×60 ml). The combined organics were washed with aqueous NaHCO₃, then brine, dried (MgSO₄) and evaporated to obtain an oil. The residue was purified by chromatography (silica, isohexane/ethyl acetate as eluent) to give sub-title compound (0.6 g) as an oil.

MS APCI+ve ^{m}/z 289 [M+H]⁺.

¹H NMR 400 MHz (d₆-DMSO) 8.50 (1H, s), 7.50 (1H, t), 6.68 (1H, s), 4.06 (2H, s), 2.43 (3H, s), 1.56 (6H, s).

c) 6-(Difluoromethoxy)-4-(methylthio)-3-pyridinecarbonitrile

The sub-title compound was prepared by the method of Example 10 step k) using the product from step b).

¹H NMR 300 MHz (d₆-DMSO) 8.30 (1H, s), 7.49 (1H, t), 6.68 (1H, s), 2.57 (3H, s).

d) 6-(Difluoromethoxy)-4-(methylsulfonyl)-3-pyridinecarbonitrile

The product from step c) (0.36 g) in acetone (15 ml) was treated with NaHCO₃ (1.1 g), then a solution of oxone (3 g) in water (15 ml) was added dropwise and stirred at room temperature for 5 h. Water was added and the resulting mixture was extracted with ethyl acetate (3×50 ml). The combined organics were washed with water, brine and dried (MgSO₄) and then evaporated to give the sub-title compound as a pale yellow solid, (0.25 g).

¹H NMR 300 MHz (d₆-DMSO) 8.75 (1H, s), 7.67 (1H, s), 7.51 (1H, t), 3.38 (3H, s).

e) 4-[[(1R,3S)-3-Amino-4-hydroxy-1-phenylbutyl]thio]-6-(difluoromethoxy)-3-pyridinecarbonitrile (E)-butenedioate

The title compound was prepared by the method of Example 10 steps m) to n) using product from step d). M.p. 144-146° C.

MS APCI+ve ^{m}/z 366 [M+H]⁺.

¹H NMR 400 MHz (d₆-DMSO) 8.61 (1H, s), 7.65 (1H, t), 7.65-7.37 (7H, m), 6.54 (2H, s), 5.34 (1H, m), 3.47 (2H, m), 2.88 (1H, bs), 2.27 (2H, m).

### EXAMPLE 15

### 2-[[(1R,3R)-3-Amino-4-hydroxy-1-phenylbutyl]thio]-6-methyl-3-pyridinecarbonitrile Hydrochloride

a) 1,1-Dimethylethyl (4R) 4-[(2R)-2-[(3-Cyano-6-methyl-2-pyridinyl)thio]-2-phenylethyl]-2,2-dimethyl-3-oxazolidinecarboxylate

The product from Example 11 step b), (190 mg) and 2-chloro-6-methyl-3-pyridinecarbonitrile (220 mg) were dissolved in 7M ammonia in methanol (5 ml) and stirred at ambient temperature for 18 hr. The reaction mixture was evaporated to dryness and the residue was purified by chromatography (silica, dichloromethane as eluent) to give sub-title compound (110 mg) as a gum.

MS APCI+ve ^{m}/z 454 [M+H]⁺.

b) 2-[[(1R,3R)-3-Amino-4-hydroxy-1-phenylbutyl]thio]-6-methyl-3-pyridinecarbonitrile Hydrochloride

A solution of the product from step a) (110 mg) in 4M HCl in dioxane (2 ml) was stirred at 20° C. for 2 hr. The solvent was removed in vacuo and the residue triturated with acetonitrile to give the title compound as a white solid (42 mg).

MS APCI+ve ^{m}/z 314 [M+H]⁺.

¹H NMR 300 MHz (d₆-DMSO) 8.1 (1H, d), 7.54-7.28 (5H, m), 5.36 (1H, t), 5.22-5.17 (1H, m) 3.81-3.75 (1H, m), 3.62-3.54 (1H, m) 3.32 (3H, s), 2.8-2.7 (1H, m), 2.53-2.46 (1H, m).

### EXAMPLE 16

### 4-[[(1R,3S)-3-Amino-4-hydroxy-1-phenylbutyl]thio]-6-(²H₃)methoxy-3-pyridinecarbonitrile (E)-2-butenedioate

a) 5-(4,5-Dihydro-4,4-dimethyl-2-oxazolyl)-2-(²H₃)methoxy-pyridine

Sodium hydride (800 mg) was added to a solution of the product from Example 10 step h) (2.1 g) and methyl-d₃-alcohol-d (720 mg) in DMF (50 ml). The reaction mixture was heated at 65° C. for 2 h and then allowed to cool to room temperature. The mixture was poured into water (1000 ml) and extracted with ethyl acetate (twice). The combined organics were dried (MgSO₄), filtered and concentrated in vacuo to give the sub-title compound (2.3 g) as a colourless oil.

¹H NMR⁴⁰⁰ MHz (CDCl₃) 8.68 (1H, s), 8.10 (1H, d), 6.72 (1H, d), 4.09 (2H, s), 1.37 (6H, s).

b) 5-(4,5-Dihydro-4,4-dimethyl-2-oxazolyl)-2-(²H₃)methoxy-4-(methylthio)-pyridine

The sub-title compound was prepared by the method of Example 10 step j) using the product from step a).

MS APCI+ve ^{m}/z 256 ([M(+H)]⁺).

c) 6-(²H₃)Methoxy-4-(methylthio)-3-pyridinecarbonitrile

The sub-title compound was prepared by the method of Example 10 step k) using the product from step b).

MS APCI+ve ^{m}/z 184 ([M(+H)]⁺).

d) 6-(²H₃)Methoxy-4-(methylsulfonyl)-3-pyridinecarbonitrile

A solution of Oxone (11.1 g) in water (40 ml) was added to a suspension of the product from step c) (1.1 g) in acetone (40 ml) and sodium bicarbonate (4.16 g). The reaction mixture was then stirred at room temperature for 24 h. Water and ethyl acetate were then added until a complete solution was achieved. The organic phase was separated and dried (MgSO₄), filtered and concentrated to give the sub-title compound (1.3 g) as a colourless solid.

MS APCI+ve ^{m}/z 216 ([M(+H)]⁺).

e) 1,1-Dimethylethyl (4S)-4-[(2R)-2-[(2-(²H₃)methoxy-5-methyl-4-pyridinyl)thio]-2-phenylethyl]-2,2-dimethyl-3-oxazolidinecarboxylate

The sub-title compound was prepared by the method of Example 10 step m) using the product from step d).

MS APCI+ve ^{m}/z 373 ([M(+H)-BOC]⁺).

f) 4-[[(1R,3S)-3-Amino-4-hydroxy-1-phenylbutyl]thio]-6-(²H₃)methoxy-3-pyridinecarbonitrile (E)-2-butenedioate

The title compound was prepared by the method of Example 10 step n) using the product from step e). M.p. 181-182° C.

¹H NMR 400 MHz (d₆-DMSO) 8.53 (1H, s), 7.54 (2H, d), 7.38 (1H, t), 7.30 (1H, t), 7.00 (1H, s), 6.45 (1H, s), 5.12 (1H, m), 3.33 (3H, m), 2.64 (1H, m), 1.99 (1H, m), 1.85 (1H, m).

### EXAMPLE 17

### 2-[[(1R,3S)-3-Amino-4-hydroxy-1-phenylbutyl]thio]-6-ethyl-3-pyridinecarbonitrile ethanedioate

a) 2-Chloro-6-ethyl-3-pyridinecarbonitrile

To a stirred solution of 2-chloro-6-methyl-3-pyridinecarbonitrile (1.52 g) in dry DMF (10 ml) under a nitrogen atmosphere, was added iodomethane (2.5 ml). Sodium hydride (60% dispersion, 400 mg) was then added to the stirred solution. After the initial exothermic reaction subsided the mixture was stirred for 0.5 h then diluted with water (50 ml), and the products extracted with diethyl ether (100 ml). The dried extract (MgSO₄) was concentrated to dryness, and the residue was purified by chromatography (silica, isohexane/diethyl ether 4:1) to give the sub-title compound (700 mg).

¹H 400 MHz (CDCl₃) 7.8 (1H, d), 7.16 (1H, d), 2.81 (2H, q), 1.26 (3H, t).

b) 1,1-Dimethylethyl (4S) 4-[(2R)-2-[(3-cyano-6-ethyl-2-pyridinyl)thio]-2-phenylethyl]-2,2-dimethyl-3-oxazolidinecarboxylate

A solution of the product from step a) (200 mg) and product from Example 1 step b) (442 mg) were stirred at ambient temperature in 7M ammonia in methanol (15 ml) for 2 h. The mixture was then concentrated to dryness and the residue purified by chromatography (silica isohexane/diethyl ether 7:3) to afford the sub-title compound (260 mg).

MS APCI+ve ^{m}/z 468 ([M+H]⁺)

c) 2-[[(1R,3S)-3-Amino-4-hydroxy-1-phenylbutyl]thio]-6-ethyl-3-pyridinecarbonitrile ethanedioate

The title compound was prepared from the product from step b), according to the procedure described in Example 8 step c), and was isolated as a colourless solid 80 mg.

¹H 400 MHz (DMSO-d₆) 8.09 (1H, d), 7.5 (2H, d), 7.37-7.19 (4H, m), 5.35 (1H, t), 3.58-3.44 (2H, m), 3.09-3.04 (2H, m), 2.85 (2H, q), 2.35-2.25 (2H, m), 1.26 (3H, t).

MS APCI+ve ^{m}/z 328 ([M+H]⁺).

### EXAMPLE 18

### 2-[[(1R,3S)-3-Amino-4-hydroxy-1-phenylbutyl]thio]-6-(1-methylethyl)-3-pyridinecarbonitrile ethanedioate

a) 2-[[[(1R)-2-[(4S)-2,2-Dimethyl-4-oxazolidinyl]-1-phenylethyl]thio]-6-(1-methylethyl)-3-pyridinecarbonitrile

The sub-title compound was synthesised from 2-chloro-6-(1-methylethyl)-3-pyridinecarbonitrile according to the procedure described in Example 17 step b).

MS APCI+ve ^{m}/z 382 ([M+H]⁺).

b) 2-[[(1R,3S)-3-Amino-4-hydroxy-1-phenylbutyl]thio]-6-(1-methylethyl)-3-pyridinecarbonitrile ethanedioate

The title compound was synthesised from the product from step a) according to the procedure described in Example 8 step c).

MS APCI+ve ^{m}/z 342 ([M+H]⁺).

¹H 300 MHz (DMSO-d₆) 8.1 (1H, d), 7.5-7.19 (6H, m), 5.37 (1H, t), 3.6-3.4 (2H, m), 3.16-3.0 (2H, m), 2.28 (2H, t), 1.27 (3H, d), 1.23 (3H, d).

### EXAMPLE 19

### 2-[[(1R,3S)-3-Amino-4-hydroxy-1-phenylbutyl]thio]-6-methyl]-3-pyridinemethanol ethanedioate

a) Methyl 6-methyl-2-(methylsulfonyl)pyridine-3-carboxylate

A mixture of methyl 2-chloro-6-methylpyridine-3-carboxylate (1 g) and sodium methanesulphinate (1.6 g) in dry DMSO (10 ml) was heated at 120° C. for 4 h. The cooled reaction mixture was diluted with water (100 ml) and the products extracted into ethyl acetate (2×100 ml). The dried extracts (MgSO₄) were concentrated to dryness and the residue purified by chromatography (silica, diethyl ether). The sub-title compound was isolated as a pale pink oil (600 mg).

MS APCI+ve ^{m}/z 230 ([M+H]⁺).

b) Methyl 2-[[(1R)-2-[(4S)-3-[(1,1-dimethylethoxy)carbonyl]-2,2-dimethyl-4-oxazolidinyl]-1-phenylethyl]thio]-6-methyl pyridine-3-carboxylate

The sub-title compound was prepared from the product from step a according to the procedure described in Example 10 step m).

MS APCI+ve ^{m}/z 487 ([M+H]⁺).

c) 1,1-Dimethylethyl (4S)-4-[(2R)-2-[[3-(hydroxymethyl)-6-methyl-2-pyridinyl]thio]-2-phenylethyl]-2,2-dimethyl-3-oxazolidinecarboxylate

A solution of the product from step b) (500 mg) in dry THF at ambient temperature, and under a nitrogen atmosphere was treated with lithiun borohydride (2M solution in THF 5×5 ml aliquots over 3 days). After 5 days the mixture was diluted with water (100 ml) and the excess reagent destroyed by addition of citric acid. The mixture was then extracted with ethyl acetate (2×75 ml) and the combined extracts dried (MgSO₄) and concentrated. The crude product was purified by chromatography (silica diethyl ether/isohexane 7:3) to afford the title compound as a colourless gum (400 mg).

MS APCI+ve ^{m}/z 459 ([M+H]⁺).

d) 2-[[(1R,3S)-3-Amino-4-hydroxy-1-phenylbutyl]thio]-6-methyl-3-pyridinemethanol ethanedioate

The title compound was prepared from the product from step c) according to the procedure described in Example 8 step c).

¹H 300 MHz (DMSO-d₆/D₂O) 7.6-7.2 (6H, m), 6.97 (1H, d), 5.28 (1H, t), 4.36 (2H, s), 3.63-3.38 (2H, m), 3.15 (1H, t), 2.5 (3H, s), 2.31 (2H, t).

MS APCI+ve ^{m}/z 319 ([M+H]⁺).

### EXAMPLE 20

### 6-Acetyl-2-[[(1R,3S)-3-amino-4-hydroxy-1-phenylbutyl]thio]-3-pyridinecarbonitrile Hydrochloride

a) 6-Acetyl-2-(methylsulfonyl)-3-pyridinecarbonitrile

6-Acetyl-2-(methylthio)-3-pyridinecarbonitrile (170 mg) was dissolved in acetone (40 ml) and water (8 ml). Oxone (1.66 g) was added and the suspension stirred at room temperature for 68 h. 0.5M aqueous sodium thiosulphate solution (50 ml) was added and the solution stirred for 0.5 h. The reaction was then extracted with ethyl acetate (3×50 ml) and combined organic extracts washed with water (3×20 ml), dried (MgSO₄) and evaporated in vacuo. The residue was purified by chromatography (silica, hexane/ethyl acetate as eluent) to give the sub-title compound (109 mg) as a white solid.

¹H NMR 300 MHz (CDCl₃) 8.40 (2H, dd), 3.47 (3H, s), 2.78 (3H, s).

b) (4S)-1,1-Dimethylethyl 4-[(2R)-2-[(6-acetyl-3-cyano-2-pyridinyl)thio]-2-phenylethyl]-2,2-dimethyl-3-oxazolidinecarboxylate

The sub-title compound was prepared by the method of Example 10 step m) using the product of step a) (100 mg) and the product of Example 10 step g) (199 mg). The product was purified by chromatography (silica, hexane/ethyl acetate as eluent) to give the sub-title compound (125 mg) as a colourless oil.

¹H NMR 400 MHz (CDCl₃) 7.89 (1H, s), 7.71 (1H, d), 7.46 (2H, t), 7.32 (2H, t), 7.23 (1H, d), 5.16 (1H, m), 4.16 (1H, m), 3.86 (1H, m), 3.51 (1H, m), 2.75-2.62 (3H, d), 2.60-2.33 (1H, m), 2.23-2.10 (1H, m), 1.59-1.40 (15H, m).

c) 6-Acetyl-2-[[(1R,3S)-3-amino-4-hydroxy-1-phenylbutyl]thio]-3-pyridinecarbonitrile Hydrochloride

The product of step b) (125 mg) was dissolved in methanol (20 ml) and the solution treated with 4M HCl in dioxane (10 ml). The reaction was stirred at room temperature for 3 h. The solvent was removed in vacuo and the residue triturated with 20% ethyl acetate in hexane. The solid was filtered and dried to give the title compound (75 mg) as a pale yellow solid. M.p. 78° C.

MS APCI+ve ^{m}/z 342 ([M+H]⁺).

¹H NMR 300 MHz (DMSO-D₆) 8.41 (1H, dd), 8.16 (3H, s), 7.76 (1H, dd), 7.58 (2H, d), 7.39 (2H, t), 7.30 (1H, m), 5.46 (1H, t), 5.35 (1H, t), 3.59-3.40 (2H, m), 3.07 (1H, s), 2.76 (3H, d), 2.34 (2H, t).

### EXAMPLE 21

### 2-[[(1R,3S)-3-Amino-4-hydroxy-1-phenylbutyl]thio]-6-(hydroxymethyl)-3-pyridinecarbonitrile (E)-butenedioate

a) 6-(Hydroxymethyl)-2-(methylthio)-3-pyridinecarbonitrile

6-Formyl-2-(methylthio)nicotinonitrile (500 mg) was dissolved in ethanol (50 ml) and the solution treated with sodium borohydride (117 mg). The reaction was stirred at room temperature for 1 h and then quenched with water (50 ml). The reaction was concentrated in vacuo down to approximately 50 ml and then extracted with ethyl acetate (3×60 ml). Combined organic extracts were washed with water (2×40 ml), dried (MgSO₄) and evaporated in vacuo to give the sub-title compound (478 mg) as a white solid.

¹H NMR 300 MHz (CDCl₃) 7.79 (1H, d), 7.07 (1H, d), 4.80 (2H, d), 3.18 (1H, t), 2.65 (3H, s).

b) 6-[[[(1,1-Dimethylethyl)dimethylsilyl]oxy]methyl]-2-(methylthio)-3-pyridinecarbonitrile

The product from step a) (473 mg) was dissolved in dichloromethane (80 ml) and treated with imidazole (196 mg). The solution was cooled to 0° C. and t-BDMSCl (434 mg) added. The reaction was stirred at room temperature for 18 h and then quenched with water (50 ml). Extracted with ethyl acetate (3×60 ml) and combined organic extracts washed with (2×40 ml), dried (MgSO₄) and evaporated in vacuo to give the sub-title compound (731 mg) as a white solid.

¹H NMR 300 MHz (CDCl₃) 7.79 (1H, d), 7.27 (1H, d), 4.80 (2H, s), 2.59 (3H, s), 0.98 (9H, s), 0.13 (6H, s).

c) 6-[[[(1,1-Dimethylethyl)dimethylsilyl]oxy]methyl]-2-(methylsulfonyl)-3-pyridinecarbonitrile

The product from step b) (725 mg) was dissolved in acetone (80 ml), water (40 ml) and aqueous saturated sodium bicarbonate solution (20 ml). The suspension was treated with oxone (4.1 g) and the reaction stirred at room temperature for 24 h. The reaction mixture was concentrated in vacuo to approximately 70 ml and extracted with ethyl acetate (3×60 ml). Combined organic extracts were washed with water (3×40 ml), dried (MgSO₄) and evaporated in vacuo to give the sub-title compound (715 mg) as a white solid.

¹H NMR 300 MHz (CDCl₃) 8.24 (1H, d), 7.91 (1H, d), 4.92 (2H, s), 3.35 (3H, s), 0.97 (9H, s), 0.16 (6H, s).

d) 1,1-Dimethylethyl (4S)-4-[(2R)-2-[[3-cyano-6-[[[(1,1-dimethylethyl)dimethylsilyl]oxy]methyl]-2-pyridinyl]thio]-2-phenylethyl]-2,2-dimethyl-3-oxazolidinecarboxylate

The sub-title compound was prepared by the method of Example 10 step m) using the product of step c) (222 mg) and the product of Example 10 step g) (300 mg). The product was purified by chromatography (silica, hexane/ethyl acetate as eluent) to give the sub-title compound (180 mg) as a colourless oil.

¹H NMR 300 MHz (CDCl₃) 7.75 (1H, d), 7.39 (2H, d), 7.33-7.18 (4H, m), 5.20-5.00 (1H, m), 4.89-4.67 (2H, m), 4.17-4.04 (1H, m), 3.85 (1H, s), 3.72-3.42 (1H, m), 2.66-2.33 (1H, m), 2.17 (1H, dd), 1.57-1.39 (15H, m), 0.96 (9H, d), 0.14 (6H, d).

e) 2-[[(1R,3S)-3-Amino-4-hydroxy-1-phenylbutyl]thio]-6-(hydroxymethyl)-3-pyridine carbonitrile (E)-butenedioate

The title compound was prepared, by the method of Example 10 step n) using the product of step d) (175 mg), as an off-white solid (100 mg). M.p. 147-149° C.

¹H NMR 300 MHz (d₆-DMSO) 8.17 (1H, d), 7.50 (2H, d), 7.39-7.23 (4H, m), 6.46 (2H, s), 5.33 (1H, t), 4.69 (2H, dd), 3.51-3.34 (2H, m), 2.83 (1H, t), 2.35-2.14 (2H, m).

MS APCI+ve ^{m}/z 330 ([M+H]⁺).

### EXAMPLE 22

### 2-[[(1R,3S)-3-Amino-4-hydroxy-1-phenylbutyl]thio]-3-pyridinecarbonitrile (E)-butenedioate

a) 1,1-Dimethylethyl (4S)-4-[(2R)-2-[(3-cyano-2-pyridinyl)thio]-2-phenylethyl]-2,2-dimethyl-3-oxazolidinecarboxylate

The product from Example 10 step g) (318 mg) was dissolved in 7M ammonia in methanol (40 ml) and 2-chloro-3-cyanopyridine (100 mg) added. The reaction was stirred at room temperature for 24 h. The solvent was removed in vacuo and the residue purified by chromatography (silica, ethyl acetate/hexane as eluent) to give the sub-title compound (200 mg) as a colourless oil.

MS APCI+ve ^{m}/z 440 ([M+H]⁺).

b) 2-[[(1R,3S)-3-Amino-4-hydroxy-1-phenylbutyl]thio]-3-pyridinecarbonitrile (E)-butenedioate

The title compound was prepared, by the method of Example 10 step n) using the product of step a) as an off-white solid (125 mg). M.p. 67-69° C.

¹H NMR 300 MHz d₆-(DMSO) 8.74 (1H, d), 8.21 (1H, dd), 7.50 (2H, d), 7.32 (4H, m), 6.46 (2H, s), 5.37 (1H, t), 3.53-3.33 (2H, m), 2.90-2.80 (1H, m), 2.36-2.17 (2H, m).

MS APCI+ve ^{m}/z 300 ([M+H]⁺).

### EXAMPLE 23

### (β¹S,δ¹R)-β-Amino-δ-[(2,5-dichloro-4-pyridinyl)thiobenzenebutanol] Hydrochloride

a) 2,5-Dichloro-4-(methylthio)-pyridine

To DMF (3.13 ml) in THF (20 ml), under nitrogen, at 0° C., was added nBuLi (8.9 ml of a 2.5M solution in hexanes) dropwise and stirred for 15 min. The reaction mixture was added dropwise to a solution of 2,5-dichloropyridine (3 g) in THF (20 ml) at −78° C. After 2 h, dimethyldisulfide (2.4 ml) was added and the reaction temperature was allowed to warm up to 0° C. Water was added and the mixture was extracted with ethyl acetate. The combined organics were dried (Na₂SO₄) and evaporated to give the sub-title compound as a yellow solid, (3 g).

¹H NMR 400 MHz (CDCl₃) 8.18 (1H, s), 7.02 (1H, s), 2.50 (3H, s).

b) 2,5-Dichloro-4-(methylsulfonyl)-pyridine

The sub-title compound was prepared by the method of Example 5 step b) using the product from Example 23 step a). White solid.

¹H NMR 300 MHz (CDCl₃) 8.39 (1H, s), 7.91 (1H, s), 2.90 (3H, s).

c) (β¹S,δ¹R)-β-Amino-δ-[(2,5-dichloro-4-pyridinyl)thiobenzenebutanol]-Hydrochloride

The title compound was prepared by the method of Example 10 steps m & n) using the products from Example 23 step b) and Example 10 step g). Final purification was by reversed phase HPLC followed by treatment with HCl.

MS (APCI+ve) ^{m}/z 343 [M(+H)]⁺.

¹H 400 MHz (DMSO-d₆) 8.37 (1H, s), 8.08 (3H, bs), 7.58 (3H, m), 7.41 (2H, t), 7.33 (1H, tt), 3.54-3.50 (2H, m), 3.41 (1H, dd), 2.96 (1H, bs), 2.33-2.14 (2H, m).

### EXAMPLE 24

### 2-[[(1R,3S)-3-Amino-4-hydroxy-1-phenylbutyl]thio]-5-fluoro-6-methoxy-3-pyridinecarbonitrile-(E)-butenedioate

a) 2-Chloro-5-fluoro-6-methoxy-3-pyridinecarbonitrile

A solution of 2,6-dichloro-5-fluoro-3-pyridinecarbonitrile (2.33 g) and sodium methoxide (1.9 ml of a 25 wt. % solution in methanol) in DMF was heated at 50° C. for 16 h. Further sodium methoxide (0.57 ml) was added and the heating continued for a further 48 h. Water was added and the mixture was extracted with diethyl ether. The combined organics were washed with water, dried (Na₂SO₄) and evaporated to give the sub-title compound as a white solid (2.08 g).

¹H NMR 300 MHz (CDCl₃) 7.58 (1H, d), 4.11 (3H, s).

b) 2-[[(1R,3S)-3-Amino-4-hydroxy-1-phenylbutyl]thio]-5-fluoro-6-methoxy-3-pyridinecarbonitrile-(E)-butenedioate

The title compound was prepared by the method of Example 10 steps m & n) using the products from Example 24 step a) and Example 10 step g).

MS (APCI+ve) ^{m}/z 348 [M(+H)]⁺.

¹H 400 MHz (DMSO-d₆) 8.20 (1H, d), 7.49 (2H, d), 7.36 (2H, t), 7.28 (1H, m), 5.28 (1H, dd), 4.13 (3H, s), 3.31 (2H, m), 2.67 (1H, m), 2.21 (1H, m), 2.08 (1H, m).

### EXAMPLE 25

### 4-[[(1R,3S)-3-Amino-4-hydroxy-1-phenylbutyl]thio]-6-(dimethylamino)-3-pyridinecarbonitrile (E)-2-butenedioate

a) 5-(4,5-Dihydro-4,4-dimethyl-2-oxazolyl)-N,N-dimethyl-2-pyridinamine

A mixture of the product from Example 10 step h) (2.1 g), 2.0 M dimethylamine/THF (80 ml) and toluene (80 ml) was heated at 120° C. in a sealed tube for 16 h. The mixture was then evaporated to dryness and the residue purified by chromatography (silica, ethyl acetate as eluent) to give the sub-title compound (1.55 g) as a pale orange solid.

¹H NMR 400 MHz (CDCl₃) 8.64 (1H, s), 7.97 (1H, d), 6.48 (1H, d), 4.05 (2H, s), 3.14 (6H, s), 1.36 (6H, s).

b) 5-(4,5-Dihydro-4,4-dimethyl-2-oxazolyl)-N,N-dimethyl-4-(methylthio)-2-pyridinamine

The sub-title compound was prepared by the method of Example 10 step j) using the product from step a) and purified by chromatography (silica, isohexane/ethyl acetate as eluent).

MS APCI+ve ^{m}/z 266 ([M(+H)]⁺).

c) 6-(Dimethylamino)-4-(methylthio)-3-pyridinecarbonitrile

The sub-title compound was prepared by the method of Example 10 step k) using the product from step b).

MS APCI+ve ^{m}/z 194 ([M(+H)]⁺).

d) 6-(Dimethylamino)-4-(methylsulfonyl)-3-pyridinecarbonitrile.

The sub-title compound was prepared by the method of Example 16 step d) using the product from step c).

MS APCI+ve ^{m}/z 226 ([M(+H)]⁺).

e) 1,1-Dimethylethyl (4S)-4-[(2R)-2-[[5-cyano-2-(dimethylamino)-4-pyridinyl]thio]2-phenylethyl]-2,2-dimethyl-3-oxazolidincarboxylate

The sub-title compound was prepared by the method of Example 10 step m) using the product from step d).

MS APCI+ve ^{m}/z 483 ([M(+H)]⁺).

f) 4-[[(1R,3S)-3-Amino-4-hydroxy-1-phenylbutyl]thio]-6-(dimethylamino)-3-pyridinecarbonitrile (E)-2-butenedioate

The title compound was prepared by the method of Example 10 step n) using the product from step e). M.p. 175-177° C.

¹H NMR 400 MHz (d₆-DMSO) 8.29 (1H, s), 7.55 (2H, d), 7.38 (2H, t), 7.29 (1H, t), 6.47 (4H, d), 5.11 (1H, m), 3.38 (2H, m), 3.05 (6H, s), 2.75 (1H, m), 2.17 (1H, m), 2.04 (1H, m).

### EXAMPLE 26

### 4-[[(1R,3S)-3-Amino-4-hydroxy-1-phenylbutyl]thio]-6-(methylamino)-3-pyridinecarbonitrile dihydrochloride

a) 5-(4,5-Dihydro-4,4-dimethyl-2-oxazolyl)-N-methyl-2-pyridinamine

A mixture of the product from Example 10 step h) (2.1 g), 2.0 M methylamine/THF (30 ml) and toluene (30 ml) was heated at 120° C. in a sealed tube for 16 h. The mixture was then evaporated to dryness and the residue purified by chromatography (silica, ethyl acetate as eluent) to give the sub-title compound (700 mg) as a beige solid.

¹H NMR 400 MHz (CDCl₃) 8.60 (1H, s), 7.97 (1H, d), 6.36 (1H, d), 4.85 (1H, br s), 4.06 (2H, s), 2.96 (3H, d), 1.36 (6H, s).

b) 1,1-Dimethylethyl [5-(4,5-dihydro-4,4-dimethyl-2-oxazolyl)-2-pyridinyl]methyl carbamate

Di-tert-butyl dicarbonate (1.47 g) was added to a solution of the product from step a) (700 mg) in dichloromethane (10 ml). 4-(Dimethylamino)pyridine (42 mg) was then added and the mixture was stirred at room temperature for 16 h. The reaction mixture was poured onto water and the organic phase separated, dried (MgSO₄), filtered and evaporated to dryness to give the sub-title compound (900 mg) as colourless oil.

MS APCI+ve ^{m}/z 306 ([M(+H)]⁺).

c) 1,1-Dimethylethyl [5-(4,5-dihydro-4,4-dimethyl-2-oxazolyl)-4-(methylthio)-2-pyridinyl]methyl-carbamate

The sub-title compound was prepared by the method of Example 10 step j) using the product from step b).

MS APCI+ve ^{m}/z 252 ([M(+H)]⁺).

d) 1,1-Dimethylethyl [5-cyano-4-(methylthio)-2-pyridinyl]methyl carbamate

The sub-title compound was prepared by the method of Example 10 step k) using the product from step c).

MS APCI+ve ^{m}/z 180 ([M(+H)]⁺).

e) 1,1-Dimethylethyl [5-cyano-4-(methylsulfonyl)-2-pyridinyl]methyl carbamate

The sub-title compound was prepared by the method of Example 16 step d) using the product from step d).

MS APCI+ve ^{m}/z 212 ([M(+H)]⁺).

f) 1,1-Dimethylethyl (4S) 4-[[[5cyano[˜(1,1-dimethylethoxy)carbonyl]methylaminol-4-pyridinyl]thio]-2-phenylethyl]-2,2-dimethyl-3-oxazolidinecarboxylate

The sub-title compound was prepared by the method of Example 10 step m) using the product from step e).

MS APCI+ve ^{m}/z 569 ([M(+H)]⁺).

g) 4-[[(1R,3S)-3-Amino-4-hydroxy-1-phenylbutyl]thio]-6-(methylamino)-3-pyridinecarbonitrile dihydrochloride

To a solution of the product from step f) (490 mg) in methanol (20 ml), was added 4M HCl in dioxan (20 ml). The mixture was stirred at room temperature for 8 h and the solvent was removed in vacuo. The residue was triturated with diethyl ether and the title compound (340 mg) was collected by filtration as a white solid. M.p. 206-208° C.

MS APCI+ve ^{m}/z 329 ([M(+H)]⁺).

¹H NMR 400 MHz (d₆-DMSO) 8.21 (1H, s), 8.18 (2H, br s), 7.53 (2H, m), 7.36 (2H, m), 7.28 (1H, m), 6.66 (1H, s), 5.04 (1H, t), 3.45 (2H, m), 2.99 (1H, br s), 2.83 (3H, s), 2.31 (2H, t).

### EXAMPLE 27

### (β¹S,β¹R)-β-Amino-δ-[(5-bromo-2-methoxy-4-pyridinyl)thio]-benzenebutanol (E)-2-butenedioate

a) 5-Bromo-2-methoxy-4-(methylthio)-pyridine

To N,N-diisopropylamine (11.7 ml) in THF (45 ml), under nitrogen, at 0° C., was added nBuLi (32.5 ml of a 2.5M solution in hexanes) dropwise and stirred for 15 min. The reaction mixture was cooled to −78° C. and a solution of 5-bromo-2-methoxypyridine (14.3 g) in THF (10 ml) was added dropwise. After 2 h, dimethyldisulfide (13.8 ml) was added followed by THF (20 ml). The reaction temperature was allowed to warm up to −40° C. The reaction was poured into aqueous ammonium chloride solution, and the mixture extracted with ether. The combined organics were dried (Na₂SO₄) and evaporated. Trituration with cold isohexane gave the sub-title compound as a beige solid, (11 g).

¹H NMR 300 MHz (CDCl₃) 8.08 (1H, s), 6.45 (1H, s), 3.91 (3H, s), 2.44 (3H, s).

b) 5-Bromo-2-methoxy-4-(methylsulfonyl)pyridine

The sub-title compound was prepared by the method of Example 16 step d) using the product from step a).

MS APCI+ve ^{m}/z 267/269 ([M(+H)]⁺).

c) 1,1-Dimethylethyl (4S)-4-[(2R)-2-[(5-bromo-2-methoxy-4-pyridinyl)thio]-2-phenylethyl]-2,2-dimethyl-3-oxazolidinecarboxylate

The sub-title compound was prepared by the method of Example 10 step m) using the product from step b).

MS APCI+ve ^{m}/z 523/525 ([M(+H)]⁺).

d) (β¹S,βR)-β-Amino-δ-[(5-bromo-2-methoxy-4-1pyridinyl)thio]-benzenebutanol (E)-2-butenedioate,

The title compound was prepared by the method of Example 10 step n) using the product from step c). M.p. 178-180° C.

MS APCI+ve ^{m}/z 383/385 ([M(+H)]⁺).

¹H NMR 400 MHz (d₆-DMSO) 8.17 (1H, s), 7.56 (2H, d), 7.38 (2H, t), 7.29 (1H, t), 6.86 (1H, s), 6.47 (2H, s), 4.98 (1H, m), 3.79 (3H, s), 3.30-3.41 (2H, m), 2.72 (1H, m), 2.17 (1H, m), 2.04 (1H, m).

### EXAMPLE 28

### (β¹S,β¹R) β-Amino-δ-[(5-chloro-2-methoxy-4-pyridinyl)thio]-benzenebutanol (E)-2-butenedioate

a) 5-Chloro-2-methoxy-4-(methylthio)-pyridine

The product from Example 23 step a) (1.4 g) in methanol (20 ml) was treated with sodium methoxide (8.2 ml of a 25wt % solution in methanol) and heated at reflux for 48 hrs. The solvent was removed under reduced pressure and the residue was partitioned between water (50 ml) and dichloromethane (50 ml). The organic phase was dried (MgSO₄) and evaporated to dryness. Purification by chromatography (silica, dichloromethane as eluent) gave the sub-title compound (345 mg) as a white solid.

MS APCI+ve ^{m}/z 189 ([M(+H)]⁺).

b) 5-Chloro-2-methoxy-4-(methylsulfonyl)pyridine

The sub-title compound was prepared by the method of Example 5 step b) using the product from step a).

MS APCI+ve ^{m}/z 222/224 ([M(+H)]⁺).

c) 1,1-Dimethylethyl (4S)-4-[(2R)-2-[(5-chloro-2-methoxy-4-pyridinyl)thio]-2-phenylethyl]-2,2-dimethyl-3-oxazolidinecarboxylate

The sub-title compound was prepared by the method of Example 10 step m) using the product from step b).

MS APCI+ve ^{m}/z 479/481 ([M(+H)]⁺).

d) (β¹S,β¹R)-β-Amino-δ-[(5-chloro-2-methoxy-4-pyridinyl)thio]-benzenebutanol (E)-2-butenedioate

The title compound was prepared by the method of Example 10 step n) using the product from step c). M.p. 191-193 ° C.

MS APCI+ve ^{m}/z 339-341 ([M(+H)]⁺)

¹H NMR 400 MHz (d₆-DMSO) 8.08 (1H, s), 7.56 (2H, d), 7.38 (2H, t), 7.29 (1H, t), 6.88 (1H, s), 6.48 (2H, s), 4.99 (1H, m), 3.80 (3H, s), 3.30-3.41 (2H, m), 2.73 (1H, m), 2.18 (1H, m), 2.05 (1H, m).

### EXAMPLE 29

### 4-[[(1R,3S)-3-Amino-4-hydroxy-1-phenylbutyl]thio]-6-ethoxy-3-pyridinecarbonitrile. (E)-butenedioate

a) 5-(4,5-Dihydro-4,4-dimethyl-2-oxazolyl)-2-ethoxy-pyridine

The product from Example 10 step h) (2.1 g) in DMF (50 ml) was treated with ethanol (1.2 ml) and sodium hydride (0.8g of a 60% dispersion in mineral oil) and heated at 60° C. for 20 h. Water (200 ml) was added and the resulting mixture was extracted with ethyl acetate (2×150 ml). The combined organics were dried (MgSO₄) and evaporated to give the sub-title compound as a yellow oil, (3.0 g).

MS APCI+ve ^{m}/z 221 [M+H]⁺.

¹H NMR 400 MHz (d₆-DMSO) 8.66 (1H , s), 8.09 (1H, d), 6.71 (1H, d), 4.40 (2H, q), 4.09 (2H, s), 1.26-1.41 (9H, m).

b) 6-Ethoxy-4-(methylthio)-3-pyridinecarbonitrile

The sub-title compound was prepared by the method of Example 10 steps j) to k) from the product from step a).

MS APCI+ve ^{m}/z 195 [M+H]⁺.

¹H NMR 400 MHz (d⁶-DMSO) 8.28 (1H, s), 6.49 (1H, s), 4.42 (2H, q), 2.52 (3H, s), 1.38 (3H, t).

c) 6-Ethoxy-4-(methylsulfonyl)-3-pyridinecarbonitrile

The sub-title compound was prepared by the method from Example 14 step d) from the product from step b).

¹H NMR 400 MHz (d₆-DMSO) 8.67 (1H, s), 7.44 (1H, s), 4.52 (2H, q), 3.27 (3H, s), 1.42 (3H, t).

d) 4-[[(1R,3S)-3-Amino-4-hydroxy-1-phenylbutyl]thio]-6-ethoxy-3-pyridinecarbonitrile (E)-butenedioate

The title compound was prepared by the method of Example 10 steps m) to n) using the product from step c). M.p. 169.5-171.5° C.

MS APCI+ve ^{m}/z 344 [M+H]⁺.

¹H NMR 400 MHz (d₆-DMSO) 8.52 (1H, s), 7.55 (2H, d), 7.39 (2H, t), 7.30 (1H, t), 6.98 (1H, s), 6.47 (2H, s), 5.13 (1H, m), 4.34 (2H, q) 3.40 (2H, m), 2.70 (1H, m), 2.21 (1H, m), 2.02 (1H, m), 1.30 (3H, t).

### EXAMPLE 30

### 3-[[(1R,3S)-3-Amino-4-hydroxy-1-phenylbutyl]thio]-5-(trifluoromethyl)-2-pyridinecarbonitrile ethanedioate

a) 1,1-Dimethylethyl, (4S)-4-[(2R)-2-[[(2-cyano-5-(trifluoromethyl)-3-pyridinyl]thio]-2-phenylethyl]-2,2-dimethyl-3-oxazolidinecarboxylate

The sub-title compound was prepared by the method of Example I step c), using the product from Example 1 step b) and 3-chloro-2-cyano-5-trifluoromethylpyridine.

MS APCI+ve ^{m}/z 408 [M+H-Boc]⁺.

b) 3-[[(1R,3S)-3-Amino-4-hydroxy-1-phenylbutyl]thio]-5-(trifluoromethyl)-2-pyridinecarbonitrile ethanedioate

The title compound was prepared by the method of Example 1 step d), using the product of step a) to give the title compound as a white solid (133 mg). M.p. 147-149° C.

MS APCI+ve ^{m}/z 368 [M+H]⁺.

¹H NMR 400 MHz (d₆-DMSO) 8.98 (1H, s), 8.33 (1H, s), 7.34 (5H, m), 5.04 (1H, t), 3.58 (1H, dd), 3.48 (1H, m), 3.05 (1H, m), 2.33 (1H, m), 2.18 (1H, m).

### EXAMPLE 31

### 3-[[(1R,3S)-3-Amino-4-hydroxy-1-phenylbutyl]thio]-1,6-dihydro-5-methyl-6-oxo-2-pyridinecarbonitrile

a) 3-Bromo-5-methyl-2-pyridinecarbonitrile

A solution of 3-bromo-2-fluoro-5-methylpyridine (J. Het. Chem. 1967, 641, 642) in dry DMSO (100 ml) was treated with sodium cyanide (1.48 g) and heated to 80° C. for 24 h. The mixture was poured into brine, extracted with ethyl acetate and the organic layer dried (MgSO₄). The solvent was evaporated and the residue was purified by chromatography (silica, diethyl ether) to give the sub-title product as a pale yellow solid (3.0 g).

¹H NMR 400 MHz (CDCl₃) 8.47 (1H, s), 7.84 (1H, s), 2.44 (3H, s).

b) 3-Bromo-5-methyl-2-pyridinecarbonitrile-N-oxide

A solution of the product from step a) (2.0 g) in glacial acetic acid (100 ml) was treated with 30% hydrogen peroxide (20 ml) and heated to 80° C. for 22 h. The mixture was evaporated, the residue triturated with hexane and the resulting solid collected to give the sub-title product as a pale yellow solid (2.0 g).

MS APCI+ve ^{m}/z 214 [M+H]⁺.

¹H NMR 400 MHz (CDCl₃) 8.07 (1H, s), 7.35 (1H, s), 2.37 (3H, s).

c) 1,1-Dimethylethyl (4S)-4-[(2R)-2-[(2-cyano-5-methyl-3-pyridinyl)thio]-2-phenylethyl]-2,2-dimethyl-3-oxazolidinecarboxylate N-oxide

The sub-title compound was prepared by the method of Example 1 step c), using the thiobenzoate of Example 1 step b) and the pyridine-N-oxide from step b) (0.43 g) to give a gum (1.25 g), which was used directly in step d).

MS APCI+ve ^{m}/z 470 [M+H]⁺.

d) 1,1-Dimethylethyl (4S)-4-[(2R)-2-[[6-(acetyloxy)-2-cyano-5-methyl-3-pyridinyl]thio]-2-phenylethyl]-2,2-dimethyl-3-oxazolidinecarboxylate

A solution of the product from step d) in acetic anhydride (20 ml) was heated under reflux for 4 h. The mixture was evaporated, the residue was dissolved in ethyl acetate and washed with water, then aqueous NaHCO₃ and dried (MgSO₄). The solvent was evaporated and the residue was purified by chromatography (silica, 20% ethyl acetate/hexane) to give the sub-title product as a viscous oil (0.45 g).

MS APCI+ve ^{m}/z 456 [M+2H-tBu]⁺.

e) 3-[[(1R,3S)-3-Amino-4-hydroxy-1-phenylbutyl]thio]-1,6-dihydro-5-methyl-6-oxo-2-pyridinecarbonitrile

The title compound was prepared by the method of Example 1, step d), using the product of step d) to give the title compound as a white solid (131 mg), isolated as its free base.

MS APCI+ve ^{m}/z 330 [M+H]⁺.

¹H NMR 400 MHz (d₆-DMSO) 7.27 (1H, s), 7.26 (5H, m), 4.53 (1H, m), 3.23 (4H, m), 2.50 (1H, m), 2.12 (1H, m), 1.82 (1H, m), 1.97 (3H, s).

### Example 32

### 3-[[(1R,3S)-3-Amino-4-hydroxy-1-phenylbutyl]thio]-5-chloro-2-pyridinecarbonitrile ethanedioate

The title compound was prepared by the method of Example 10 steps m & n) using 3,5-dichloro-2-pyridinecarbonitrile and the product from Example 10 step g). After treatment with HCl the title compound was purified by reversed phase HPLC (to remove an unwanted regioisomer) and then treated with ethanedioic acid to afford a white solid.

MS (APCI+ve) ^{m}/z 334 [M(+H)]⁺.

¹H 400 MHz (DMSO-d₆) 8.66 (1H, d), 8.22 (1H, d), 8.03 (ca. 2H, vbs), 7.41-7.27 (5H, m), 4.97 (1H, t), 3.55 (1H, dd), 3.44 (1H, dd), 3.02 (1H, m), 2.32 (1H, m), 2.16 (1H, dt).

### EXAMPLE 33

### 6-Amino-4-[[(1R,3S)-3-amino-4-hydroxy-1-phenylbutyl]thio]-3-pyridinecarbonitrile (E)-butenedioate

a) 1,6-Dihydro-4-(methylsulfonyl)-6-oxo-3-pyridinecarbonitrile

6-Methoxy-4-(methylsulfonyl)-3-pyridinecarbonitrile from Example 10 step I) (5.1 g) was dissolved in acetonitrile (200 ml) and sodium iodide (7.28 g) and trimethylsilylchloride (5.28 g) were added. The reaction was heated under reflux for 48 h and then cooled and the solvent evaporated in vacuo. The residue was partitioned between water (120 ml) and ethyl acetate (120 ml). After shaking, the layers were filtered and the solid collected and dried in a vacuum oven at 60° C. to give the sub-title compound as an off-white solid (3.6 g).

¹H NMR 400 MHz (d₆-DMSO) 13.15 (1H, bs), 8.58 (1H, s), 6.89 (1H, s), 3.39 (3H, s).

b) 5-Cyano-4-(methylsulfonyl)-2-pyridinyl trifluoromethanesulfonate

Triflic anhydride. (0.1 ml) was added to a solution of the product from step a) (57 mg) and triethylamine (0.1 ml) in acetonitrile (2 ml) at −20° C. and stirred at −20° C. to 20° C. for 2 h. Water was added and the mixture was extracted with dichloromethane. The organic extracts were dried.(MgSO₄), evaporated and purified by chromatography (silica, dichloromethane as eluent) to give the sub-title compound (66 mg).

¹H 300 MHz (CDCl₃) 8.94 (1H, s), 7.91 (1H, s), 3.37 (3H, s).

c) 6-Amino-4-(methylsulfonyl)-3-pyridinecarbonitrile

0.5M Ammonia in dioxane (2 ml) was added to a solution of the product from step b) (164 mg) in THF (2 ml) and stirred for 16 h. The solvent was removed in vacuo and the residue purified by chromatography (silica, isohexane/ethyl acetate as eluent) to give the sub-title compound (33 mg) as a white solid.

¹H NMR (d6-DMSO) 8.57 (1H, s), 7.78 (2H, s), 7.05 (1H, s), 3.35 (3H, s).

d) 1,1-Dimethylethyl (4S)-4-[(2R)-2-[(2-amino-5-cyano-4-pyridinyl)thio]-2-phenylethyl]-2,2-dimethyl-3-oxazolidinecarboxylate

A solution of the product from Example 10 step g) (105 mg) in 7 M ammonia in methanol (5 ml) was stirred for eight hours. The methanol was evaporated and the residue was dissolved in dry acetonitrile (3 ml). The product from step c) (45 mg) and caesium carbonate (151 mg) were added and the mixture was stirred at 20° C. for 1 h. Ammonium chloride solution was added and the mixture was extracted with ethyl acetate. The organic extracts were dried (MgSO₄), evaporated and purified by chromatography (silica, isohexane/acetone as eluent) gave the sub-title compound (55 mg) as a white solid.

MS (APCI+ve) ^{m}/z 455 [M(+H)]⁺.

¹H 300 MHz (CDCl₃) 8.16 (1H, s), 7.38-7.30 (5H, m), 6.83 (1H, s), 5.22 (2H, s), 4.45 (1H, d), 3.97 (1H, t), 3.55 (1H, t), 2.93 (1H, d), 2.59 (1H, d), 2.29 (1H, q), 1.61-1.42 (15H, m).

e) 6-Amino-4-[[(1R,3S)-3-amino-4-hydroxy-1-phenylbutyl]thio]-3-pyridinecarbonitrile (E)-butenedioate

The title compound was prepared from the product of step d) by the method of Example 10 step n).

MS (APCI+ve) ^{m}/z 315 [M(+H)]⁺.

¹H NMR 400 MHz (DMSO) 8.16 (1H, s), 7.51 (2H, d), 7.38 (2H, t), 7.31 (1H, t), 7.14 (2H, s), 6.62 (1H, s), 6.50 (2H, s), 4.95 (1H, s), 3.41-3.33 (2H, m), 2.78-2.70 (1H, m), 2.29-2.19 (1H, m), 2.16-2.07 (1H, m).

### EXAMPLE 34

### 3-[[(1R,3S)-3-Amino-4-hydroxy-1-phenylbutyl]thio]-5-methyl-2-pyridinecarbonitrile

a) 1,1-Dimethylethyl (4S)-4-[(2R)-2-[(2-cyano-5-methyl-3-Pyridinyl)thio]-2-phenylethyl]-2,2-dimethyl-3-oxazolidinecarboxylate

The sub-title compound was prepared by the method of Example 1 step c), using the thiobenzoate of Example 1 step b) and the bromopyridine from Example 31 step a) (0.17 g) to give the product as a glass (0.19 g).

MS APCI+ve ^{m}/z 398 [M+2H-tBu]⁺.

b) 3-[[(1R,3S)-3-Amino-4-hydroxy-1-phenylbutyl1thio]-5-methyl-2-pyridinecarbonitrile

The title compound was prepared by the method of Example 1 step d), using the product of step a) to give the title compound as a white solid (139 mg), isolated as its free base.

MS APCI+ve ^{m}/z 314 [M+H]⁺.

¹H NMR 400 MHz (d₆-DMSO) 8.41 (1H, s), 8.18 (2H, bs), 8.04 (1H, s), 7.43 (2H, d), 7.31 (3H, m), 5.32 (1H, bt), 5.13 (1H, m), 3.46 (2H, m), 2.93 (1H, m), 2.35 (3H, s), 2.28 (1H, m), 2.16 (1H, m).

### EXAMPLE 35

### 4-[[(1R,3S)-3-Amino-1-(2-fluorophenyl)-4-hydroxybutyl]thio]-6-methoxy-3-pyridinecarbonitrile ethanedioate

a) 1,1-Dimethylethyl 4-[(2S)-2-(2-Fluorophenyl)-2-hydroxyethyl]-2,2-dimethyl-(4S)-3-oxazolidinecarboxylate, and 1,1-Dimethylethyl 4-[(2R)-2-(2-Fluorophenyl)-2-hydroxyethyl]-2,2-dimethyl-(4S)-3-oxazolidinecarboxylate

A stirred suspension of magnesium (243 mg) in THF (30 ml) under nitrogen was treated with 1,2-dibromoethane (2.44 g) and warmed gently. An exotherm set in and the mixture began to reflux. After the metal had dissolved, the mixture was stored at room temperature under nitrogen. A stirred solution of 3-bromofluorobenzene (1.17 g) in THF (10 ml) under nitrogen was treated at −65 to −70° C. with n-butyllithium (2.5 M in hexane, 2.26 ml, 5.67 mmol) and stirred at −70° C. for 30 min. The solution was treated at −65 to −70° C. with the solution of magnesium dibromide from above, stirred at −70 ° C. for 5 min, then at 0° C. for 20 min. A stirred solution of 1,1-dimethylethyl 2,2-dimethyl-4-[(4S)-2-oxoethyl)-3-oxazolidinecarboxylate (1.21 g) in THF (20 ml) under nitrogen was treated at 0° C. with the Grignard reagent formed above, stirred at 0° C. for 30 min, then at room temperature overnight. The solution was quenched with saturated aqueous ammonium chloride and extracted with ether. The washed and dried (MgSO₄) extracts were evaporated and the residue was purified by chromatography (silica, ether/isohexane as eluent) to give the sub-title compound 1,1-dimethylethyl 4-[(2S)-2-(2-Fluorophenyl)-2-hydroxyethyl)-2,2-dimethyl-(4S)-3-oxazolidinecarboxylate as a white solid (600 mg).

¹H NMR 300 MHz (d₄-MeOH) 7.38 (1H, s), 7.28 (4H, s), 7.12 (5H, d), 4.80-4.75 (9H, m), 4.00-3.79 (18H, m), 2.12-1.96 (11H, m), 1.54-1.41 (96H, m).

Further elution gave the second sub-title compound 1,1-Dimethylethyl 4-[(2R)-2-(2-Fluorophenyl)-2-hydroxyethyl]-2,2-dimethyl-(4S)-3-oxazolidinecarboxylate as a white solid (429 mg).

¹H NMR 300 MHz (d₄-MeOH) 7.40-7.37 (1H, m), 7.28 (1H, s), 7.12 (1H, d), 4.83-4.79 (1H, m), 4.06 (1H, s), 3.90-3.84 (1H, m), 3.75-3.72 (1H, m), 2.20 (1H, s), 1.96-1.86 (1H, m), 1.54-1.47 (15H, m).

b) 4-Mercapto-6-methoxy-3-pyridinecarbonitrile.

A mixture of the product from Example 10, step I (1.0 g) and sodium hydrogen sulphide (790 mg) in ethanol (50 ml) was stirred for 2 h and evaporated. The residue was taken up in water, treated with dilute hydrochloric acid until pH 6, and extracted with ethyl acetate. The dried (MgSO4) extracts were evaporated to give the sub-title compound as a tan powder (741 mg).

¹H NMR 300 MHz (CDCl₃) 8.36 (1H, s), 6.72 (1H, s), 3.97 (3H, s).

c) 4-[[3(3S)-Amino-1(1R)-(2-fluorophenyl)-4-hydroxybutyl]thio]-6-methoxy-3-pyridinecarbonitrile ethanedioate

A stirred solution of triphenylphosphine (309 mg) and THF (10 ml) under nitrogen was treated at −5 to 0° C. with DIAD (238 mg), stirred at −5° C. for 20 min, and then cooled to −20° C. and stored. A mixture of the product from step b) (196 mg), and the product from step a) (589 mg) were stirred, treated with the above DIAD/triphenylphosphine solution, stirred overnight and evaporated. The residue was purified by chromatography (silica, ether/isohexane), taken up in methanol (10 ml), treated with 2M HCl in dioxan (10 ml), stirred for 2 h and evaporated. The residue were purified by preparative reversed phase HPLC on a 19×50 mm Xterra C8 5 micron column using 10 to 60% acetonitrile in 2% aqueous 0.880 ammonia solution over 6 min at 20 ml/min. UV detection by DAD. The free base was taken up in ether/ethanol mixture, treated with absolution of oxalic acid in ethanol and evaporated. The residue was triturated with ether and residue was dried to give the title compound as a cream powder (31 mg), M.p. 179-185° C.

MS APCI+ve ^{m}/z 348 [M+H]⁺.

¹H NMR 300 MHz (d₄-MeOH) 8.58 (1H, s), 7.62 (1H, t), 7.43-7.37 (1H, m), 7.31-7.23 (2H, m), 6.98 (1H, s), 5.22 (1H, t), 3.91 (3H, s), 3.56-3.40 (4H, m), 3.05-3.02 (1H, m), 2.40-2.17 (2H, m).

EXAMPLE 36

### 2-[[(1R,3S)-3-Amino-1-(4-fluorophenyl)-4-hydroxybutyl]oxy]-6-trifluoromethyl-3-pyridinecarbonitrile ethanedioate

a) 1,1-Dimethylethyl 4-[(2S)-2-(4-Fluorophenyl)-2-hydroxyethyl]-2,2-dimethyl-(4S)-3-oxazolidinecarboxylate, and 1,1-Dimethylethyl 4-[(2R)-2-(4-Fluorophenyl)-2-hydroxyethyl]-2.2-dimethyl-(4S)-3-oxazolidinecarboxylate

A stirred solution of 1,1-dimethylethyl 2,2-dimethyl-4-[(4S)-2-oxoethyl)-3-oxazolidinecarboxylate

(3.0 g) in THF (20 ml) under nitrogen was treated at 0° C. with 4-flurophenylmagnesium bromide (2M in ether, 8.32 ml) and stirred at 0° C. for 1 h. The solution was quenched with saturated ammonium chloride solution and extracted with ether. The washed and dried (MgSO₄) extracts were evaporated and the residue was is purified by chromatography (silica, ether/isohexane as eluent) to give the sub-title compound 1,1-dimethylethyl 4-[(2S)-2-(4-fluorophenyl)-2-hydroxyethyl]-2,2-dimethyl-(4S)-3-oxazolidinecarboxylate as a white solid (1,62 g).

¹H NMR 300 MHz (d₄-MeOH) 7.40-7.35 (2H, m), 7.10-7.04 (2H, m), 4.72-4.61 (1H, m), 4.02-3.74 (3H, m), 2.05-1.87 (2H, m), 1.55-1.41 (15H, m).

Further elution gave the second sub-title compound 1,1-dimethylethyl 4-[(2R)-2-(2-fluorophenyl)-2-hydroxyethyl]-2,2-dimethyl-(4S)-3-oxazolidinecarboxylate as a white solid (1.21 g).

¹H NMR 300 MHz (d₄-MeOH) 7.39 (2H, m), 7.07 (2H, m), 4.73-4.69 (1H, m), 4.08 (1H, m), 3.92-3.80 (2H, m), 2.15 (1H, m), 1.83 (1H, m), 1.53-1.41 (15H, m).

b) 2-[[(1R,3S)-3-Amino-1-(4-fluorophenyl)-4-hydroxybutyl]oxy]-6-trifluoromethyl-3-pyridinecarbonitrile ethanedioate

A stirred solution of the 2R, 4S isomer from part a) (214 mg) and 2-chloro-6-trifluoromethyl-3-pyridinecarbonitrile (130 mg) in NMP (3 ml) was treated with sodium hydride (60% dispersion in oil, 30 mg), stirred overnight and evaporated. The residue was taken up in ether, washed, dried (MgSO4) and evaporated. The residue was purified by chromatography (silica, ether/isohexane as eluent) to give an oil that was taken up in methanol (5 ml), treated with 4M HCl in dioxane, stirred for 2 h and evaporated. The residues were purified by preparative reversed phase HPLC on a 19×50 mm Xterra C8 5 micron column using 10 to 80% acetonitrile in 2% aqueous 0.880 ammonia solution over 5 min at 20 ml/min. UV detection by DAD. The free base was taken up in ether/ethanol mixture, treated with a solution of oxalic acid in ethanol and evaporated. The residue was triturated with ether and residue was dried to give the title compound as a cream powder (85 mg), M.p. 109-114° C.

MS APCI+ve ^{m}/z 370 [M+H]⁺.

¹H NMR 300 MHz (d₄-MeOH) 8.34 (1H, d), 7.56-7.50 (3H, m), 7.13-7.09 (2H, m), 6.35-6.31 (1H, m), 3.85-3.81 (1H, m), 3.75-3.71 (1H, m), 3.53-3.47 (1H, m), 2.53-2.45 (1H, m), 2.34-2.27 (1H, m).

### EXAMPLE 37

### 2-(2S)-Amino-4-(4R)-(3-fluorophenl)-4-[(4-methoxy-2-nitrophenyl)thio]butan-1-ol

a) 1,1-Dimethylethyl 4-[(2S)-2-(3-fluorophenyl)-2-hydroxyethyl1-2,2-dimethyl-3-(4S)-oxazolidinecarboxylate

The sub-title compound was prepared from 3-fluorophenylmagnesium bromide [from 3-fluorobromobenzene (29.1 g), magnesium (485 mg) and THF (20 ml)] and 1,1-dimethylethyl 2,2-dimethyl-4-[(4S)-2-oxoethyl)-3-oxazolidinecarboxylate (3.0 g) by the method of Example 36, step a) to give a water-white oil (2.06 g).

¹H NMR 300 MHz (d₄-MeOH) 7.39-7.30 (1H, m), 7.18-7.09 (2H, m), 7.02-6.94 (1H, m), 4.75-4.63 (1H, m), 4.02-4.00 (2H, m), 3.76-3.72 (1H, m), 2.02-1.85 (2H, m), 1.55-1.42 (15H, m).

b) 1,1-Dimethylethyl 4-[(2-(benzoylthio)-2(2R)-(3-fluorophenyl)ethyl]-2,2-dimethyl-3(4S)-ozolidinecarboxylate

A stirred solution of triphenylphosphine (8.76 g) in THF (100 ml) under nitrogen was treated dropwise at 0° C. with DIAD (6.75 g), stirred for 30 min, treated with a mixture of thiobenzoic acid (4.61 g) and the alcohol from part a) (5.67 g), stirred overnight and evaporated. The residue was filtered through a pad of silica with dichloromethane/methanol and the filtrate was evaporated. The residue was digested with ether/isohexane and the supernatant was decanted off and evaporated. The residue was purified by chromatography (silica, dichloromethane/isohexane) to give the sub-title compound as a yellow oil (4.8 g) that was used directly for the next stage.

c) 1,1-Dimethylethyl 4-[(2R)-2-(3-Fluorophenyl)-2-mercaptoethyl1-2,2-dimethyl-(4S)-3-oxazolidinecarboxylate

A mixture of the product from step c) (4.8 g) and 7M methanolic ammonia was stirred for 6 h and evaporated to give the sub-title compound as a gum which was taken up in NMP (86 ml) and used directly for the next stage.

MS APCI+ve ^{m}/z 356 [M+H]⁺.

d) 2-(2S)-Amino-4-(3-fluorophenyl)-4-(4R)-](4-methoxy-2-nitrophenyl)thio]butan-1-ol

A mixture of caesium carbonate (717 mg) and 4-chloro-3-nitroanisole (0.2 mmol) was treated with the solution of the thio]from step d) (2 ml) and stirred overnight. The mixture was diluted with water and extracted with methylene chloride. The washed and dried (MgSO₄) extracts were evaporated and the residue was purified by chromatography (silica, ether/isohexane) to give an oil that was taken up in methanol (2 ml), treated with 4M HCl in dioxan (5 ml), stirred for 30 min and evaporated. The residue were purified by preparative reversed phase HPLC on a 19×50 mm Xterra C8 5 micron column using 10 to 60% acetonitrile in 2% aqueous 0.880 ammonia solution over 6 min at 20 ml/min. UV detection by DAD to give the title compound as a yellow oil (5 mg).

MS APCI+ve ^{m}/z 367 [M+H]⁺.

¹H NMR 300 MHz (d₄-MeOH) 7.44-7.38 (2H, m), 7.31-7.24 (1H, m), 7.16-7.05 (3H, m), 6.98-6.91 (1H, m), 4.65-4.60 (1H, m), 3.83 (3H, s), 3.50-3.35 (2H, m), 2.77-2.69 (1H, m), 2.16-2.06 (1H, m), 1.96-1.87 (1H, m).

### EXAMPLE 38

### 2(2S)-Amino-4(4R)-(3-fluorophenyl)-4-[(4-chloro-2-nitrophenyl)thio]butan-1-ol

The title compound was prepared from 1-bromo-4-chloro-2-nitrobezene and the thiol from Example 3, step c) (2 ml) by the method of Example 37 step d) as a yellow oil (14 mg).

MS APCI+ve ^{m}/z 371 [M+H]⁺.

¹H NMR 300 MHz (d₄-MeOH) 8.04-8.03 (1H, m), 7.63 (1H, d), 7.54 (1H, dd), 7.36-7.19 (3H, m), 7.01-6.94 (1H, m), 4.83-4.79 (1H, m), 3.46-3.34 (2H, m), 2.67-2.59 (1H, m), 2.17-2.06 (1H, m), 1.97-1.87 (1H, m).

### EXAMPLE 39

### 2(2S)-Amino-4(4R)-(3-fluorophenyl)-4-[(5-amino-4-chloro-2-nitrophenyl)thio]butan-1-ol

The title compound was prepared from 1-bromo-4-chloro-2-nitrobezene and the thiol from Example 37, step c) (2 ml) by the method of Example 37, step d) as a yellow oil (14 mg).

MS APCI+ve ^{m}/z 386 [M+H]⁺.

¹H NMR 300 MHz (d₄-MeOH) 8.13 (1H, s), 7.37-7.26 (3H, m), 7.03-6.96 (1H, m), 6.83 (1H, s), 4.71-4.66 (1H, m), 3.47-3.34 (2H, m), 2.69-2.61 (1H, m), 2.14-1.90 (2H, m).

### EXAMPLE 40

### 2(2S)-Amino-4(4R)-(3-fluorophenl)-4-[(4-hydroxymethyl)-2-nitrophenyl)thio]butan-1-ol

The title compound was prepared from 1-bromo-4-chloro-2-nitrobezene and the product from Example 37, step c) (2 ml) by the method of Example 37, step d) as a yellow oil (12 mg).

MS APCI+ve ^{m}/z 367 [M+H]⁺.

¹H NMR 300 MHz (d₄-MeOH) 7.97 (1H, d), 7.61 (1H, d), 7.49 (1H, dd), 7.31-7.19 (3H, m), 6.98-6.91 (1H, m), 4.86-4.78 (1H, m), 4.61 (2H, s), 3.46-3.33 (2H, m), 2.68-2.60 (1H, m), 2.13-2.04 (1H, m), 1.97-1.87 (1H, m).

### EXAMPLE 41

### 2(2S)-Amino-4(4R)-(3-fluorophenyl)-4-[(4-fluoro-2-nitrophenyl)thio]butan-1-ol

The title compound was prepared from 1-chloro-4-fluoro-2-nitrobezene and the thiol from Example 37 step c) by the method of Example 37 step d).

MS APCI+ve m/z 355 [M+H]⁺.

¹H NMR 300 MHz (d₄-MeOH) 7.79-7.74 (1H, m), 7.68-7.61 (1H, m), 7.39-7.26 (2H, m), 7.24-7.14 (2H, m), 7.01-6.93 (1H, m), 4.79-4.72 (1H, m), 3.47-3.35 (2H, m), 2.69-2.60 (1H, m), 2.16-2.05 (1H, m), 1.96-1.86 (1H, m).

### EXAMPLE 42

### 2(2S)-Amino-4(4R)-(3-fluorophenyl)-4-[(3.5-dichloro-2-pyridyl)thio]butan-1-ol

The title compound was prepared from 2,3,5-trichloropyridine and the thio]from Example 37 step c) (2 ml) by the method of Example 37 step d) as a water-white oil (25 mg).

MS APCI+ve m/z 361 [M+H]+.

¹H NMR 300 MHz (d₄-MeOH) 8.43 (1H, d), 7.82 (1H, d), 7.37-7.23 (3H, m), 7.02-6.95 (1H, m), 5.28-5.21 (1H, m), 3.48-3.34 (2H, m), 2.71-2.63 (1H, m), 2.26-2.16 (1H, m), 2.08-1.99 (1H, m).

### EXAMPLE 43

### 4-[[(1R,3S)-3-Amino-4-hydroxy-1-phenylbutyl]thio]-3-chlorobenzonitrile ethanedioate

a) 1,1-Dimethylethyl (4S)-[(2R)-2-[(2-chloro-4-cyanophenyll)thio]-2-phenylethyl]-2,2-dimethyl-3-oxazolidinecarboxylate

The sub-title compound (320 mg) was prepared by the method of Example 3 step a) using the product from Example 1 step b) and 3-chloro-4-fluorobenzonitrile.

MS APCI+ve ^{m}/z 3473/5 (M+H⁺)

¹H NMR 400 MHz (d₆-DMSO (90° C.)) 7.87 (1H, d), 7.45-7.62 (4H, m), 7.23-7.34 (3H, m), 4.70 (1H, m), 4.04 (1H,m), 3.78 (1H,m), 3.65 (1H, m), 2.15 (1H, m), 2.06 (1H, m), 1.46 (9H, s), 1.43 (3H,s), 1.39 (3H, s).

b) 4-[[(1R,3S)-3-Amino-4-hydroxy-1-phenylbutyl]thio]-3-chlorobenzonitrile ethanedioate

The title compound (175 mg) was prepared as a white solid (M.p. 142-144° C.) by the method of Example 4 step b) using the product from step a).

MS APCI+ve ^{m}/z 333/5 (M+H⁺)

¹H NMR 400 MHz (d₆-DMSO) 8.02 (1H, s), 7.75 (1H, d), 7.61 (1H, d), 7.52 (2H, m), 7.25-7.4 (3H, m), 5.00 (1H, m), 3.50 (1H, m), 3.39 (1H, m), 2.96 (1H, t), 2.10-2.30 (2H, m).

### EXAMPLE 44

### 4-Chloro-2-[[(1R,3S)-3-(ethylamino)-4-hydroxy-1-(2-thiazolyl)butyl]oxy]-5-fluoro-benzonitrile ethanedioate salt

To a solution of the product from Example 8 step c) (140 mg) in ethanol (4 ml) was added acetaldehyde (35 μl) and the reaction stirred for 16 h. After cooling to 0° C., sodium borohydride (77 mg) was added and the reaction stirred for 30 min. Water (0.5 ml) was added and the mixture was diluted with ethyl acetate and filtered. The solution was dried (MgSO₄) and evaporated. Purification by reversed phase HPLC, neutralisation of relevant fractions and addition of ethanedioic acid (1 eq) gave the title compound. Recrystallisation from ethyl acetate/diethyl ether gave a white solid. M.p. 55-80° C.

MS (APCI+ve) ^{m}/z 370 [M(+H)]⁺.

¹H 400 MHz (CD₃OD) 7.87 (1H, d), 7.70 (2H, m), 7.40 (1H, d), 6.05 (1H, dd), 3.92 (1H, dd), 3.80 (1H, dd), 3.51 (1H, m), 3.16 (2H, m), 2.54 (2H, m), 1.33 (3H, t).

### EXAMPLE 45

### 2-[[(1R,3S)-3-Amino-4-hydroxy-1-(5-thiazolyl)butyl]oxy]-5-fluoro-benzonitrile (2E)-2-butenedioate

a) 1,1-Dimethylethyl (4S)-4-[(2R)-2-(2-chloro-5-thiazolyl)-2-hydroxyethyl]-2,2-dimethyl-3-oxazolidinecarboxylate and 1,1-dimethylethyl (4S)-4-[(2S)-2-(2-chloro-5-thiazolyl)-2-hydroxyethyl]-2,2-dimethyl-3-oxazolidinecarboxylate

Butyl lithium (1,6 M in hexanes, 4.26 ml) was added dropwise to a solution of diisopropylamine (1.59 ml) in THF (20 ml) at −78° C. under a nitrogen atmosphere. After 15 minutes at −78° C. a solution of 2-chlorothiazole (900 mg) in THF (10 ml) was added dropwise and the reaction mixture was stirred cold for 15 minutes. A solution of 1,1-dimethylethyl (4S)-2,2-dimethyl-4-(2-oxoethyl)-3-oxazolidinecarboxylate (1.82 g) in THF (10 ml) was then added over 5 minutes. After the addition was complete the cooling was removed and the mixture was stirred for 30 minutes. The reaction mixture was poured into water and the products extracted with diethyl ether. The combined extracts were dried (MgSO₄), filtered and evaporated under vacuo. Purification by chromatography (silica, 50% isohexane/diethyl ether as eluent) gave the (4S, 2S) sub-title compound (500 mg) as a colourless oil.

¹H NMR 400 MHz (CDCl₃) 7.34 (1H, s), 5.47 (1H, d), 4.80 (1H, d), 4.32 (1H, m), 4.03 (1H, m), 3.73 (1H, d), 2.09 (1H, m), 1.89 (1H, m), 1.53 (15H, m).

Further elution gave the (4S, 2R) sub-title compound (380 mg) as a colourless oil.

¹H NMR 400 MHz (CDCl₃) 7.37 (1H, s), 5.01 (1H, m), 4.73 (1H, br s), 4.18 (1H, br s), 4.05 (1H, m), 3.73 (1H, br d), 2.18 (2H, br d), 1.48 (15H, m).

b) 1,1-Dimethylethyl (4S)-4-[(2R)-2-hydroxy-2-(5-thiazolyl)ethyl]-2,2-dimethyl-3-oxazolidinecarboxylate

10% Palladium on charcoal was added to a solution of the product from step a) (380 mg) and sodium acetate (129 mg) in ethanol (15 ml). The reaction mixture was stirred under 5 atmospheres of hydrogen for 16 h. The mixture was filtered and evaporated. The residue was then dissolved in dichloromethane, re-filtered and evaporated to give the sub-title compound (235 mg) as a colourless oil.

¹H NMR 400 MHz (CDCl₃) 8.73 (1H, br s), 7.76 (1H, s), 5.12 (1H, m), 4.22 (1H, m), 4.04 (1H, m), 3.82 (1H, m), 2.22 (2H, m), 1.48 (15H, s).

c) 1,1-Dimethylethyl (4S) 4-[(2R)-2-(2-cyano-4-fluorophenoxy)-2-(5-thiazolyl)ethyl]-2,2-dimethyl-3-oxazolidinecarboxylate

Caesium carbonate (466 mg) was added to a solution of the product from step b) (235 mg) and 2,5-difluorobenzonitrile (100 mg) in DMF (15 ml). The reaction mixture was then stirred at room temperature for 3 days. The reaction temperature was then increased to 55-60° C. for 5 days. After cooling to room temperature the mixture was diluted with water and extracted with ethyl acetate. The organic phase was dried (MgSO₄), filtered and concentrated in vacuo and the residue was purified by chromatography (silica, isohexane/ethyl acetate as eluent). The sub-title compound (150 mg) was obtained as a colourless oil.

MS APCI+ve ^{m}/z 448 ([M(+H)]⁺).

d) 2-[[(1R3S)-3-Amino-4-hydroxy-1-(5-thiazolyl)butyl]oxy]-5-fluoro-benzonitrile (2E)-2-butenedioate

The title compound was prepared by the method of Example 10 step n) using the product from step c). M.p. 163-165° C.

MS APCI+ve ^{m}/z 308 ([M(+H)]⁺).

¹H NMR 400 MHz (d₆-DMSO) 9.11 (1H, s), 8.04 (1H, s), 7.73 (1H, m), 7.52 (1H, m), 7.41 (1H, m), 6.47 (2H, s), 6.24 (1H, t), 3.55 (1H, m), 3.46 (1H, m), 3.00 (1H, t), 2.30 (1H, m), 2.17 (1H, m).

### EXAMPLE 46

### 2-[[(1R,3S)-3-Amino-4-methoxy-1-phenylbutyl]thio]-6-methyl-3-pyridinecarbonitrile ethanedioate

a) 6-Methyl-2-[[(1R)-1-phenyl-3-butenyl]thio]-3-pyridinecarbonitrile

A mixture of 2-mercapto-6-methyl-3-pyridinecarbonitrile (6.08 g), α-(2-propenyl)-(α¹S)-benzenemethanol (6 g) and triphenylphosphine (13.8 g) was stirred in dry THF (150 ml) at 0° C. To the mixture was added diisopropyl azodicarboxylate (10.4 ml) dropwise over 20 min. The mixture was then allowed to reach ambient temperature and stirred for 17 h. The reaction mixture was then concentrated to dryness and the residue purified by chromatography (silica isohexane/ethyl acetate 95:5) to afford the sub-title compound as a pale yellow oil (9.58 g).

MS APCI+ve ^{m}/z 281 ([M+H]⁺).

b) 2-[[(1R,3R)-3,4-Dihydroxy-1-phenylbutyl]thio]-6-methyl-3-pyridinecarbonitrile

AD-mix β (47.89 g) was added to a vigorously stirred mixture of 2-methyl-2-propanol and water (160 ml of each). The mixture was cooled to 0° C. and the product from step a) (9.58 g) added dropwise to the mixture as a solution in 2-methyl-2-propanol (20 ml). After 20 h at 0° C. the mixture was extracted with ethyl acetate (3×100 ml) and the organic extracts combined, dried (Na₂SO₄) and concentrated to dryness. The mixture was purified by chromatography (silica dichloromethane/7M ammonia in methanol 99:1 to 98:2) to give the sub-title compound (5.39 g).

MS APCI+ve ^{m}/z 315 ([M+H]⁺).

c) 2-[[(1R,3R)-4-[[(1,1-Dimethylethyl)dimethylsilyl]oxy]-3-hydroxy-1-phenylbutyl]thio]-6-methyl-3-pyridinecarbonitrile

Chloro-(1,1-dimethylethyl)dimethylsilane (1.54 g) was added to a stirred mixture of the product from step b) (3.2 g) and imidazole (700 mg) in dry THF (75 ml) at 0° C. The mixture was stirred at 0° C. for 1 h and at 20° C. for 1 h. Extra chloro-(1,1-dimethylethyl)dimethylsilane (750 mg) and imidazole (350 mg) was added and stirring continued for a further 3 h. The mixture was concentrated to dryness and the residue dissolved in diethyl ether (100 ml) and the solution passed through a pad of silica gel. The ethereal solution was then concentrated to dryness to afford the sub-title (3 g).

MS APCI+ve ^{m}/z 429 ([M+H]⁺).

d) 2-[[(1R,3R)-4-[[(1,1-Dimethylethyl)dimethylsilyl]oxy]-3-[(methylsulfonyl)oxy]-1-phenylbutyl]thio]-6-methyl-3-pyridinecarbonitrile

A solution of the product from step c) (5 g) in dry THF (50 ml) at 0° C. was treated with diisopropylethylamine (2.1 ml) and methanesulphonyl chloride (0.91 ml) and the mixture stirred for 1 h. A further 2 equivalents of diisopropylethylamine and methanesulphonyl chloride were added over the next 3 h to complete the reaction. The solvent was then removed under reduced pressure and the residue dissolved in a mixture of dichloromethane and diethyl ether (200 ml 1:1) and the solution passed through a pad of silica gel. The filtrate was collected and combined with further ether washes of the silica gel. Concentration gave the sub-title compound which was used immediately.

MS APCI+ve ^{m}/z 507 ([M+H]⁺).

e) 2-[[(1R,3S)-3-Azido-4-[[(1,1-dimethylethyl)dimethylsilyl]oxy]-1-phenylbutyl]thio]-6-methyl-3-pyridinecarbonitrile

The product from step d) was dissolved in dry DMF (50 ml) and the solution treated with sodium azide (1.52 g). The mixture was heated to 90° C. for 4 h then cooled and diluted with water (100 ml). The products were extracted into diethyl ether (2×100 ml) and the combined extracts dried (MgSO₄) and concentrated to an oil. The crude product was purified by chromatography (silica diethyl ether/isohexane 1:4) to give the sub-title compound (4.9 g).

¹H NMR 400 MHz (CDCl₃) 7.59 (1H, d), 1.43-7.2 (5H, m), 6.86 (1H, d), 5.29 (1H, dd), 3.65-3.54 (2H, m), 3.04 (1H, m), 2.56 (3H, s) 2.25-2.07 (2H, m), 0.83 (9h, s), 0.00(6H, s).

f) 2-[[(1R,3S)-3-Azido-4-hydroxy-1-phenylbutyl]thio]-6-methyl-3-pyridinecarbonitrile

The product from step e) in dry THF (50 ml) containing tetrabutylammonium fluoride (11 ml, 1 molar solution in THF) was stirred at ambient temperature for 20 h. The mixture was concentrated to dryness and the residue dissolved in a mixture of diethyl ether and dichloromethane then passed through a pad of silica gel. The filtrate was concentrated to give the sub-title compound (2.6 g).

MS APCI+ve ^{m}/z 454 ([M+H]⁺).

g) 2-[[(1R,3S)-3-Azido-4-[(methylsulfonyl)oxy]-1-phenylbutyl]thio]-6-methyl-3-pyridinecarbonitrile

An ice cold solution of the product from step f) (0.5 g) and diisopropylethylamine (0.26 ml) in dry THF(20 ml) was treated with methanesulphonyl chloride (0.12 ml). After the addition was complete the mixture was allowed to reach room temperature and stirred for 1 h. More diisopropylethylamine (0.26 ml) and methanesulphonyl chloride (0.12 ml) were added and stirring continued for a further 2 h. The mixture was diluted with water (100 ml) and the products extracted into ethyl acetate (2×50 ml). The combined organic extracts were dried (MgSO₄) and concentrated to an oil. The crude product was purified by chromatography(silica, diethyl ether/isohexane 1:1). The sub-title compound was isolated as an oil (630 mg).

MS APCI+ve ^{m}/z 418 ([M+H]⁺).

h) 2-[(3-Azido-4-methoxy-1-phenylbutyl)thio]-6-methyl-3-pyridinecarbonitrile

A solution of the sulfonate ester from step g) (0.9 g) in methanol (50 ml) was treated with sodium methoxide (1 ml 25 wt/v solution in methanol) and the mixture refluxed for 20 h. The mixture was then concentrated to low volume and treated with 10% aqueous citric acid (20 ml). The products were extracted into diethyl ether (100 ml) and the extract dried (MgSO₄) and concentrated. The crude oil was purified by chromatography (silica, diethyl ether/isohexane 1:4) to afford the sub-title compound as an amber oil (200 mg).

MS APCI+ve ^{m}/z 354 ([M+H]⁺).

i) 2-[[(1R,3S)-3-Amino-4-methoxy-1-phenylbutyl]thio]-6-methyl-3-pyridinecarbonitrile

A solution of azide 46 g (198 mg) and triphenylphosphine in wet THF (10 ml+0.2 ml water) was stirred and heated under reflux for 3 h. The mixture was then concentrated, and the residue purified by chromatography (silica, dichloromethane/7M ammonia in methanol 95:5) to afford the free base (180 mg). The ethanedioic acid salt was prepared by addition of 1 equivalent of ethanedioic acid in acetonitrile to the free base affording a cream coloured solid (180 mg).

MS APCI+ve ^{m}/z 328 ([M+H]⁺).

¹H 400 MHz (d₆-DMSO) 8.08 (1H, d), 7.51-7.19 6H, m), 5.31 (1H, t), 3.47-3.35 (2H, m), 3.21-3.17 (4H, m), 2.6 (3H, s), 2.33 (2H, t).

### EXAMPLE 47

### 2-[[(1R,3S)-3-Amino-4-hydroxy-4-methyl-1-phenylpentyl]oxy]-4-chloro-5-fluoro benzonitrile ethanedioate

a) 1,1-Dimethylethyl (4S) 4-[(2R)-2-hydroxy-2-phenylethyl]-2,2,5,5-tetramethyl-3-oxazolidinecarboxylate

A solution of 1,1-dimethylethyl (4S)-2,2,5,5-tetramethyl-4-(2-oxoethyl)-3-oxazolidinecarboxylate (4.6 g) in dry THF (50 ml) and under an atmosphere of nitrogen was treated at 0° C. with phenylmagnesium bromide (1 molar solution in THF 22 ml). After the addition was complete the reaction was allowed to warm to 20° C., and stirred for 0.5 h. The reaction mixture was quenched with aqueous citric acid (150 ml, 10% w/v), and the products extracted into ethyl acetate (2×75 ml). The combined organic extracts were dried (MgSO₄) and concentrated to a gum. The mixture of diastereomers was separated by chromatography (silica, isohexane/diethyl ether). The title compound was isolated as a colourless solid (1.3 g).

¹H 400 MHz (d₆-DMSO) 7.35-7.20 (5H, m), 5.19 (1H, d), 4.63-4.59 (1H, m), 3.93 (1H, m), 1.9-1.7 (2H, m), 1.50 (3H, s), 1.44 (9H, s), 1.29 (3H, s), 1.26 (3H, s), 1.24 (3H, s).

b) 1,1-Dimethylethyl (4S) 4-[(2R)-2-(5-chloro-2-cyano-4-fluorophenoxy)-2-phenylethyl]-2,2,5,5-tetramethyl-3-oxazolidinecarboxylate

The sub-title compound was prepared according to the procedure described in Example 8 step b), using the product of step a).

MS APCI+ve ^{m}/z 403 ([M+H-boc]⁺).

c) 2-[[(1R,3S)-3-Amino-4-hydroxy-4-methyl-1-phenylpentyl]oxy]-4-chloro-5-fluorobenzonitrile ethanedioate

The title compound was prepared from the compound from step b) by the method of Example 8 step c). M.p 80° C.

¹H 400 MHz (d₆-DMSO) 7.62 (1H, d), 7.49-7.34 (5H, m), 7.17 (1H, d), 5.67 (1H, dd), 3.24 (1H, dd), 2.38-2.25 (2H, m), 1.26 (3H, s), 1.21 (3H, s).

MS APCI+ve ^{m}/z 363 ([M+H)]⁺).

### EXAMPLE 48

### 2-[[(1S,3S)-3-Amino-4-hydroxy-1-propylbutyl]oxy]-4-chloro-5-fluorobenzonitrile ethanedioate

a) 1,1-Dimethylethyl (4s) 4-[(2S)-2-hydroxypentyl]-2,2-dimethyl-3-oxazolidinecarboxylate

The sub-title compound was prepared by the method of Example 47 step a), but using propylmagnesium chloride and 1,1-dimethylethyl (4S)-2,2-dimethyl-4-(2-oxoethyl)-3-oxazolidinecarboxylate.

MS APCI+ve ^{m}/z 188 ([M+H-boc]⁺).

b) 1,1-Dimethylethyl (4S) 4-[(2S)-2-(5-chloro-2-cyano-4-fluorophenoxy)pentyl]-2,2-dimethyl-3-oxazolidinecarboxylate.

The sub-title compound was prepared by the method of Example 8 step b), using the product of step a) in dry THF.

MS APCI+ve ^{m}/z 341 ([M+H-boc]⁺).

c) 2-[[(1S,3S)-3-Amino-4-hydroxy-1-propylbutyl]oxy]-4-chloro-5-fluorobenzonitrile ethanedioate

The title compound was prepared according to the procedure described for the product from Example 8 step c). M.p. 171-2° C.

MS APCI+ve ^{m}/z 301 ([M+H]⁺).

¹H 300 MHz (d₆-DMSO) 8.02 (1H, d), 7.66 (1H, d), 4.79 (1H, m), 3.67-3.61 (1H, m), 3.48-3.42 (1H, m), 3.2 (1H, m), 1.92 (2H, t), 1,66-1.56 (2H, m), 1.5-1.2 (2H, m), 0.89 (3H, t).

### EXAMPLE 49

### 2-[[(1S)-1-[(2S)-2-Amino-3-hydroxypropyl]pentyl]thio]-6-methyl-3-pyridinecarbonitrile ethanedioate

a) 1,1-Dimethylethyl (4 g)-4-[(2R)-2-hydroxyhexyl]-2,2-dimethyl-3-oxazolidinecarboxylate

The sub-title compound was prepared in a similar procedure to that described for the compound from Example 47 step a), but using butylmagnesium chloride and 1,1-dimethylethyl (4S)-2,2-dimethyl-4-(2-oxoethyl)-3-oxazolidinecarboxylate.

¹H 300 MHz (d₆-DMSO) 4.53(1H, d), 4.284.22 (1H, m), 4.00 (1H, dd), 3.66 (1H, d), 3.55-3.42 (1H, m), 1.8-1.71 (1H, m), 1.5-1.3 (21H, m), 0.90 (3H, t).

b) 1,1-Dimethylethyl (4S)-4-[(2s)-2-(benzoylthio)hexyl]-2,2-dimethyl-3-oxazolidinecarboxylate

The sub-title compound was prepared by the method of Example 1 step b), but using the product from Example 49 step a).

MS APCI+ve ^{m}/z 322 ([M+H-boc]⁺).

c) 1,1-Dimethylethyl (4S) 4-[(2S)-2-[(3-cyano-6-methyl-2-pyridinyl)thio]hexyl]-2,2-dimethyl-3-oxazolidinecarboxylate

The sub-title compound was prepared by the method of Example 17 step b), but using the product from Example 49 step b).

MS APCI+ve ^{m}/z 434 ([M+H]⁺).

d) 2-[[(1S)-1-[(2S)-2-Amino-3-hydroxypropyl]pentyl]thio]-6-methyl-3-pyridinecarbonitrile ethanedioate

The title compound was prepared by the method of Example 8 step c) using the product of step c).

MS APCI+ve ^{m}/z 294 ([M+H]⁺).

¹H 400 MHz (d₆-DMSO) 8.09 (1H, d), 7.2 (1H, d), 4.22 (1H, br s), 3.5-3.8 (2H, m), 3.2 (1H, br s), 2.52 (3H, s), 1.5-2.2 (4H, m), 0.93 (4H,d), 0.88 (3H, t).

### EXAMPLE 50

### 2-[[(1S,3S)-3-Amino-4-hydroxy-1-(2-methylpropyl)butyl]thio]-6-methyl-3-pyridinecarbonitrile ethanedioate

a) 1,1-Dimethylethyl (4S)-4-[(2R)-2-hydroxy-4-methylpentyl]-2,2-dimethyl-3-oxazolidinecarboxylate

The sub-title compound was prepared by the method of Example 47 step a) but using isobutylmagnesium chloride and 1,1-dimethylethyl (4S)-2,2-dimethyl-4-(2-oxoethyl)-3-oxazolidinecarboxylate.

MS APCI+ve ^{m}/z 202 ([M+H-boc]⁺).

b) 1,1-Dimethylethyl (4S)-4-[(2S)-2-(benzoylthio)-4-methylpenyl]-2,2-dimethyl-3-oxazolidinecarboxylate

The sub-title compound was prepared by the method of Example 1 step b), but using the product from step a).

MS APCI+ve ^{m}/z 322 ([M+H-boc]⁺).

c) 1,1-Dimethylethyl (4S)-4-[(2S)-2-[(3-cyano-6-methyl-2-pyridinyl)thio]-4-methylpentyl]-2,2-dimethyl-3-oxazolidinecarboxylate

The sub-title compound was prepared by the method of Example 17 step b), but using the product from step b).

MS APCI+ve ^{m}/z 434 ([M+H]⁺).

d) 2-[[(1S,3S)-3-Amino-4-hydroxy-1-(2-methylpropyl)butyl]thio]-6-methyl-3-pyridinecarbonitrile ethanedioate

The title compound was prepared by the method of Example 8 step c), but using the product from step c).

MS APCI+ve ^{m}/z 322 ([M+H]⁺).

¹H 400 MHz (DMSO-d₆) 8.1 (1H, d), 7.2 (1H, d), 4.2-4.1 (1H, m), 3.7-3.5 (2H, m), 3.2 (1H, m), 2.52 (3H, s), 2.1-2 (1H, m), 1.73-1.7 (2H, m), 1.45-1.24 (4H, m), 0.86 (3H, t).

### EXAMPLE 51

### 2-[[(3S)-3-Amino-4-hydroxy-1-(5-isoxazolyl)butyl]thio]-6-methyl-3-pyridinecarbonitrile (E)-butenedioate

a) (4S)-4-[2-(5-Isoxazolyl)-2-oxoethyl]-2-oxazolidinone

The sub-title compound was prepared by the method of Example 2 step a) using 5-isoxazolecarbonyl chloride.

¹H NMR (d6-DMSO) δ 8.84 (1H, d), 7.72 (1H, d), 4.49 (1H, t), 4.37 (1H, t), 4.24 (1H, quintet), 4.06 (1H, dd), 3.92-3.74 (2H, t).

b) (4S)-4-[2-Hydroxy-2-(5-isoxazolyl)ethyl]-2-oxazolidinone

The sub-title compound was prepared by the method of Example 2 step b) using the product of step a).

¹H NMR (d6-DMSO) 8.49 (1H, t), 7.83 & 7.65 (1H, s), 6.37 (1H, dd), 5.90 (1H, dd), 4.87 (1H, dd), 4.43-4.31 (1H, m), 4.10-3.72 (2H, m), 1.99-1.85 (2H, m).

c) (4S)-4-[2-(Benzoylthio)-2-(5-isoxazolyl)ethyl]-2-oxazolidinone

The sub-title compound was prepared by the method of Example 2 step c) using the product of step b).

MS APCI+ve ^{m}/z 318 [M+H]⁺.

d) 2-[[1-(5-Isoxazolyl)-2-[(4S)-2-oxooxazolidinyl]ethyl]thio]-6-methyl-3-pyridinecarbonitrile

The sub-title compound was prepared by the method of Example 2 step d) using the product of step c).

MS APCI+ve ^{m}/z 330 [M+H]⁺.

e) 1,1-Dimethylethyl (4S)-4-[2-[(3-cyano-6-methyl-2-pyridinyl)thio]-2-(5-isoxazolyl)ethyl]-2-oxo-3-oxazolidinecarboxylate

The sub-title compound was prepared by the method of Example 2 step e) using the product of step d).

¹H NMR (CDCl₃) 8.22 (1H, ddd), 7.75 (1H, dd), 7.03 (1H, dd), 6.44 & 6.29 (1H, 2×dd), 5.59 & 5.48-5.40 (1H, t & m), 4.56-4.22 (3H, m), 2.65-2.54 (5H, m), 1,62-1.48 (9H, m).

f) 1,1-Dimethylethyl [(1S)-3-[(3-cyano-6-methyl-2-pyridinyl)thio]-1-(hydroxymethyl)-3-(5-isoxazolyl)propyl]carbamate

The sub-title compound was prepared by the method of Example 2 step f) using the product of step e).

MS APCI+ve ^{m}/z 405 [M+H]⁺.

g) 2-[[(3S)-3-Amino-4-hydroxy-1-(5-isoxazolyl)butyl]thio]-6-methyl-3-pyridinecarbonitrile, (E)-butenedioate

A solution of the product from step f) (48 mg) in 4M HCl in dioxane (2 ml) was stirred for 2 h. 2M Potassium carbonate solution was added and the mixture was as extracted with ethyl acetate. The organic extracts were dried (Na₂SO₄), evaporated and purified by chromatography (silica dichloromethane/7M ammonia in methanol as eluent) then converted into the (E)-butenedioate salt by addition of one equivalent of fumaric acid to give the title compound (1 7 mg) as a white solid. M.p. 150-2° C.

MS APCI+ve ^{m}/z 305 [M+H]⁺.

¹H NMR (d6-DMSO) 8.51 (1H, d), 8.13 (1H, d), 7.24 (1H, dd), 6.54 (1H, dd), 6.43 (2H, s), 5.69 & 5.62 (1H, 2×t), 3.57-3.32 (3H, m), 2.97-2.75 (1H, m), 2.60 (3H, s), 2.43-2.01 (2H, m).

### EXAMPLE 52

### 2-[[(3S)-3-Amino-4-hydroxy-1-(5-isoxazolyl)butyl]oxy]-6-(trifluoromethyl)-3-pyridinecarbonitrile, (E)-butenedioate

a) 2-[1-(5-Isoxazolyl)-2-[(4S)-2-oxo-4-oxazolidinyl]ethoxy]-6-(trifluoromethyl)-3-pyridinecarbonitrile

Caesium carbonate (1.35 g) was added to a solution of the product of Example 51 step b) (330 mg) and 3-chloro-5-(trifluoromethyl)-2-pyridinecarbonitrile (556 mg) in DMF (2 ml) and the mixture was stirred at 20° C. for 1 h. Ammonium chloride solution was added and the mixture was extracted with ethyl acetate. The organic extracts were dried (MgSO₄), evaporated and purified by chromatography (silica, isohexane/ethyl acetate as eluent) gave the sub-title compound (258 mg).

¹H NMR (CDCl₃) 8.25 (1H, d), 8.15 (1H, t), 7.47 (1H, t), 6.53 (1H, t), 6.42 (1H, d), 5.78 &, 5.64 (1H, 2×s), 4.66-4.53 (1H, m),4.29-4.07 (2H, m), 2.68-2.37 (2H, m).

b) 1,1-Dimethylethyl (4S)-4-[2-[[3-cyano-6-(trifluoromethyl)-2-pyridinyl]oxy]-2-(5-isoxazolyl)ethyl]-2-oxo-3-oxazolidinecarboxylate

The sub-title compound was prepared by the method of Example 2 step e) using the product of step a).

¹H NMR (CDCl₃) 8.24 (1H, d), 8.14 (1H, d), 7.46 (1H, d), 6.58 (11H, dd), 6.45 (1H, d), 4.57-4.39 (3H, m), 2.88-2.76 (1H, m), 2.68-2.57 (1H, m), 1.57-1.51 (9H, m).

c) 1,1-Dimethylethyl [(1S)-3-[[3-cyano-6-(trifluoromethyl)-2-pyridinyl]oxy]-1-(hydroxymethyl)-3-(5-isoxazolyl)propyl]-1-carbamate

The sub-title compound was prepared by the method of Example 2 step f) using the product of step b).

MS APCI+ve ^{m}/z 443 [M+H]⁺.

d) 2-[[(3S)-3-Amino-4-hydroxy-1-(5-isoxazolyl)butyl]oxy]-6-(trifluoromethyl)-3-pyridinecarbonitrile, (E)-butenedioate

The title compound was prepared by the method of Example 51, step g) using the product of step c) using. M.p. 150-2° C.

MS APCI+ve ^{m}/z 343 [M+H]⁺.

¹H NMR (DMSO) 8.63 (1H, d), 8.57 (1H, d), 7.74 (1H, d), 6.60 (1H, d), 6.55 (1H, t), 6.47 (2H, s), 3.64-3.49 (2H, m), 3.17-3.09 (1H, m), 2.38 (2H, t).

### EXAMPLE 53

### 2-[[3-(3S)-Amino-4-hydroxy-1-(1R)-(2-thienyl)butyl]oxy]-4-chloro-5-fluorobenzonitrile ethanedioate

a) 1,1-Dimethylethyl 4-[(2R)-2-Hydroxy-2-(2-thienyl)ethyl]-2.2-dimethyl-(4S)-3-oxazolidinecarboxylate

The sub-title compound was prepared from 2-bromothiophene (2.71 g), magnesium (485 mg) and 1,1-dimethylethyl 2,2-dimethyl-4-[(4S)-2-oxoethyl)-3-oxazolidinecarboxylate (3 g) in THF (20 ml) by the method of Example 36, part a) to give an oil (1.51 g).

¹H NMR 300 MHz (d₄-MeOH) 7.31 (1H, dd), 7.03-6.95 (2H, m), 5.00-4.95 (1H, m), 4.15-4.04 (1H, m), 3.92-3.86 (1H, m), 3.81-3.69 (1H, m), 2.35-2.18 (1H, m), 2.01-1.90 (1H, m), 1.56-1.44 (15H, m).

b) 2-[[3-(3S)-Amino-4-hydroxy-1-(1R)-(2-thienyl)butyl]oxy]-4-chloro-5-fluorobenzonitrile ethanedioate

The title compound was prepared from the product from step a) (236 mg) and 4-chloro-2,5-difluorobenzonitrile by the method of Example 36, step b) to give a cream powder (38 mg).

MS APCI+ve ^{m}/z 341 [M+H]⁺.

¹H NMR 300 MHz (d₄-MeOH) 7.63 (1H, d), 7.47 (1H, d), 7.38 (1H, d), 7.24 (1H, d), 7.04-7.01 (1H, m), 6.00 (1H, dd), 3.87 (1H, dd), 3.75-3.69 (1H, m), 3.63-3.55 (1H, m), 2.58-2.48 (1H, m), 2.40-2.31 (1H, m).

### EXAMPLE 54

### 2-[[3-(3S)-Amino-4-hydroxy-1(R)-(3-thienyl)butyl]oxy]-4-chloro-5-fluorobenzonitrile ethanedioate

a) 1,1-Dimethylethyl 4-[(2R)-2-hydroxy-2-(3-thienyl)ethyl]-2,2-dimethyl-(4s)-3-oxazolidinecarboxylate

The sub-title compound was prepared from 3-bromothiophene (1.09 g), 1,1-dimethylethyl 2,2-dimethyl-4-[(4S)-2-oxoethyl)-3-oxazolidinecarboxylate (3 g) in THF (20 ml), and magnesium dibromide by the method of Example 35, step a) to give a yellow oil (158 mg).

¹H NMR 300 MHz (d4-MeOH) 7.40-7.37 (1H, m), 7.28 (1H, s), 7.12 (1H, d), 4.84-4.79 (1H, m), 4.13-3.97 (1H, m), 3.91-3.83 (1H, m), 3.77-3.69 (1H, m), 2.31-2.11 (1H, m), 1.97-1.84 (1H, m), 1.56-1.47 (15H, m).

b) 2-[[3(3S)-Amino-4-hydroxy-1(1R)-(3-thienyl)butyl]oxy]-4-chloro-5-fluorobenzonitrile ethanedioate

The title compound was prepared from the alcohol prepared in step a) (158 mg) and 4-chloro-2,5-difluorobenzonitrile by the method of Example 36, step b) to give a cream powder (30 mg) M.p. 111-115° C.

MS APCI+ve ^{m}/z 341 [M+H]⁺.

¹H NMR 300 MHz (d₄-MeOH) 7.62 (1H, d), 7.51-7.48 (2H, m), 7.25 (1H, d), 7.18-7.16 (1H, m), 5.78-5.75 (1H, m), 3.86 (1H, dd), 3.72-3.67 (1H, m), 3.58-3.53 (1H, m), 2.47-2.40 (1H, m), 2.29-2.23 (1H, m).

### EXAMPLE 55

### 2-[[(1R,3S)-3-Amino-4-hydroxy-1-(3-pyridinyl)butyl]thio]-4-(trifluoromethyl)benzonitrile dihydrochloride

a) 1,1-Dimethylethyl (4S)-4-[(2S)-2-hydroxy-2-(3-pyridinyl)ethyl]-2,2-dimethyl-3-oxazolidinecarboxylate and 1,1-Dimethylethyl (4R)-4-[(2S)-2-hydroxy-2-(3-pyridinyl)ethyl]-2,2-dimethyl-3-oxazolidinecarboxylate

The sub-title compounds were prepared by the method of Example 1 step a) using 3-pyridylmagnesium bromide.

Initial elution of the column gave (2S, 4S) sub-title compound as an oil (3.40 g).

MS APCI+ve ^{m}/z 323 ([M+H]⁺).

¹H NMR 400 MHz (CDCl₃) 8.58 (1H, m), 8.49 (1H, d), 7.75 (1H, d), 7.26 (1H, m), 5.19 (1H, m), 4.68 (1H, m), 4.35 (1H, m), 4.03 (1H, m), 3.67 (1H, d), 2.03 (1H, m), 1.80 (1H, m), 1,62 (3H, s), 1.53 (12H, m).

Further elution of the column gave (2R, 4S) sub-title compound as a pale yellow oil (2.30 g).

MS APCI+ve ^{m}/z 323 ([M+H]⁺).

¹H NMR 400 MHz (CDCl₃) 8.59 (1H, m), 8.51 (1H, d), 7.73 (1H, d), 7.26 (1H, m), 4.83 (1H, m), 3.80-4.20 (4H, m), 2.07 (2H, m), 1,65 (3H, s), 1.50 (12H, m).

b) 1,1-Dimethylethyl (4S)-4-[(2R)-2-(benzoylthio)-2-(3-pyridinyl)ethyl]-2,2-dimethyl-3-oxazolidinecarboxylate

The sub-title compound (2.80 g) was prepared by the method of Example 2 step c) using the (2S,4S) product from step a).

MS APCI+ve ^{m}/z 443 (M+H⁺).

¹H NMR 400 MHz (CDCl₃) 8.68 (1H, d), 8.51 (1H, m), 7.91 (2H, m), 7.72 (1H, m), 7.55 (1H, m), 7.42 (2H, m), 7.26 (1H, m), 4.78 (1H, m), 3.90-4.15 (3H, m), 2.58-2.38 (1H, m), 2.13 (1H, m), 1,60-1.40 (15H, m).

c) 1,1-Dimethylethyl (4S)-4-[(2R)-2-[[2-cyano-5-(trifluoromethyl)phenyl]thio]-2-(3-pyridinyl)ethyl]-2,2-dimethyl-3-oxazolidinecarboxylate

The sub-title compound (180 mg) was prepared by the method of Example 3 step a) using the product from step b) and 2-fluoro-4-(trifluoromethyl)benzonitrile.

MS APCI+ve ^{m}/z 508 (M+H⁺).

d) 2-[[(1R,3S)-3-Amino-4-hydroxy-1-(3-pyridinyl)butyl]thio]-4-(trifluoromethyl)benzonitrile Dihydrochloride

The product from step c) (175 mg) was stirred with methanol (5 ml) and 4 M hydrogen chloride in dioxane (5 ml) for 4 h. The reaction mixture was evaporated and the residue recrystallised from ethanol/diethyl ether to give the title compound (120 mg) as a white solid. M.p. 238-40° C.

MS APCI+ve ^{m}/z 368 [M+H]⁺.

¹H NMR 400 MHz (d₆-DMSO) 8.90 (1H, s), 8.70 (1H, d), 8.40 (1H, m), 8.30 (2H, m), 8.05 (2H, m), 7.78 (2H, m), 5.47 (1H, m), 3.50-3.60 (2H, m), 3.03 (1H, m), 2.40 (2H, m), 2.30 (1H, m).

### EXAMPLE 56

### 2-[[(1R,3S)-3-Amino-4-hydroxy-1-(5-pyrimidyl)butyl]thio]-4-chlorobenzonitrile hydrochloride

a) 1,1-Dimethylethyl 4-[(2S)-2-hydroxy-2-(3-pyridinyl)ethyl]-2,2-dimethyl-(4S)-3-oxazolidinecarboxylate and 1,1-Dimethylethyl 4-[(2R)-2-hydroxy-2-(3-pyridinyl)ethyl]-2,2-dimethyl-(4S)-3-oxazolidinecarboxylate

To a stirred solution of 1,1-dimethylethyl (4S)-2,2-dimethyl-4-(2-oxoethyl)-3-oxazolidinecarboxylate (4.55 g) and 5-bromopyrimidine (3.00 g) in dry THF (50 ml) at −78° C. and under nitrogen was added butyllithium (2.5M in hexanes, 7.90 ml) dropwise. The mixture was stirred at −78° C. for 1.5 hours then quenched with saturated ammonium chloride solution and the products extracted into ethyl acetate. The organic extract was dried (MgSO₄) and concentrated to an oil. The crude mixture of diastereomers was purified by chromatography (silica, methanol/dichloromethane as eluent).

Initial elution of the column gave the (2S,4S) sub-title compound as a yellow solid (1.08 g).

MS APCI+ve ^{m}/z 324 ([M+H]⁺).

¹H NMR 400 MHz (CDCl₃) 9.13 (1H, s), 8.76 (2H, s), 5.41 (1H, m), 4.67 (1H, m), 4.38 (1H, m), 4.06 (1H, dd), 3.68 (1H, d), 2.04 (1H, m), 1.79 (1H, m), 1.62 (3H, s), 1.55 (3H, s), 1.53 (9H, s).

Further elution-of the column gave the (2R,4S) sub-title compound as a pale yellow oil (540 mg).

MS APCI+ve ^{m}/z 324 ([M+H]⁺).

¹H NMR 400 MHz (CDCl₃) 9.13 (1H, s), 8.77 (2H, s), 4.87 (1H, m), 4.67 (1H, m), 4.22 (1H, m), 3.85 (1H, m), 2.15 (2H, m), 1.48-1.60 (15H, m).

b) 1,1-Dimethylethyl (4S)-4-[(2R)-2-(benzoylthio)-2-(5-pyrimidinyl)ethyl1-2,2-dimethyl-3-oxazolidinecarboxylate

The sub-title compound was prepared by the method of Example 2 step c) using the (2S,4S) product from step a).

MS APCI+ve ^{m}/z 444 (M+H⁺).

¹H NMR 400 MHz (CDCl₃) 9.11 (1H, s), 8.81 (2H, m), 7.90 (2H, d), 7.58 (1H, m), 7.44 (2H, m), 4.76 (1H, m), 3.96 (2H, m), 2.40-2.65 (1H, m), 2.16 (1H, m), 1.45-1.80 (16H, m).

c) 1,1-Dimethylethyl (4S)-4-[(2R)-2-[(5-chloro-2-cyanophenyl)thio]-2-(5-pyrimidinyl)ethyl]-2,2-dimethyl-3-oxazolidinecarboxylate

The sub-title compound (200 mg) was prepared by the method of Example 3 step a) using the product from step b) and 4-chloro-2-fluorobenzonitrile.

MS APCI+ve ^{m}/z 475/7 (M+H⁺).

d) 2-[[(1R,3S)-3-Amino-4-hydroxy-1-(5-pyrimidyl)butyl]thio]-4-chlorobenzonitrile hydrochloride

The title compound (90 mg) was prepared as a solid (m.p. 120-30° C.) by the method of Example 7 step c) using the product from step c).

MS APCI+ve ^{m}/z 335/7 [M+H]⁺.

¹H NMR 400 MHz (d₆-DMSO) 9.08 (1H, s), 8.85 (2H, s), 8.23 (3H, bs), 7.90 (1H, d), 7.84 (1H, d), 7.56 (1H, dd), 5.24 (1H, m), 3.50-3.75 (2H, m), 3.01 (1H, m), 2.43 (1H, m), 2,28 (1H, m).

### EXAMPLE 57

### 2-[[(1R,3S)-3-Amino-4-hydroxy-1-(3-pyridinyl)butyl]thio]-4-chloro-5-fluorobenzonitrile dihydrochloride

a) O-(5-Chloro-2-cyano-4-fluorophenyl)dimethylcarbamothioate

A solution of phenol (2.00 g), potassium carbonate (1.85 g) and N,N-dimethylthiocarbamate in acetone (30 ml) was heated to reflux for 24 hours. The mixture was poured into water and extracted with ethyl acetate. The combined organic layers were washed with brine, dried (MgSO₄) and evaporated. The residue was purified by chromatography (silica, isohexane/diethyl ether as eluent) to give the sub-title compound as a solid (2.36 g).

MS APCI+ve ^{m}/z 259/261 [M+H]⁺.

¹H NMR 400 MHz (CDCl₃) 7.43 (1H, d), 7.36 (1H, d), 3.46 (3H, s), 3.40 (3H, s).

b) S-(5-Chloro-2-cyano-4-fluorophenyl)dimethylcarbamothioate

The product from step a) (2.35 g) was heated under reflux under nitrogen in dimethylaniline (25 ml) for 4 hours. The mixture was then poured into 2M HCl solution and extracted with ethyl acetate 3 times. The combined organic layers were washed with brine, dried (MgSO₄) and evaporated to leave the sub-title compound as a white solid (2.3 g).

¹H NMR 400 MHz (CDCl₃) 7.73 (1H, d), 7.52 (1H, d), 3.13 (3H, s), 3.06 (3H, s).

c) 4-Chloro-5-fluoro-2-mercaptobenzonitrile

The product from step b) (2.00 g) was dissolved in methanol (100 ml) and a solution of sodium hydroxide (1.55 g) in water (50 ml) added. The mixture was heated to reflux under nitrogen for 1.5 hours. After cooling the mixture was evaporated and the residue diluted with water and then washed twice with diethyl ether. The aqueous layer was acidified with 2M HCl solution and extracted with ethyl acetate twice. The combined organice extracts were washed with brine, dried (MgSO4) and evaporated to give the sub-title compound (1.45 g).

¹H NMR 400 MHz (CDCl₃) 7.50 (1H, d), 7.40 (1H, d), 4.08 (1H, s).

d) 1,1-Dimethylethyl (4S)-4-[(2R)-2-[(5-chloro-2-cyano-4-fluorophenyl)thio]-2-(3-pyridinyl)ethyl]-2,2-dimethyl-3-oxazolidinecarboxylate

The product from step c) (100 mg) was dissolved in THF (10 ml) and the (2S,4S) product from Example 55 step a) (170 mg) added followed by triphenylphosphine (140 mg) and diethyl azodicarboxylate (0.10 ml). The mixture was stirred at 20° C. for 24 hours and then evaporated. The residue was purified by chromatography (silica, diethyl ether as eluent) to give the sub-title compound as an oil (85 mg).

MS APCI+ve ^{m}/z 492/494 [M+H]⁺.

e) 2-[[(1R,3S)-3-Amino-4-hydroxy-1-(3-pyridinyl)butyl]thio]-4-chloro-5-fluorobenzonitrile dihydrochloride

The title compound (60 mg) was prepared as an off-white solid by the method of Example 55 step d) using the product from step d). M.p. 252-5° C.

MS APCI+ve ^{m}/z 352/4 [M+H]⁺.

¹H NMR 400 MHz (d₆-DMSO) 8.78 (1H, s), 8.67 (1H, d), 8.20 (1H, d), 8.08 (2H, m), 7.72 (1H, dd), 5.21 (1H, t), 3.61-3.37 (2H, m), 3.03 (1H, m), 2.40 (1H, m), 2,25 (1H, m).

### EXAMPLE 58

### 2-[[(1R,3S)-3-Amino-4-hydroxy-1-(3-pyridyl)butyl]thio]-4-bromobenzonitrile dihydrochloride

a) 1,1-Dimethylethyl (4S)-4-[(2R)-2-[(S-bromo-2-cyanophenyl)thio]-2-(3-pyridinyl)ethyl]-2,2-dimethyl-3-oxazolidinecarboxylate

The sub-title compound (170 mg) was prepared by the method of Example 3 step a) using the product from Example 55 step b) and 4-bromo-2-fluorobenzonitrile.

MS APCI+ve ^{m}/z 520/2 (M+H⁺).

¹H NMR 400 MHz (CDCl₃) 8.50-8.30 (1H, m), 7.75-7.57 (5H, m), 7.26 (1H, m), 4.50-3.60 (4H, m), 2.60-2.30 (1H, m), 2.18 (1H, m), 1,60-1.40 (15H, m).

b) 2-[[(1R,3S)-3-Amino-4-hydroxy-1-(3-pyridyl)butyl]thio]-4-bromobenzonitrile dihydrochloride

The title compound (118 mg) was prepared as a white solid by the method of Example 55 step d) using the product from step a). M.p. 278-280° C.

MS APCI+ve ^{m}/z 380 [M+H]⁺.

¹H NMR 400 MHz (d₆-DMSO) 8.94 (1H, s), 8.73 (1H, d), 8.42 (1H, d), 8.32 (3H, bs), 8.03 (1H, s), 7.84 (1H, dd), 7.74 (1H, d), 7.68 (1H, dd), 5.41 (1H, m), 3.60-3.48 (2H, m), 3.02 (1H, m), 2.43 (1H, m), 2,27 (1H, m).

### EXAMPLE 59

### 2-[[(1R,3S)-3-Amino-4-hydroxy-1-(2-thiazolyl)butyl]oxy]-5-fluoro-6-methyl-3-pyridinecarbonitrile hydrochloride

a) Bis(1,1-dimethylethyl) 2-(6-chloro-5-cyano-3-fluoro-2-pyridinyl)propanedioate

To a solution of bis(1,1-dimethylethyl) malonate (1.08 g) in dry DMF (20 ml) was added sodium hydride (200 mg) under nitrogen. The mixture was stirred at 20° C. for 30 minutes then 2,6-dichloro-3-cyano-5-fluoropyridine added. The mixture was stirred for 30 minutes then poured into glacial acetic acid (100 ml) and extracted into ether. The ether layer was dried (MgSO₄) and evaporated. The residue was purified by chromatography (silica, dichloromethane/isohexane as eluent) to give the sub-title compound as a solid (1.38 g).

MS APCI+ve ^{m}/z 369/371 (M+H⁺)

¹H NMR 400 MHz (CDCl₃) 7.72 (1H, d), 4.83 (1H, d), 1.50 (18H, s).

b) 2-Chloro-5-fluoro-6-methyl-3-pyridinecarbonitrile

To the product from step a) (1.3 g) was added trifluoroacetic acid (2 ml) and diphenyl ether (10 g). The mixture was heated under reflux for 10 min. The mixture was dissolved in isohexane, filtered through silica. And the silica was washed with 10% dichloromethane/isohexane followed by dichloromethane. The dichloromethane layer was evaporated to leave a solid which was triturated with cold isohexane to give the sub-title compound (510 mg).

MS APCI+ve ^{m}/z 169/71 (M+H⁺)

¹H NMR 400 MHz (CDCl₃) 7.64 (1H, d), 2.59 (3H, s).

c) 1,1-Dimethylethyl (4S)-4-[(2R)-2-[(3-chloro-5-fluoro-6-methyl-2-pyridinyl)oxy]-2-(2-thiazolyl)ethyl]-2,2-dimethyl-3-oxazolidinecarboxylate

The sub-title compound (180 mg) was prepared by the method of Example 8 step b) using the product from step b) and the (2R,4S) product from Example 8 step a).

MS APCI+ve ^{m}/z 463/5 (M+H⁺).

d) 2-[[(1R,3)-3-Amino-4-hydroxy-1-(2-thiazolyl)butyl]oxy]-5-fluoro-6-methyl -3-pyridinecarbonitrile hydrochloride

The title compound (100 mg) was prepared as a white solid by the method of Example 55 step d) using the product from step c). M.p. 148-50° C.

MS APCI+ve ^{m}/z 323 (M+H⁺).

¹H NMR 400 MHz (d₆-DMSO) 8.38 (1H, d), 8.12 (4H, bs), 7.85 (1H, d), 7.78 (1H, d), 6.60 (1H, m), 3.70 (1H, m), 3.59 (1H, m), 3.35 (1H, m), 2.52-2.43 (5H, m).

### EXAMPLE 60

### 4-[[(1R,3S)-3-Amino-1-(3-fluoro-2-thienyl)-4-hydroxybutyl]thio]-6-methoxy-3-pyridinecarbonitrile (E)-butenedioate salt

a) 3-Fluoro-2-thiothenecarboxylic acid.

The sub-title compound was prepared by the method of reference (OPPI BRIEFS, 1997, 29, 221-223) to yield the sub-title compound (1.5 g, 40%) as a yellow solid. M.p. 171-172° C. (lit. 172-173° C.).

¹H NMR 300 MHz (CDCl₃) δ 7.52 (1H, dd) and 6.89 (1H, d).

b) 3-Fluorothioiphene.

The sub-title compound was prepared by the method of reference (Synth. Comm, 1994, 24, 95-101) to yield the sub-title compound (540 mg, 62%) as a clear liquid.

¹H NMR 300 MHz (CDCl₃) 7.20-7.16 (1H, m), 6.85-6.83 (1H, m) and 6.71-6.69 (1H, m).

c) (4S)-1,1-Dimethethyl 4-[(2S)-2-(3-fluoro-2-thienyl)-2-hydroxyethyl]-2,2-dimethyl-3-oxazolidinecarboxylate.

The sub-title compound was prepared by the method of Example 1 step a) using (4S)-1,1-dimethylethyl-2,2-dimethyl-4-(2-oxoethyl)-3-oxazolidinecarboxyate and 3-fluoro-2-thienyllithium instead of phenyllithium. Purification by chromatography (silica, 10% ethyl acetate/isohexane as eluent) afforded the sub-title compound (500 mg, 28%) as a pale yellow gum.

MS (APCI+ve) m/z 246 [M(+H)]⁺.

¹H NMR 300 MHz (CDCl₃) 7.07 (1H, dd), 6.73 (1H, d), 5.23 (1H, d), 5.03-4.93 (1H, m), 4.38-4.28 (1H, m), 4.04-3.99 (1H, m), 3.70-3.66 (1H, m), 2,20-2.10 (1H, m), 1.96-1.86 (1H, m) and 1.55-1.52 (15H, m).

d) (4S)-1,1-Dimethylethyl 4-[(2R)-2-(acetylthio)-2-(3-fluoro-2-thienyl)-2-ethyl]-2,2-dimethyl-3-oxazolidinecarboxylate.

The sub-title compound was prepared by the method of Example 10 step g) using thioacetic acid and the product of step c) instead of thiobenzoic acid and (4S)-1,1-dimethylethyl 4-[(2S)-2-hydroxy-2-phenylethyl]-2,2-dimethyl-3-oxazolidinecarboxylate. Purification by chromatography (silica, 5% ethyl acetate/isohexane as eluent) afforded the sub-title compound (300 mg) as a colourless oil.

MS (APCI+ve) m/z 304 [M(+H)(-Boc)]⁺.

¹H NMR 300 MHz (CDCl₃) 7.07-7.05 (1H, m), 6.74-6.70 (1H, m), 4.94-4.80 (1H, m), 4.05-3.80 (3H, m), 2.36-2.30 (4H, m), 2.18-2.08 (1H, m) and 1.57-1.47 (15H, m).

e) (4S)-1,1-Dimethylethyl 4-[(2S)-2-[(5-cyano-2-methoxy-4-pyridinyl)thio]-2-(3-fluoro-2-thienyl)ethyl]-2,2-dimethyl-3-oxazolidinecarboxyate.

The sub-title compound was prepared by the method of Example 10 step m) using 6-methoxy-4-(methylsulfonyl)-3-pyridinecarbonitrile and (4S)-1,1-dimethylethyl 4-[(2S)-2-(acetylthio)-2-(3-fluoro-2-thienyl)ethyl]-2,2-dimethyl-3-oxazolidinecarboxyate instead of (4S)-1,1-dimethylethyl 4-[(2S)-2-(benzoylthio)-2-phenylethyl]-2,2-dimethyl-3-oxazolidinecarboxylate. Purification by chromatography (silica, 10% ethyl acetate/isohexane) afforded the sub-title compound (100 mg) as a clear gum.

MS (APCI+ve) m/z 394 [M(+H)(-Boc)]⁺.

¹H NMR 300 MHz (CDCl₃) 8.32-8.30 (1H, m), 7.14-7.10 (1H, m), 6.74-6.70 (2H, m) 5.06-4.64 (1H, m), 4.18-4.08 (1H, m), 4.00-3.85 (4H, m), 3.78-3.48 (1H, m), 2.56-2.15 (2H, m) and 1.58-1.46 (15H, m).

f) 4-[[(1R,3S)-3-Amino-1-(3-fluoro-2-thienyl)-4-hydroxybutyl]thio]-6-methoxy-3-pyridinecarbonitrile (E)-butenedioate salt

The title compound was made by the method of Example 10 step n) to yield the title compound (56 mg) as a white solid. M.p. 177-178° C.

MS (APCI+ve) m/z 354 [M(+H)]⁺.

¹H NMR 300 MHz (d6-DMSO) 8.59 (1H, s), 7.55-7.52 (1H, m), 7.02-6.94 (2H, m), 6.47 (2H, s), 5.45-5.39 (1H, m), 3.92 (3H, s), 3.55-3.35 (1H, m), 3.00-2.90 (1H, m), 2.70-2.60 (1H, m), 2,20-2.10 (1H, m) and 2.05-1.95 (1H, m).

### EXAMPLE 61

### 2-[[(1R,3S)-3-Amino-1-(4-chloro-5-thiazolyl)-4-hydroxybutyl]oxy]-4-chloro-5-fluorobenzonitrile (E)-butenedioate salt

a) 2,4-Dichlorothiazole.

The sub-title compound was prepared by the method of reference (Bull. Chim. Soc. Fr., 1962, 1735) to yield the sub-title compound (7.16 g) as a white solid. M.p. 41-42° C. (lit. 42-43° C.).

¹H NMR 300 MHz (CDCl₃) 7.01 (1H, s).

b) (4S)-1,1-Dimethylethyl 4-[(2R)-2-(2,4-dichloro-5-thiazolyl)-2-hydroxyethyl]-2,2-dimethyl-3-oxazolidinecarboxylate.

The sub-title compound was prepared by the method of Example 1 step a) using (4S)-1,1-dimethylethyl ester-2,2-dimethyl-4-(2-oxoethyl)-3-oxazolidinecarboxylic acid and 2,4-dichloro-5-thiazolyllithium instead of phenyllithium. Purification by chromatography (silica, 20% ethyl acetate in isohexane as eluent) afforded the sub-title compound (744 mg) as a pale yellow gum.

MS (APCI+ve) m/z 297/299/301 [M(+H)(-Boc)]⁺.

¹H NMR 300 MHz (CDCl₃) 5.08-4.98 (1H, m), 4.16-4.04 (2H, m), 3.84-3.71 (1H, m), 2.32-2,22 (2H, m) and 1,61-1.45 (15H, m).

c) (4S)-1,1-Dimethylethyl 4-[(2R)-2-(4-chloro-5-thiazolyl)-2-hydroxyethyl]-2,2-dimethyl-3-oxazolidinecarboxylate.

To a stirred suspension of palladium on activated charcoal (75 mg) and sodium acetate trihydrate (380 mg,) in MeOH (10 ml) was added a solution of the product from step b) (740 mg) in MeOH (15 ml). The mixture was subjected to an atmosphere of hydrogen (5 bar) for 72 h. The mixture was filtered and evaporated to dryness. The residue was dissolved in dichloromethane (25 ml), dried (Na₂SO₄), filtered and concentrated in vacuo. Purification by chromatography (silica, 20% ethyl acetate/isohexane as eluent) afforded the sub-title compound (653 mg) as a colourless gum.

MS (APCI+ve) m/z 363/365 [M(+H)]⁺.

¹H NMR 300 MHz (CDCl₃) 8.63 (1H, s), 5.20-5.10 (1H, m), 4.18-4.04 (2H, m), 3.91-3.84 (1H, m), 2,27-2,20 (2H, m) and 1,62-1.44 (15H, m).

d) (4S)-1,1-Dimethylethyl 4-[(2R)-2-(5-chloro-2-cyano-4-fluorophenoxy)-2-(4-chloro-5-thiazolyl)ethyl]-2,2-dimethyl-3-oxazolidinecarboxylate.

The sub-title compound was prepared by the method of Example 8 step b) using 4-chloro-2,5-difluorobenzonitrile and the product from step b) (650 mg). Purification by chromatography (silica, 20% ethyl acetate/isohexane) afforded the sub-title compound (190 mg) as a pale green foam.

MS (APCI+ve) m/z 416/418/420 [M(+H)(-Boc)]⁺.

¹H NMR 300 MHz (CDCl₃) 9.10 (1H, s), 7.87 (1H, d), 7.39 (1H, d) 5.98(1H, dd), 4.19-4.13 (1H, m), 3.99-3.97 (2H, m), 2.58-2.48 (1H, m), 2.20-2.13 (1H, m) and 1.42-1.40 (15H, m).

e) 2-[[(1R,3S)-3-Amino-1-(4-chloro-5-thiazolyl)-4-hydroxybutyl]thio]-4-chloro-5-fluorobenzonitrile (E)-butenedioate salt

The title compound was made by the method of Example 10 step n) to yield the title compound (136 mg) as a pale yellow solid. M.p. 177-178° C.

MS (APCI+ve) m/z 376/378/380 (M(+H)]⁺.

¹H NMR 300 MHz (d6-DMSO) 9.19 (1H, s), 8.03 (1H, d), 7.65 (1H, d), 6.48 (2H, s), 6.17 (1H, t), 3.60-3.48 (2H, m), 3.10-3.06 (1H, m) and 2.37-2.18 (2H, m).

### EXAMPLE 62

### 2-[[(1R,3S)-3-Amino-4-hydroxy-1-phenylbutyl]thio]-5-nitrobenzonitrile hydrochloride

The title compound was prepared by the method of Example 10 step m) & Example 26 step g) using 2-chloro-4-nitro-benzonitrile and the product from Example 10 step g).

MS (APCI+ve) ^{m}/z 344 [M(+H)]⁺.

¹H 400 MHz (DMSO-d₆) 8.68 (1H, s), 8.38 (1H, d of d), 8.19 (3H, bs), 7.95 (1H, d), 7.58 (2H, d), 7.39 (2H, m), 7.31 (1H, t), 5.35 (2H, m), 3.2-3.52 (2H m), 2.96 (1H, bs), 2.33 (1H, m), 2,22 (1H, m).

### EXAMPLE 63

### 2-[[(1R,3S)-3-Amino-4-hydroxy-1-phenylbutyl]thio]-5-chloro-3-pyridinecarbonitrile-(E)-butenedioate salt

a) 2.5-Dichloro-3-pyridinecarbonitrile

To n-BuLi (1.9 ml of a 2.5M solution in hexanes) in Et₂O (4 ml), under nitrogen, at −78° C., was added a solution of 3-bromo-2,5-dichloro-pyridine (1.08 g) in Et₂O (4 ml) dropwise and stirred for 1.5 h. Solid 1-cyanoimidazole (0.53 g) was added and the reaction stirred for 2 h. After warming to room temperature, water was added and the mixture was extracted with Et₂O. The combined organics were washed with brine, dried (Na₂SO₄) and evaporated to give a black solid (0.64 g). Purification by chromatography (silica, isohexane/Et₂O as eluent) gave the sub-title compound (0.13 g) as a white solid.

¹H NMR 300 MHz (CDCl₃) 8.56 (1H, d), 7.98 (1H, d).

b) 2-[[(1R,3S)-3-Amino-4-hydroxy-1-phenylbutyl]thio]-5-chloro-3-pyridinecarbonitrile-(E)-butenedioate salt

The title compound was prepared by the method of Example 10 steps m & n) using the products from step a) and Example 10 step g).

MS (APCI+ve) ^{m}/z 334 [M(+H)]⁺.

¹H 400 MHz (DMSO-d₆) 8.80 (1H, d), 8.50 (1H, d), 7.50 (2H, d), 7.36 (2H, t), 7.29 (1H, tt), 6.47 (2H, s), 5.32 (1H, dd), 3.44 (1H, dd), 3.35 (1H, dd), 2.79 (1H, m), 2,29 (1H, m), 2.17 (1H, m).

### EXAMPLE 64

### β-Amino-δ-[(4-amino-2-nitrophenyl)thio]-(β¹S,δ¹R)-benzenebutanol

a) 1,1-Dimethylethyl (4S) 4-[(2R)-2-[(4-amino-2-nitrophenyl)thio]-2-phenylethyl]-2,2-dimethyl-3-oxazolidinecarboxylate

The sub-title compound was prepared by the method of Example 10 steps m) using the product from Example 10 step g) and 4-chloro-3-nitroaniline.

MS APCI+ve ^{m}/z 374 ([M+H-boc]⁺).

b) β-Amino-δ-[(4-amino-2-nitrophenyl)thio]-(β¹S,δ¹R)-benzenebutanol

The title compound was prepared by the method of Example 10 step n) using the products from step a).

MS APCI+ve ^{m}/z 334 ([M+H]⁺).

¹H 400 MHz (DMSO-d₆/D₂O) 7.35-7.18 (6H, m), 6.98 (1H, d), 6.72 (1H, dd), 4.54 (1H, t), 3.62-3.36 (2H, m), 2.96 (1H, t), 2.18-2.05 (2H, m).

### EXAMPLE 65

### 2-[[(1R,3S)-3-Amino-4-hydroxy-1-phenylbutyl]thio]-5-bromo-benzonitrile ethanedioate

a) 1,1-Dimethylethyl (4S)-4-[(2R)-2-[(4-bromo-2-cyanophenyl)thio]-2-phenylethyl]-2,2-dimethyl-3-oxazolidinecarboxylate

The product of Example 1 step b) (441 mg) was stirred in 7M NH₃ in methanol (10 ml) at room temperature under nitrogen for 6 h. The mixture was then concentrated in vacuo, the residue dissolved in DMF (10 ml) and treated with 5-bromo-2-fluorobenzonitrile (200 mg), followed by caesium carbonate (650 mg) under nitrogen. The mixture was stirred at room temperature for 20 h and then partitioned between ethyl acetate and water. The separated aqueous phase was extracted with ethyl acetate, and the combined organic extracts were washed with brine, and dried (MgSO₄). The solvent was evaporated and the residue purified by chromatography (silica, 10% ethyl acetate/isohexane) to give the sub-title compound (332 mg, 64%) as a colourless foam oil.

MS APCI+ve m/z 418 [M-BOC+2H]⁺.

b) 1.1-Dimethylethyl [(1S,3R)-3-[(4-bromo-2-cyanophenyl)thio]-1-(hydroxymethyl)-3-phenylpropyl]carbamate

para-Toluenesulfonic acid monohydrate (1 mg) was added to a stirred solution of the product from step a) in methanol (5 ml) under nitrogen, and the mixture was stirred at 20° C. for 48 h. The mixture was diluted with ethyl acetate and washed with 1 M aqueous potassium hydrogensulfate solution, saturated aqueous sodium bicarbonate, brine and then dried (MgSO₄) and evaporated. The resulting residue was purified by chromatography (silica, 30% ethyl acetate/isohexane) to give the sub-title compound (175 mg, 57%) as a colourless foam oil.

MS APCI+ve m/z 378 [M-BOC+2H]⁺.

c) 2-[[(1R,3S)-3-Amino-4-hydroxy-1-phenylbutyl]thio]-5-bromo-benzonitrile ethanedioate

The product from step b) was deprotected according to the procedure of Example 4 step b) to give the title compound (113 mg, 65%) as a white solid.

MS APCI+ve m/z 378 [M+H]⁺.

¹H NMR 300 MHz (D₆-DMSO) 8.11 (1H, d), 7.83 (1H, dd), 7.50 (1H, d), 7.40-7.25 (5H, m), 4.83 (1H, dd), 3.52 (1H, dd), 3.41 (1H, dd), 3.03-2.95 (1H, m), 2.31-2,21 (1H, m), 2.15-2.05 (1H, m).

### Screens

The pharmacological activity of compounds according to the invention was tested in the following screens.

Screen 1

The activity of compounds of formula (I), or a pharmaceutically acceptable salt, enantiomer or racemate thereof, may be screened for nitric oxide synthase inhibiting activity by a procedure based on that of Förstermann et al., Eur. J. Pharm., 1992, 225, 161-165. Nitric oxide synthase converts ³H-L-arginine into ³H-L-citrulline which can be separated by cation exchange chromatography and quantified by liquid scintillation counting.

Enzyme is prepared, after induction, from the cultured murine macrophage cell line J774A-1 (obtained from the laboratories of the Imperial Cancer Research Fund). J774A-1 cells are cultured in Dulbeccos Modified Eagles Medium (DMEM) supplemented with 10% foetal bovine serum, 4 mM L-glutamine and antibiotics (100 units/ml penicillin G, 100 mg/ml streptomycin & 0.25 mg/ml amphotericin B). Cells are routinely grown in 225 cm³ flasks containing 35 ml medium kept at 37° C. and in a humidified atmosphere containing 5% CO₂.

Nitric oxide synthase is produced by cells in response to interferon-g (IFNg) and lipopolysaccharide (LPS). The medium from confluent culture flasks is removed and replaced with 25 ml (per flask) of fresh medium containing 1 mg/ml LPS and 10 units/ml IFNg. After a period of 17-20 hours in culture, harvesting of cells is accomplished by scraping the cell sheet from the flask surface into the culture medium. Cells are collected by centrifugation (1000 g for 10 minutes) and lysate prepared by adding to the cell pellet a solution containing 50 mM Tris-HCl (pH 7.5 at 20° C.), 10% (v/v) glycerol, 0.1% (v/v) Triton-X-100, 0.1 mM dithiothreitol and a cocktail of protease inhibitors comprising leupeptin (2 mg/ml), soya bean trypsin inhibitor (10 mg/ml), aprotinin (5 mg/ml) and phenylmethylsulphonyl fluoride (50 mg/ml).

For the assay, 25 μl of substrate cocktail (50 mM Tris-HCl (pH 7.5 at 20° C.), 400 μM NADPH, 20 μM flavin adenine dinucleotide, 20 μM flavin mononucleotide, 4 μM tetrahydrobiopterin, 12 μM L-arginine and 0.025 mCi L-[³H] arginine) is added to wells of a 96 well filter plate (0.45 μM pore size) containing 25 μl of a solution of test compound in 50 mM Tris-HCl. The reaction is started by adding 50 μl of cell lysate (prepared as above) and after incubation for 1 hour at room temperature is terminated by addition of 50 μl of an aqueous solution of 3 mM nitroarginine and 21 mM EDTA.

Labelled L-citrulline is separated from labelled L-arginine using Dowex AG-SOW. 150 μl of a 25% aqueous slurry of Dowex SOW (Na⁺ form) is added to the assay after which the whole is filtered into 96 well plates. 75 μl of filtrate is sampled and added to wells of 96 well plates containing solid scintillant. After allowing the samples to dry the L-citrulline is quantified by scintillation counting.

In a typical experiment basal activity is 300 dpm per 75 μl sample which is increased to 1900 dpm in the reagent controls. Compound activity is expressed as IC₅₀ (the concentration of drug substance which gives 50% enzyme inhibition in the assay) and aminoguanidie, which gives an IC₅₀ (50% inhibitory concentration) of 10 μM, is tested as a standard to verify the procedure. Compounds are tested at a range of concentrations and from the inhibitions obtained IC₅₀ values are calculated. Compounds that inhibit the enzyme by at least 25% at 100 μM are classed as being active and are subjected to at least one retest.

In the above screen, the compounds of Examples 1 to 10 were tested and gave IC₅₀ values of less than 10 μM indicating that they are expected to show useful therapeutic activity.

Screen 2

Recombinant human NO synthases (iNOS, eNOS & nNOS) were expressed in E. coli and lysates were prepared in Hepes buffer (pH 7.4) containing co-factors (FAD, FMN, H₄B), protease inhibitors, lysozyme and the detergent, CHAPS, These preparations were used, at suitable dilution, to assess inhibition of the various isoforms. Inhibition of NOS was determined by measuring the formation of L-[³H]citrulline from L-[³H]arginine using an adaptation of the method of Förstermann et al.⁹ Enzyme assays were performed in the presence of 3 μM [³H]arginine, 1 mM NADPH and other co-factors required to support NOS activity (FAD, FMN, H₄B, calmodulin, Ca²⁺). Since various NOS inhibitors have been reported to exhibit slow binding kinetics, or to inactivate the enzyme in a time dependent manner, enzyme and inhibitor were pre-incubated for 60 min in the presence of NADPH before addition of arginine to initiate the reaction. Incubations continued for a further 60 min before the assays were quenched and [³H]citrulline separated from unreacted substrate by chromatography on Dowex-SOW resin in a 96-well format.

In the above screen, the compounds of Examples 1 to 65 were tested and gave IC₅₀ values of less than 10 μM against the iNOS enzyme indicating that they are expected to show useful therapeutic activity.

Screen 3

Compounds also show activity against the human form of induced nitric oxide synthase as can be demonstrated in the following assay.

The human colorectal carcinoma cell line, DLD-1 (obtained from the European Collection of Animal Cell Culture—cell line number 90102540) was routinely grown in RPMI 1640 supplemented with 10% (v/v) foetal bovine serum, and 2 mM L-glutamine, at 37° C. in 5% CO₂.

Nitric oxide synthase was induced in cells by addition of medium containing human recombinant gamma-IFN (1000 units/ml), TNF-alpha (200 U/ml), IL-6 (200 U/ml) and IL-1-beta (250 U/ml). After incubation for 18 hours at 37° C., the medium was removed and the cells washed with warm phosphate buffered saline. Cells were incubated for a further 5 hours at 37° C./5% CO₂ in RPMI 1640 containing 100 μM L-arginine and 100 μM verapamil-HCl in the presence and absence of test compounds.

Nitrite accumulation was determined by mixing an equal volume of culture media with Griess reagent (10 mg/ml sulphanilamide, 1 mg N-(1-naphthyl)ethylenediamine in 1 ml 2.5% (v/v) phosphoric acid). Inhibition in the presence of compounds was calculated relative to the nitrite levels produced by untreated cells. IC₅₀ values were estimated from a semi-log plot of % inhibition versus concentration of compound.

In this screen the compounds of Examples 1 to 65 gave IC₅₀ values of less than 100 μM, indicating that they are predicted to show useful therapeutic activity.

1. A compound of formula (I) 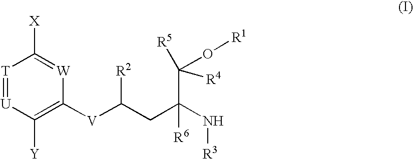
wherein:
X represents H, C1 to 4 alkyl, C1 to 4 alkoxy, halogen, CN, C≡CH, NH₂, NHCH₃, N(CH₃)₂, NO₂, CH₂OH, CHO, COCH₃ or NHCHO; said alkyl or alkoxy group being optionally further substituted by one or more fluorine atoms;
Y represents C1 to 4 alkyl, C1 to 4 alkoxy, halogen, CN, C≡CH, NO₂, CH₂OH, CHO, COCH₃ or NHCHO; said alkyl or alkoxy group being optionally further substituted by one or more fluorine atoms;
T, U and W independently represent CR⁷ or N; and each R⁷ group independently represents H, F or CH₃; and when T represents CR⁷, the group R⁷ may additionally represent OH, Cl, Br, CN, CH₂OH, NO₂, NHR¹³, OR¹⁴ or OSO₂CH₃;
V represents 0 or S(O)ₙ;
n represents an integer 0, 1 or 2;
R¹ represents H or Me;
R² represents C1 to 4 alkyl, C2 to 4 alkenyl, C2 to 4 alkynyl, C3 to 6 cycloalkyl or a 4 to 8 membered saturated heterocyclic ring incorporating one heteroatom selected from O, S and N; any of said groups being optionally further substituted by C1 to 4 alkyl, C1 to 4 alkoxy, C1 to 4 alkylthio, C3 to 6 cycloalkyl, halogen or phenyl; said phenyl group being optionally further substituted by one or more substituents selected independently from halogen, C1 to 4 alkyl, C1 to 4 alkoxy, CF₃, OCF₃, CN or NO₂;
or R² represents phenyl or a five or six membered aromatic heterocyclic ring containing 1 to 3 heteroatoms independently selected from O, S and N; said phenyl or aromatic heterocyclic ring being optionally substituted by one or more substituents selected independently from halogen, C1 to 4 alkyl, C1 to 4 alkoxy, OH, CN, NO₂ or NR⁹R¹⁰; said alkyl or alkoxy group being optionally further substituted by one or more fluorine atoms;
R³ represents H, C1 to 4 alkyl or C3 to 6 cycloalkyl; said alkyl group being optionally substituted by C1 to 4 alkoxy, halogen, hydroxy, NR¹¹ R¹², phenyl or a five or six membered aromatic or saturated heterocyclic ring containing 1 to 3 heteroatoms independently selected from O, S and N; said phenyl or aromatic heterocyclic ring being optionally further substituted by halogen, C1 to 4 alkyl, C1 to 4 alkoxy, CF₃, OCF₃, CN or NO₂.
R⁴, R⁵, R⁶, R⁹, R¹⁰, R¹¹, R¹², R¹³ and R¹⁴ independently represent H or C1 to 4 alkyl;
or a pharmaceutically acceptable salt, enantiomer or racemate thereof.

2. A compound of formula (I), according to claim 1, wherein V represents O.

3. A compound of formula (I), according to claim 1, wherein V represents S(O)ₙ and n represents 0.

4. A compound of formula (I), according to claim 1, wherein X and Y independently represent Br, Cl, CH₃, CH₂F, CHF₂, CF₃, OCH₃ or CN.

5. A compound according to claim 4 wherein Y represents CN.

6. A compound of formula (I), according to claim 1, which is:
2-[[(1R,3S)-3-amino-4-hydroxy-1-phenylbutyl]thio]-6-methyl-3-pyridinecarbonitrile;
2-[[(3S)-3-amino-4-hydroxy-1-(3-isoxazolyl)butyl]thio]-6-methyl-3-pyridinecarbonitrile;
4-[[(1R,3S)-3-amino-4-hydroxy-1-phenylbutyl]thio]-6-methyl-3-pyridinecarbonitrile;
3-[[(1R,3S)-3-amino-4-hydroxy-1-phenylbutyl]thio]-5-(trifluoromethyl)-2-pyridinecarbonitrile;
2-[[(1R,3S)-3-amino-4-hydroxy-1-phenylbutyl]thio]-6-(difluoromethyl)-3-pyridinecarbonitrile;
2-[[(1R,3S)-3-amino-4-hydroxy-1-phenylbutyl]thio]-6-(fluoromethyl)-3-pyridinecarbonitrile;
2-[[(1R,3S)-3-amino-4-hydroxy-1-(3-pyridinyl)butyl]oxy]-4-chloro-5-fluorobenzonitrile;
2-[[(1R,3S)-3-amino-4-hydroxy-1-(2-thiazolyl)butyl]oxy]-4-chloro-5-fluorobenzonitrile;
2-[[(1R,3S)-3-amino-4-hydroxy-1-(5-isothiazolyl)butyl]oxy]-4-chloro-5-fluorobenzonitrile;
4-[[(1R,3S)-3-amino-4-hydroxy-1-phenylbutyl]thio]-6-methoxy-3-pyridinecarbonitrile;
4-[[(1R,3R)-3-amino-4-hydroxy-1-phenylbutyl]thio]-6-methoxy-3-pyridinecarbonitrile;
4-[[(1S,3R)-3-amino-4-hydroxy-1-phenylbutyl]thio]-6-methoxy-3-pyridinecarbonitrile;
4-[[(1S,3S)-3-amino-4-hydroxy-1-phenylbutyl]thio]-6-methoxy-3-pyridinecarbonitrile;
4-[[(1R,3S)-3-amino-4-hydroxy-1-phenylbutyl]thio]-6-(difluoromethoxy)-3-pyridinecarbonitrile;
2-[[(1R,3R)-3-amino-4-hydroxy-1-phenylbutyl]thio]-6-methyl-3-pyridinecarbonitrile;
4-[[(1R,3S)-3-amino-4-hydroxy-1-phenylbutyl]thio]-6-(²H₃)methoxy-3-pyridinecarbonitrle;
2-[[(1R,3S)-3-amino-4-hydroxy-1-phenylbutyl]thio]-6-ethyl-3-pyridinecarbonitrile;
2-[[(1R,3S)-3-amino-4-hydroxy-1-phenylbutyl]thio]-6-(1-methylethyl)-3-pyridinecarbonitrile;
2-[[(1R,3S)-3-amino-4-hydroxy-1-phenylbutyl]thio]-6-methyl-3-pyridinemethanol;
6-acetyl-2-[[(1R,3S)-3-amino-4-hydroxy-1-phenylbutyl]thio]-3-pyridinecarbonitrile;
2-[[(1R,3S)-3-amino-4-hydroxy-1-phenylbutyl]thio]-6-(hydroxymethyl)-3-pyridinecarbonitrile;
2-[[(1R,3S)-3-amino-4-hydroxy-1-phenylbutyl]thio]-3-pyridinecarbonitrile;
(β¹S,δ¹R)-β-amino-δ-[(2,5-dichloro-4-pyridinyl)thiobenzenebutanol];
2-[[(1R,3S)-3-amino-4-hydroxy-1-phenylbutyl]thio]-5-fluoro-6-methoxy-3-pyridinecarbonitrile;
4-[[(1R,3S)-3-amino-4-hydroxy-1-phenylbutyl]thio]-6-(dimethylamino)-3-pyridinecarbonitrile;
4-[[(1R,3S)-3-amino-4-hydroxy-1-phenylbutyl]thio]-6-(methylamino)-3-pyridinecarbonitrile;
(β¹S,δ¹R)-β-amino-δ-[(5-bromo-2-methoxy-4-pyridinyl)thio]-benzenebutanol;
(β¹S,δ¹R)-β-amino-δ-[(5-chloro-2-methoxy-4-pyridinyl)thio]-benzenebutanol;
4-[[(1R,3S)-3-amino-4-hydroxy-1-phenylbutyl]thio]-6-ethoxy-3-pyridinecarbonitrile;
3-[[(1R,3S)-3-amino-4-hydroxy-1-phenylbutyl]thio]-5-(trifluoromethyl)-2-pyridinecarbonitrile;
3-[[(1R,3S)-3-amino-4-hydroxy-1-phenylbutyl]thio]-1,6-dihydro-5-methyl-6-oxo-2-pyridinecarbonitrile;
3-[[(1R,3S)-3-amino-4-hydroxy-1-phenylbutyl]thio]-5-chloro-2-pyridinecarbonitrile;
6-amino-4-[[(1R,3S)-3-amino-4-hydroxy-1-phenylbutyl]thio]-3-pyridinecarbonitrile;
3-[[(1R,3S)-3-amino-4-hydroxy-1-phenylbutyl]thio]-5-methyl-2-pyridinecarbonitrile;
4-[[(1R,3S)-3-amino-1-(2-fluorophenyl)-4-hydroxybutyl]thio]-6-methoxy-3-pyridinecarbonitrile;
2-[[(1R,3S)-3-amino-1-(4-fluorophenyl)-4-hydroxybutyl]oxy]-6-trifluoromethyl-3-pyridinecarbonitrile;
2(2S)-amino-4(4R)-(3-fluorophenyl)-4-[(4-methoxy-2-nitrophenyl)thio]butan-1-ol;
2(2S)-amino-4(4R)-(3-fluorophenyl)-4-[(4-chloro-2-nitrophenyl)thio]butan-1-ol;
2(2S)-amino-4(4R)-(3-fluorophenyl)-4-[(5-amino-4-chloro-2-nitrophenyl)thio]butan-1-ol;
2(2S)-amino-4(4R)-(3-fluorophenyl)-4-[(4-hydroxymethyl)-2-nitrophenyl)thio]butan-1-ol;
2(2S)-amino-4(4R)-(3-fluorophenyl)-4-[(4-fluoro-2-nitrophenyl)thio]butan-1-ol;
2(2S)-amino-4(4R)-(3-fluorophenyl)-4-[(3,5-dichloro-2-pyridyl)thio]butan-1-ol;
4-[[(1R,3S)-3-amino-4-hydroxy-1-phenylbutyl]thio]-3-chlorobenzonitrile;
4-chloro-2-[[(1R,3S)-3-(ethylamino)-4-hydroxy-1-(2-thiazolyl)butyl]oxy]-5-fluoro-benzonitrile;
2-[[(1R,3S)-3-amino-4-hydroxy-1-(5-thiazolyl)butyl]oxy]-5-fluoro-benzonitrile;
2-[[(1R,3S)-3-amino-4-methoxy-1-phenylbutyl]thio]-6-methyl-3-pyridinecarbonitrile;
2-[[(1R,3S)-3-amino-4-hydroxy-4-methyl-1-phenylpentyl]oxy]-4-chloro-5-fluorobenzonitrile;
2-[[(1S,3S)-3-amino-4-hydroxy-1-propylbutyl]oxy]-4-chloro-5-fluorobenzonitrile;
2-[[(1S)-1-[(2S)-2-amino-3-hydroxypropyl]pentyl]thio]-6-methyl-3-pyridinecarbonitrile;
2-[[(1S,3S)-3-amino-4-hydroxy-1-(2-methylpropyl)butyl]thio]-6-methyl-3-pyridinecarbonitrile;
2-[[(3S)-3-amino-4-hydroxy-1-(5-isoxazolyl)butyl]thio]-6-methyl-3-pyridinecarbonitrile;
2-[[(3S)-3-amino-4-hydroxy-1-(5-isoxazolyl)butyl]oxy]-6-(trifluoromethyl)-3-pyridinecarbonitrile;
2-[[3-(3S)-amino-4-hydroxy-1-(1R)-(2-thienyl)butyl]oxy]-4-chloro-5-fluorobenzonitrile;
2-[[3-(3S)-amino-4-hydroxy-1(1R)-(3-thienyl)butyl]oxy]-4-chloro-5-fluorobenzonitrile;
2-[[(1R,3S)-3-amino-4-hydroxy-1-(3-pyridinyl)butyl]thio]-4-(trifluoromethyl)benzonitrile;
2-[[(1R,3S)-3-amino-4-hydroxy-1-(5-pyrimidyl)butyl]thio]-4-chlorobenzonitrile;
2-[[(1R,3S)-3-amino-4-hydroxy-1-(3-pyridinyl)butyl]thio]-4-chloro-5-fluorobenzonitrile;
2-[[(1R,3S)-3-amino-4-hydroxy-1-(3-pyridyl)butyl]thio]-4-bromobenzonitrile;
2-[[(1R,3S)-3-amino-4-hydroxy-1-(2-thiazolyl)butyl]oxy]-5-fluoro-6-methyl-3-pyridinecarbonitrile;
4-[[(1R,3S)-3-amino-1-(3-fluoro-2-thienyl)-4-hydroxybutyl]thio]-6-methoxy-3-pyridinecarbonitrile;
2-[[(1R,3S)-3-amino-1-(4-chloro-5-thiazolyl)-4-hydroxybutyl]oxy]-4-chloro-5-fluorobenzonitrile;
2-[[(1R,3S)-3-amino-4-hydroxy-1-phenylbutyl]thio]-5-nitrobenzonitrile;
2-[[(1R,3S)-3-amino-4-hydroxy-1-phenylbutyl]thio]-5-chloro-3-pyridinecarbonitrile;
β-amino-δ-[(4-amino-2-nitrophenyl)thio]-(β¹S,δ¹R)-benzenebutanol;
2-[[(1R,3S)-3-amino-4-hydroxy-1-phenylbutyl]thio]-5-bromo-benzonitrile;
or a pharmaceutically acceptable salt, enantiomer or racemate thereof.

7. (Canceled)

8. A pharmaceutical composition comprising a compound of formula (I) according to claim 1, or a pharmaceutically acceptable salt, enantiomer or racemate thereof, in admixture with a pharmaceutically acceptable adjuvant, diluent or carrier.

9-16. (Canceled).

17. A method of treating, or reducing the risk of human diseases or conditions in which inhibition of nitric oxide synthase activity is beneficial which comprises administering a therapeutically effective amount of a compound of formula (I), as defined in claim 1, or a pharmaceutically acceptable salt, enantiomer or racemate thereof, to a person suffering from, or at increased risk of, such diseases or conditions.

18. A method of treatment according to claim 17 in which it is predominantly inducible nitric oxide synthase that is inhibited.

19. A method of treating, or reducing the risk of, inflammatory disease in a person suffering from, or at risk of, said disease, wherein the method comprises administering to the person a therapeutically effective amount of a compound of formula (I), as defined in claim 1, or a pharmaceutically acceptable salt, enantiomer or racemate thereof.

20. The method of treatment as claimed in claim 19 wherein the disease is inflammatory bowel disease.

21. The method of treatment as claimed in claim 19 wherein the disease is rheumatoid arthritis.

22. The method of treatment as claimed in claim 19 wherein the disease is osteoarthritis.

23. A method of treating, or reducing the risk of, pain in a person suffering from, or at risk of, said condition, wherein the method comprises administering to the person a therapeutically effective amount of a compound of formula (I), as defined in claim 1, or a pharmaceutically acceptable salt, enantiomer or racemate thereof.

24. A method of treating, or reducing the risk of, inflammatory disease in a person suffering from, or at risk of, said disease, wherein the method comprises administering to the person a therapeutically effective amount of a combination of a compound of formula (I), as defined claim 1, or a pharmaceutically acceptable salt, enantiomer or racemate thereof, with a COX-2 inhibitor.

25. A process for the preparation of a compound of formula (I), as defined in claim 1, or a pharmaceutically acceptable salt, enantiomer or racemate thereof, wherein the process comprises:
(a) reaction of a compound of formula (II) 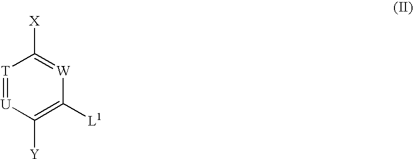
wherein T, U, X, Y and W are as defined in claim 1 and L¹ represents a leaving group, with a compound of formula (III) 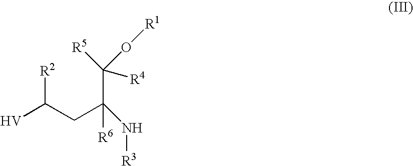
wherein R¹, R², R³, R⁴, R⁵, R⁶ and V are as defined in claim 1; or
(b) reaction of a compound of formula (IV) 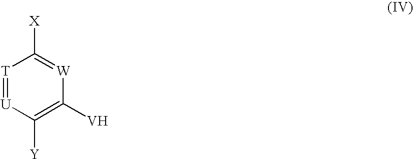
wherein T, U, W, X, Y and V are as defined in claim 1; with a compound of formula (V) 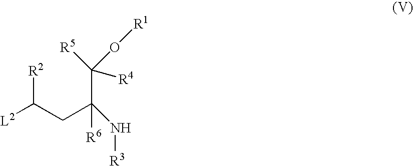
wherein R¹, R², R³, R⁴, R₅ and R⁶ are as defined in claim 1 and L² is a leaving group;
and where desired or necessary converting the resultant compound of formula (I), or another salt thereof, into a pharmaceutically acceptable salt thereof; or converting one compound of formula (I) into another compound of formula (I); and where desired converting the resultant compound of formula (I) into an optical isomer thereof.

26. A compound of formula (I), according to claim 2, wherein X and Y independently represent Br, Cl, CH₃, CH₂F, CHF₂, CF₃, OCH₃ or CN.

27. A compound of formula (I), according to claim 3, wherein X and Y independently represent Br, Cl, CH₃, CH₂F, CHF₂, CF₃, OCH₃ or CN.

28. A compound of formula (I), according to claim 26, wherein Y represents CN.

29. A compound of formula (I), according to claim 27, wherein Y represents CN.
